(19) United States
(12) Patent Application Publication
(10) Pub. No.: US 2025/0263790 A1
(43) Pub. Date: Aug. 21, 2025

(54) METHODS OF DETERMINING SEQUENCE INFORMATION

(71) Applicant: Illumina, Inc., San Diego, CA (US)

(72) Inventors: Jonathan BOUTELL, Cambridge (GB); Niall GORMLEY, Cambridge (GB); Gery VESSERE, San Diego, CA (US); Aathavan KARUNAKARAN, San Diego, CA (US); Eli CARRAMI, Cambridge (GB); Oliver MILLER, Cambridge (GB); Stephen BRUINSMA, San Diego, CA (US); Shagesh SRIDHARAN, San Diego, CA (US); Nileshi SARAF, San Diego, CA (US)

(21) Appl. No.: 18/573,965

(22) PCT Filed: Mar. 15, 2023
(86) PCT No.: PCT/EP2023/056653

§ 371 Date: Dec. 22, 2023

Related U.S. Application Data
(60) Provisional application No. 63/439,417, filed on Jan. 17, 2023.
(60) Provisional application No. 63/439,438, filed on Jan. 17, 2023.
(60) Provisional application No. 63/439,443, filed on Jan. 17, 2023.
(60) Provisional application No. 63/439,466, filed on Jan. 17, 2023.
(60) Provisional application No. 63/439,501, filed on Jan. 17, 2023.
(60) Provisional application No. 63/439,519, filed on Jan. 17, 2023.
(60) Provisional application No. 63/439,415, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/269,383, filed on Mar. 15, 2022.

(51) Int. Cl. C12Q 1/6869; G16B 30/10; G16B 40/10

(52) U.S. Cl. CPC C12Q 1/6869; G16B 30/10; G16B 40/10

(57) ABSTRACT

A method of determining sequence information from two or more polynucleotide sequence portions, the method comprising: selecting one of a plurality of classifications based on first and second intensity data, wherein each classification represents one or more possible combinations of respective nucleobases of the two or more polynucleotide sequence portions, and wherein at least one classification represents more than one possible combination of respective nucleobases.

### CROSS-REFERENCE TO RELATED APPLICATIONS

Any and all priority claims identified in the Application Data Sheet, or any correction thereto, are hereby incorporated by reference under 37 CFR 1.57. This application is the national phase under 35 U.S.C. § 371 of prior PCT International Application No. PCT/EP2023/056653 which has an International Filing Date of Mar. 15, 2023, which designates the United States of America, and which claims priority to U.S. Provisional Application No. 63/269,383 filed Mar. 15, 2022. U.S. Provisional Application No. 63/439,443 filed Jan. 17, 2023, U.S. Provisional Application No. 63/439,417 filed Jan. 17, 2023, U.S. Provisional Application No. 63/439,438 filed Jan. 17, 2023, U.S. Provisional Application No. 63/439,415 filed Jan. 17, 2023, U.S. Provisional Application No. 63/439,466 filed Jan. 17, 2023, U.S. Provisional Application No. 63/439,519 filed Jan. 17, 2023, U.S. Provisional Application No. 63/439,491 filed Jan. 17, 2023, U.S. Provisional Application No. 63/439,522 filed Jan. 17, 2023, and U.S. Provisional Application No. 63/439,501 filed Jan. 17, 2023. Each of the aforementioned applications is incorporated by reference herein in its entirety, and each is hereby expressly made a part of this specification.

### REFERENCE TO SEQUENCE LISTING

The present application is being filed along with a Sequence Listing in electronic format. The Sequence Listing is provided as a file entitled “Sequence Listing final v4 PC932621US.xml”, which was created on Mar. 15, 2023 and is approximately 167 kilobytes in size, and is replaced by a file entitled “Sequence Listing final PC932621USA-corrected.xml”, which was created on Nov. 8, 2024 and is approximately 167 kilobytes in size. The information in the electronic format of the Sequence Listing is hereby incorporated by reference in its entirety.

### BACKGROUND

### Field

The disclosed technology relates to the field of nucleic acid sequencing. More particularly, the disclosed technology relates to using next generation sequencing to determine sequence information from two or more polynucleotide sequence portions in a single sequencing run.

### Description of the Related Art

In some types of next-generation sequencing (NGS) technologies, a nucleic acid cluster is created on a flow cell by amplifying an original template nucleic acid strand. Sequencing cycles may be performed as complementary strands of the template nucleic acids are being synthesized, i.e., using sequencing-by-synthesis (SBS) processes.

In each sequencing cycle, deoxyribonucleic acid analogs conjugated to fluorescent labels are hybridized to the template nucleic acids, and excitation light sources are used to excite the fluorescent labels on the deoxyribonucleic acid analogs. Detectors capture fluorescent emissions from the fluorescent labels and identify the deoxyribonucleic acid analogs. As a result, the sequence of the template nucleic acids may be determined by repeatedly performing such sequencing cycles.

NGS allows for the sequencing of a number of different template nucleic acids simultaneously, significantly reducing the cost of sequencing in the last twenty years, however there remains a desire for further increases in sequencing throughput.

### SUMMARY

According to a first aspect of the present invention, there is provided a method of determining sequence information from two or more polynucleotide sequence portions, the method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the at least one first polynucleotide sequence portion and the at least one second polynucleotide sequence portion.

In embodiments, the first and second signals and/or the third and fourth signals may be obtained substantially simultaneously.

In embodiments, selecting the classification based on the first and second intensity data may comprise selecting the classification based on the combined intensity of the first and second signals and the combined intensity of the third and fourth signals.

In embodiments, when based on a nucleobase of the same identity, an intensity of the first signal may be substantially the same as an intensity of the second signal and an intensity of the third signal may be substantially the same as an intensity of the fourth signal.

In embodiments, the plurality of classifications may consist of a predetermined number of classifications.

In embodiments, the plurality of classifications may comprise:
one or more classifications representing matching first and second nucleobases; and
one or more classifications representing mismatching first and second nucleobases, and
wherein determining sequence information from the at least one first polynucleotide sequence portion and the at least one second polynucleotide sequence portion comprises:
   in response to selecting a classification representing matching first and second nucleobases, determining a match between the first and second nucleobases; or
   in response to selecting a classification representing mismatching first and second nucleobases, determining a mismatch between the first and second nucleobases.

In embodiments, determining sequence information from the at least one first polynucleotide sequence portion and the at least one second polynucleotide sequence portion may comprise, in response to selecting a classification representing a match between the first and second nucleobases, base calling the first and second nucleobases.

In embodiments, determining sequence information from the at least one first polynucleotide sequence portion and the at least one second polynucleotide sequence portion may comprise, based on the selected classification, determining that the second polynucleotide sequence portion is modified relative to the first polynucleotide sequence portion at a location associated with the first and second nucleobases.

Said modification may have been made to any of the first polynucleotide sequence portion, the second polynucleotide sequence portion, or any sequence from which either of the first and second portions are derived, provided that it results in the modification of the sequences of the first and second portions relative to one another.

In embodiments, the second polynucleotide sequence portion may be modified relative to the first polynucleotide sequence portion resulting from a library preparation and/or sequencing error.

In embodiments, the second polynucleotide sequence portion may be modified relative to the first polynucleotide sequence portion resulting from conversion of a modified cytosine to thymine or a nucleobase which is read as thymine/uracil, and/or of an unmodified cytosine to uracil or a nucleobase which is read as thymine/uracil.

In embodiments, at least one polynucleotide sequence may comprise the first polynucleotide sequence portion and the second polynucleotide sequence portion.

In embodiments, the at least one polynucleotide sequence may comprise portions of a double-stranded nucleic acid template, and the first polynucleotide sequence portion may comprise a forward strand of the template, and the second polynucleotide sequence portion may comprise a reverse complement strand of the template; or the first polynucleotide sequence portion may comprise a reverse strand of the template, and the second polynucleotide sequence portion may comprise a forward complement strand of the template,
the template may be generated from a target polynucleotide to be sequenced via complementary base pairing, and the target polynucleotide may have been pre-treated using a conversion reagent, and
the conversion reagent may be configured to convert a modified cytosine to thymine or a nucleobase which is read as thymine/uracil, and/or the conversion reagent may be configured to convert an unmodified cytosine to uracil or a nucleobase which is read as thymine/uracil.

In embodiments, at least one first polynucleotide sequence may comprise the first polynucleotide sequence portion and at least one second polynucleotide sequence may comprise the second polynucleotide sequence portion.

In embodiments, the at least one first polynucleotide sequence and the at least one second polynucleotide sequence may each comprise portions of a double-stranded nucleic acid template, and the first polynucleotide sequence portion may comprise a forward strand of the template, and the second polynucleotide sequence portion may comprise a reverse complement strand of the template; or the first polynucleotide sequence portion may comprise a reverse strand of the template, and the second polynucleotide sequence portion may comprise a forward complement strand of the template,
the template may be generated from a target polynucleotide to be sequenced via complementary base pairing, and the target polynucleotide may have been pre-treated using a conversion reagent, and
the conversion reagent may be configured to convert a modified cytosine to thymine or a nucleobase which is read as thymine/uracil, and/or the conversion reagent may be configured to convert an unmodified cytosine to uracil or a nucleobase which is read as thymine/uracil.

In embodiments, the first signal, second signal, third signal and fourth signal may be generated based on light emissions associated with the respective nucleobase and detected at a sensor.

In embodiments, the obtained signals may be generated by:
contacting a plurality of polynucleotide molecules comprising the first and second polynucleotide sequence portions with first primers for sequencing the first polynucleotide sequence portion and second primers for sequencing the second polynucleotide sequence portion;
extending the first primers and the second primers by contacting the polynucleotide molecules with labeled nucleobases to form first labeled primers and second labeled primers;
stimulating the light emissions from the first and second labeled primers; and
detecting the light emissions at a sensor.

In embodiments,
the first and second signals may be based on light emissions detected in a first range of optical frequencies;
the third and fourth signals may be based on light emissions detected in a second range of optical frequencies; and
the first range of optical frequencies and the second range of optical frequencies may be not identical.

For example, the first range of optical frequencies may correspond to the color red, e.g., 400-484 THz (or equivalently, 620-750 nm in terms of wavelength), and the second range of optical frequencies may correspond to the color green, e.g., 526-806 THz (or equivalently, 495-570 nm in terms of wavelength).

In embodiments, the polynucleotide molecules comprising the first and second polynucleotide sequence portions may be attached to a substrate, optionally a flow cell.

In embodiments, the light emissions from the first labeled primers and the light emissions from the second labeled primers may be emitted from the same region or substantially overlapping regions of the substrate.

In embodiments, the light emissions detected at the sensor may be spatially unresolved.

In embodiments, the sensor may be configured to provide a single output based upon the first and second signals.

In embodiments, the sensor may comprise a single sensing element.

In embodiments, the at least one first polynucleotide sequence portion and the at least one second polynucleotide sequence portion may be present in a cluster.

In embodiments, the one of the plurality of classifications may be selected based on the first and the second intensity data using a Gaussian mixture model.

In embodiments, the method may further comprise repeating steps (a) to (d) for each of a plurality of base calling cycles.

According to a second aspect of the present invention, there is provided a method of detecting sequence modifications, the method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion, wherein, in the absence of a sequence modification, the sequences of the first and second polynucleotide sequence portions are the same;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion; and
(c) based on the first and second intensity data, identifying the presence or absence of a sequence modification.

In embodiments, (c) may comprise:
classifying the combined intensity of the first and second signals and the combined intensity of the third and fourth signals as being of high, intermediate, or low intensity; and
in response to classifying one or both of the combined intensity of the first and second signals and the combined intensity of the third and fourth signals as being of intermediate intensity, determining the presence of a sequence modification.

In embodiments, the method may further comprise repeating steps (a) to (c) for each of a plurality of base calling cycles.

According to a third aspect of the present invention, there is provided a data processing device comprising means for carrying out a method as described above.

In embodiments, the data processing device may be a polynucleotide sequencer.

According to a fourth aspect of the present invention, there is provided a computer program product comprising instructions which, when the program is executed by a processor, cause the processor to carry out a method as described above.

According to a fifth aspect of the present invention, there is provided a computer-readable storage medium comprising instructions which, when executed by a processor, cause the processor to carry out a method as described above.

According to a sixth aspect of the present invention, there is provided a computer-readable data carrier having stored thereon a computer program product as described above.

According to a seventh aspect of the present invention, there is provided a data carrier signal carrying a computer program product as described above.

In one embodiment, the disclosed technology provides systems and methods for determining sequence information from two or more polynucleotide sequence portions in parallel (i.e., substantially simultaneously, using the same sequencing run), while the two sequence portions are co-localized within the same nucleic acid cluster. The sequence information may be one or more nucleobase sequences of the polynucleotide sequence portions and/or may be additional sequence information determined simultaneously with one or more nucleobase sequences of the polynucleotide sequence portions.

In one embodiment, the disclosed method can include providing a substrate comprising a plurality of single or double stranded polynucleotide molecules in a cluster. The disclosed method can further include contacting the plurality of polynucleotide molecules with first primers for sequencing a first polynucleotide sequence portion and second primers for sequencing a second polynucleotide sequence portion. The first and second polynucleotide sequence portions may be present as respective portions of the same polynucleotide molecules. Alternatively, the first and second polynucleotide sequence portions may be present in different polynucleotide molecules of the plurality of polynucleotide molecules. The disclosed method can further include extending the first primers and the second primers by contacting the cluster with labeled nucleobases to form first labeled primers and second labeled primers. The disclosed method can further include stimulating light emissions from the first and second labelled primers. The disclosed method can further include determining sequence information based on the amplitude of the signal generated by the labelled nucleobases. In some embodiments, the first primers are index primers that hybridize to a site adjacent to a barcode index portion associated with the first sequence portion. In some embodiments, the second primers are index primers that hybridize to a site adjacent to a barcode index portion associated with the second sequence portion. In some embodiments, the first primers are index primers that hybridize to a site adjacent to a barcode index portion associated with the first sequence portion, and the second primers are index primers that hybridize to a site adjacent to a barcode index portion associated with the second sequence portion.

The second polynucleotide sequence portion and first polynucleotide sequence portion may be derived from a common sequence. The second polynucleotide sequence portion may be modified with respect to the first polynucleotide sequence portion, for example by a modification introduced in producing the second polynucleotide sequence portion that is not introduced in producing the first polynucleotide sequence portion.

In some embodiments, the sequence information may identify the labeled nucleobases added to the first primers and second primers where the nucleobases are the same. Additional or alternative useful information may be determined, where the nucleobases are the same or where the nucleobases differ. This other useful information provided by the sequence information may take various forms. For example, the sequence information may provide information associated with the presence of differences or similarities between the nucleobases at a given position. In some embodiments, the first and second polynucleotide sequence portions may be related (for example, originating from a common polynucleotide molecule), but may differ at one or more corresponding positions due to the way in which the sequences have been processed prior to sequencing. The sequence information may indicate the locations at which the nucleobases are the same or differ as a result of this processing. Within these two possibilities, even more detailed information may be determined. In particular, by using a priori knowledge of the processing used to obtain the first and second polynucleotide sequence portions (e.g. from an original double-stranded target molecule), in some embodiments it is possible to determine a property associated with that particular location. For example, in some embodiments, an original double-stranded target molecule may have been processed to convert either methylated or unmethylated cytosines to a different base. The first polynucleotide sequence portion may correspond to the sequence of a forward strand (or a reverse strand) of the converted double-stranded molecule, and the one or more second polynucleotide sequence portions may correspond to a reverse complement strand (or a forward complement strand). Based upon the combined amplitude of the signals obtained from each sequence portion, it is possible to determine the methylation status of cytosines in the original target molecule at the corresponding location. In other embodiments, the processing may be processing that is typically undertaken during sample preparation, for example during amplification, and the sequence information may indicate locations where the processing caused a modification. The sequence information may therefore provide both sequence information and identification of sample preparation errors. In other embodiments, the first and second polynucleotides sequence portions may be similar sequences (e.g. obtained from different sources). The sequence information may then provide, as an output, data identifying nucleobases where the sequences are identical in a particular location, and data indicating locations where the first and second polynucleotide sequence portions vary otherwise, thereby providing instantaneous variant identification between two similar sequences.

In some embodiments, the signal generated by the first labelled primers and the signal generated by the second labeled primers are emitted from the same region or substantially overlapping regions of the substrate. In some embodiments, each polynucleotide molecule is attached to the substrate. In some embodiments, the plurality of polynucleotide molecules in the cluster are generated by a bridge amplification process, an exclusion amplification process, a rolling circle amplification process, or any other suitable amplification process. In some embodiments, the substrate comprises a plurality of clusters of nucleic acids, the clusters being randomly distributed on the substrate. In alternative embodiments, the clusters are arranged in a patterned array.

In some embodiments, the disclosed method further includes: detecting the signal generated by the first labeled primers in a first range of optical frequencies and a second range of optical frequencies; and detecting the signal generated by the second labeled primers in the first range of optical frequencies and the second range of optical frequencies, wherein the first range of optical frequencies and the second range of optical frequencies are not identical. For example, the first range of optical frequencies may correspond to the color red, e.g., 400-484 THz (or equivalently, 620-750 nm in terms of wavelength), and the second range of optical frequencies may correspond to the color green, e.g., 526-606 THz (or equivalently, 495-570 nm in terms of wavelength).

In some embodiments, the disclosed method further includes: acquiring a first fluorescent image of the cluster in a first range of optical frequencies; acquiring a second fluorescent image of the cluster in a second range of optical frequencies, wherein the first range of optical frequencies and the second range of optical frequencies are not identical; and obtaining the signals generated by the first and second labeled primers by extracting fluorescence intensities from the first and second fluorescent images of the cluster. In some examples, the first range of optical frequencies and the second range of optical frequencies may partially overlap. For example, the first range of optical frequencies may be 500-580 THz, and the second range of optical frequencies may be 540-620 THz.

In some embodiments, the disclosed method further includes extracting fluorescence intensities from the first and second fluorescent images of the same region or substantially overlapping regions of the substrate. In some embodiments, sequence information is determined based on a combination of the extracted fluorescence intensities from the first and second fluorescent images. In some embodiments, one of a plurality of classifications is selected, based on the combination of the extracted fluorescence intensities and predetermined fluorescence intensity distributions, the plurality being greater than four. The classifications may be classifications indicating that the first and second nucleobases are both C, T, A or G, and at least one other classification that represents more than one possible combination of C, T, A and G for the first and second nucleobases. As described above, while the at least one other classification may not directly indicate a base associated with the first and second nucleobase, the at least one other classification provides information associated with the first and second nucleobases such as methylation status. In some embodiments the number of classifications is nine. In some embodiments, the disclosed method further includes: normalizing the extracted fluorescence intensities; and selecting one of the plurality of classifications, based on a combination of the normalized extracted fluorescence intensities and predetermined normalized fluorescence intensity distributions.

In some embodiments, the disclosed method further includes stimulating fluorescent emissions from the first labeled primers and second labeled primers in the cluster with light at a predetermined optical frequency. In some embodiments, the disclosed method further includes stimulating fluorescent emissions from the first labeled primers and second labeled primers in the cluster with light at two predetermined optical frequencies.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

It is to be understood that any features of the systems disclosed herein may be combined together in any desirable manner and/or configuration. Further, it is to be understood that any features of the methods disclosed herein may be combined together in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a block diagram which schematically illustrates an example sequencing system that may be used to perform the disclosed methods.

FIG. 2 shows a block diagram which schematically illustrates an example imaging system that may be used in conjunction with the example sequencing system of FIG. 1.

FIG. 3 shows a functional block diagram of an example computer system that may be used in the example sequencing system of FIG. 1.

FIG. 4A and FIG. 4B schematically illustrate nucleic acid clusters comprising two or more polynucleotide sequence portions for sequencing by the present methods.

FIG. 5A and FIG. 5B are chart which show example dye labeling schemes that may be used in conjunction with the present methods.

FIG. 6 is a plot showing graphical representations of nine distributions of signals from a nucleic acid cluster according to one embodiment of the disclosed technology.

FIG. 7 schematically illustrates how library preparation errors can obscure true variants in NGS methods.

FIG. 8 schematically illustrates the use of unique molecular indices (UMIs) for eliminating library preparation errors.

FIG. 9 is a plot showing graphical representations of nine distributions of signals from a nucleic acid cluster according to one embodiment of the disclosed technology, highlighting distributions that may be associated with library preparation errors.

FIG. 10 shows schematically how library preparation errors and true variants can be associated with different distributions of combined signal intensities.

FIG. 11 shows the effect of cytosine to uracil conversion treatment of a double-stranded polynucleotide, and a scatter plot showing the resulting distributions of signals from a nucleic acid cluster comprising the converted forward and reverse complement strands.

FIG. 12 shows the effect of methylcytosine to thymine conversion treatment of a double-stranded polynucleotide, and a scatter plot showing the resulting distributions of signals from a nucleic acid cluster comprising the converted forward and reverse complement strands.

FIGS. 13-15 are plots showing alternative signal distributions resulting from methods using different dye-encoding schemes.

FIG. 16 is a flow diagram showing a method for determining sequence information according to one embodiment of the disclosed technology.

FIG. 17 is a flow diagram showing a method for generating signals for use in the method of shown in FIG. 16.

FIG. 18 shows a forward strand, reverse strand, forward complement strand, and reverse complement strand of a polynucleotide molecule.

FIG. 19 shows the preparation of a concatenated polynucleotide sequence comprising a first portion and a second portion using a tandem insert method, comprising (A) preparation of a desired first (forked) adaptor and second (forked) adaptor from three oligos; (B) different types of first (forked) adaptors and second (forked) adaptors that do not anneal to each other due to the presence of a third oligo on at least one of the first (forked) adaptor and/or the second (forked) adaptor; (C) ligation of the template polynucleotide strand and adaptors generates three products, with the desired product containing both types of adaptor being produced at a proportion of 50%; (D) synthesis of concatenated strands from the desired product; and (E) completion of the synthesis of the concatenated strands from the desired product.

FIG. 20 shows an example of a concatenated polynucleotide sequence comprising a first portion and a second portion, as well as terminal and internal adaptor sequences.

FIG. 21 shows an example of a concatenated polynucleotide sequence comprising a first portion and a second portion, as well as terminal and internal adaptor sequences.

FIG. 22 shows a typical solid support.

FIG. 23 shows the stages of bridge amplification for concatenated polynucleotide sequences and the generation of an amplified cluster, comprising (A) a concatenated library strand hybridising to a immobilised primer; (B) generation of a template strand from the library strand; (C) dehybridisation and washing away the library strand; (D) generation of a template complement strand from the template strand via bridge amplification and dehybridisation of the sequence bridge; (E) further amplification to provide a plurality of template and template complement strands; and (F) cleavage of one set of the template and template complement strands.

FIG. 24A shows the effect of pre-treatment of library strands using C to U conversion on bases in template strands. FIG. 24B shows the effect of pre-treatment of library strands using mC to T conversion on bases in template strands.

FIG. 25 shows the steps involved in a loop fork method.

FIG. 26 shows an example of a polynucleotide sequence prepared using a loop fork method.

FIG. 27 shows an example of a polynucleotide sequence prepared using a loop fork method.

FIG. 28 shows a typical solid support.

FIG. 29 shows the stages of bridge amplification for polynucleotide templates prepared using a loop fork method and the generation of an amplified cluster, comprising (A) a concatenated library strand hybridising to a immobilised primer; (B) generation of a template strand from the library strand; (C) dehybridisation and washing away the library strand; (D) generation of a template complement strand from the template strand via bridge amplification and dehybridisation of the sequence bridge; and (E) further amplification to provide a plurality of template and template complement strands.

FIG. 30 shows a nicking strategy and subsequent sequencing using standard SBS and double stranded SBS (strand displacement SBS).

FIG. 31 shows a nicking strategy and subsequent sequencing using double stranded SBS (strand displacement SBS).

FIG. 32 shows sequencing using sequencing primers.

FIG. 33 shows a method of conducting paired-end reads.

FIG. 34 shows a method of conducting paired-end reads.

FIG. 35 shows an example of PCR stitching. Here, two sequences—a strand of a human library and a strand of a phiX library are joined together to create a single polynucleotide strand comprising both a first portion (comprising the strand of the human sequence) and a second portion (comprising the strand of the phiX sequence), as well as terminal and internal adaptor sequences.

FIG. 36 shows 9 QaM analysis conducted on the signals obtained from the custom second hyb run of Example 1. The x-axis shows signal intensity from a “red” wavelength channel, whilst the y-axis shows signal intensity from a “green” wavelength channel. G is not associated with any dyes and as such appears contributes no intensity for both “red” and “green” channels. C is associated with a “red” dye and as such contributes intensity to the “red” channel, but not the “green” channel. Tis associated with a “green” dye and as such contributes intensity to the “green” channel, but not the “red channel. A is associated with both a “red” dye and a “green” dye, and as such contributes intensity to both the “red” channel and “green” channel. Since the template comprises forward and reverse complement strands that are sequenced simultaneously, most of the readout will generate (G,G) read (bottom left corner), (C,C) read (bottom right corner), (T,T) read (top left corner), and (A,A) read (top right corner) clouds. However, a central cloud corresponding to (C,T) or (T,C) reads corresponds with the presence of modified cytosines. FIG. 36B shows sequence data generated from two different primers used (HYB2′-ME and HP10) in the custom second hyb run of Example 1. Mismatches between the two sequences allow identification of modified cytosines. For example, 5-mC present in the original forward strand of the target polynucleotide is read as Tin the HP10 read, whereas C present in the original reverse complement strand of the target polynucleotide (corresponding to the same position as 5-mC in the original forward strand of the target polynucleotide) is read as C in the HYB2′-ME read.

FIGS. 37A to 37F show 9 QaM analysis conducted on the signals obtained from Example 2 (library fragments 1 to 6). The x-axis shows signal intensity from a “red” wavelength channel, whilst the y-axis shows signal intensity from a “green” wavelength channel. A CA dye swap has been performed in this MiniSeq run compared to a standard MiniSeq run. G is not associated with any dyes and as such appears contributes no intensity for both “red” and “green” channels. A is associated with a “red” dye and as such contributes intensity to the “red” channel, but not the “green” channel. T is associated with a “green” dye and as such contributes intensity to the “green” channel, but not the “red channel. C is associated with both a “red” dye and a “green” dye, and as such contributes intensity to both the “red” channel and “green” channel. Since the template comprises forward and reverse complement strands that are sequenced simultaneously, the readout will generate (T,T) reads (top left corner), (T,C) reads (top middle), (C,C) reads (top right coiner), (G,G) reads (bottom left corner), (G,A) reads (bottom middle), and (A,A) reads (bottom right corner). The top right corner corresponds to a (5-mC)-G base pair, whilst the bottom left corner corresponds to a G-(5-mC) base pair, thus corresponding with the presence of modified cytosines. Groupings are as follows: T in forward strand of library in top left (marked as “T”); C in forward strand of library in top middle (marked as “C”); 5-mC in forward strand of library in top right (marked as “c”); G in forward strand of library and associated with 5-mC in reverse strand of library in bottom left (marked as “g”); G in forward strand of library and associated with C in reverse strand of library in bottom middle (marked as “G”); and A in forward strand of library in bottom right (marked as “A”). In FIGS. 37A to 37C, two scatter-plots are shown: the plot marked “read-color coded” corresponds to assignments for each base to particular groups during the read process; the plot marked “ref-color coded” shows the true assignments for each base to particular groups and is indicative of where errors have occurred in the read process. FIGS. 37D to 37F show combined “read-color coded” and “ref-color coded” plots—where the read and the reference differ, a border is shown for the read assignment, whilst the central portion of the circle shows the actual assignment. In addition, FIGS. 37A to 37F show sequence alignment of the read sequence to the true methylated pUC19 sample—“m” above or below a C represents 5-mC, whilst “m” above or below a G represents G that is base-paired with 5-mC; red boxes indicate errors in read (of sequence or methylation status).

FIG. 38A shows the sequencing primer binding modes used in Example 3—Read 1 (control) is conducted using only a single sequencing primer type (HP21 mix), Read 2 (control) is conducted using a single sequencing primer type (HYB2′-ME), and Read 3 is conducted using two sequencing primer types (HP10 mix and HYB2′-ME) to enable concurrent sequencing to generate a 9 QaM signal. FIG. 38B shows the results from the Read 1, Read 2 and Read 3 runs in Example 3. The plot is arranged so that G is disposed on the bottom left corner, C is disposed on the top left corner, T is disposed on the bottom right corner, and A is disposed on the top right corner. The Read 1 plot has a T base call for one of the reads (highlighted as a circled point). The Read 2 plot has a C base call for the read corresponding to the same position (highlighted as a circled point). The Read 3 plot contains (G,G) reads at the bottom left corner, (C,C) reads at the top left corner, (T,T) reads at the bottom right corner, and (A,A) reads at the top right corner. An mismatched base pair error was detected due to the presence of a (C,T) read in the central middle portion of the plot.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### INTRODUCTION

Typically, if two differing sequences are sensed in the same sampling area during NGS (e.g. in the same cluster), it is not possible to determine sequencing information from the two sequences. Therefore, according to prior methods, sequencing data from clusters comprising more than one sensed polynucleotide sequence portion (“polyclonal clusters”) are filtered out and are excluded from the sequencing output. The present methods, however, allow for sequence information to be determined from two or more sequence portions of interest simultaneously.

In some embodiments, the disclosed method enables the determination of sequence information from two or more polynucleotide sequence portions without the need for the signals generated from the different portions to be separately detectable given the configuration of the portions and the sequencing equipment. It is therefore possible to simultaneously determine sequence information from multiple polynucleotide sequence portions that are not possible to spatially resolve, for example sequencing two polynucleotide sequence portions from a signal detected at a single sensing region (for example a single pixel of an imaging sensor) and/or from a signal obtained from a single cluster (i.e. a single contiguous cluster containing both of the two or more sequence portions), thus increasing the efficiency of the sequencing workflow.

At any given position, the nucleobases present in the two or more different polynucleotide sequence portions may be the same or different. The sequence information may, therefore, be indicative of matches or mismatches between the different portions. Where the nucleobases present in the two or more different sequence portions are the same at a given position, the sequence information may be indicative of the identity of the nucleobase at that position (A, T, C or G). Additionally or alternatively, a priori knowledge (e.g. of the library preparation methods used) can be leveraged to provide additional useful information, for example relating to a sequence from which the different sequence portions are derived. In some instances the present methods allow for the sequence of a double-stranded DNA target molecule, including methylation status, to be determined in a single sequencing run, using signals from two or more polynucleotide sequence portions derived from the double-stranded molecule.

In some embodiments, the primer for sequencing a first sequence portion and the primer for sequencing a second sequence portion are annealed/hybridized to the molecules in the same reaction step to reduce chemical reaction steps, thus saving time and increasing the efficiency of sequencing-by-synthesis (SBS) workflows. Then, both sequence portions may be read-out through SBS chemistry cycles in the same reaction run.

In some embodiments, the disclosed technology comprises obtaining sequence information using Illumina's sequencing-by-synthesis and reversible terminator-based sequencing chemistry with removable fluorescent dyes (e.g., as described in Bentley et al., Nature 6:53-59 [2009]). Short sequence reads of about tens to a few hundred base pairs may be aligned against a reference genome and unique mapping of the short sequence reads to the reference genome may be identified. Further details regarding the sequencing-by-synthesis and dye labeling methods which can be used by the disclosed technology are described in U.S. Patent Application Publication Numbers 2007/0166705, 2006/0188901, 2006/0240439, 2006/0281109, 2005/0100900, 2013/0079232, U.S. Pat. No. 7,057,026, PCT Application Publication Numbers WO 2005/065814, WO 2006/064199, WO 2007/010251, and WO 2018/165099. U.S. patent application Ser. No. 17/338,590, U.S. Pat. Nos. 7,601,499, 9,267,173, and U.S. Patent Publication No. 2012/0053063, the disclosures of which are incorporated herein by reference in their entireties.

### Example Sequencer

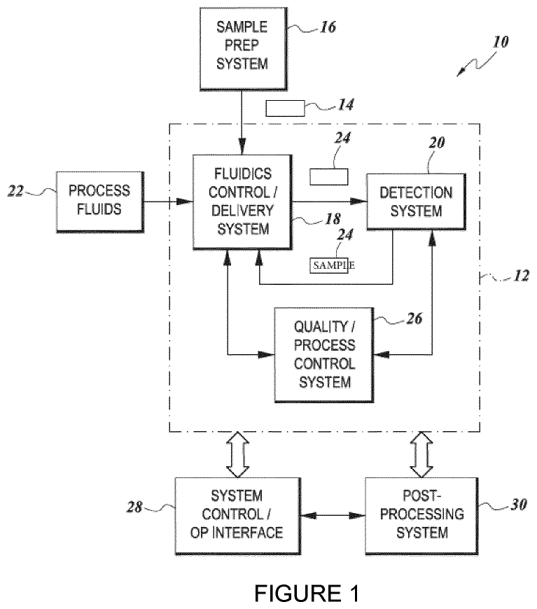

Referring to FIG. 1, a diagrammatical representation of an example sequencing system 10 is illustrated as including a sequencer 12 designed to determine sequences of genetic material of a sample 14. The sequencer may function in a variety of manners, and based upon a variety of techniques, including sequencing by primer extension using labeled nucleotides, as in a presently contemplated embodiment, as well as other sequencing techniques such as sequencing by ligation or pyrosequencing. In some embodiments, the sequencer 12 progressively moves samples through reaction cycles and imaging cycles to progressively build oligonucleotides by binding nucleotides to templates at individual sites on the sample. In some embodiments, the sample may be prepared by a sample preparation system 16. This process may include amplification of fragments of DNA or RNA on a support to create a multitude of sites of DNA or RNA fragments the sequence of which are determined by the sequencing process. Exemplary methods for producing sites of amplified nucleic acids suitable for sequencing include, but are not limited to, rolling circle amplification (RCA) (Lizardi et al., Nat. Genet. 19:225-232 (1998)), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11 (2005); or U.S. Pat. No. 5,641,658), polony generation (Mitra et al., Proc. Natl. Acad. Sci. USA 100:5926-5931 (2003); Mitra et al., Anal. Biochem. 320:55-85 (2003)), or clonal amplification on beads using emulsions (Dressman et al., Proc. Natl. Acad. Sci. USA 100:8817-8822 (2003)) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002)), each of the aforementioned publications is incorporated herein by reference. The sample preparation system 16 may dispose the sample, which may be in the form of an array of sites, in a sample container for processing and imaging.

In some embodiments, the sequencer 12 includes a fluidics control/delivery system 18 and a detection system 20. The fluidics control/delivery system 18 may receive a plurality of process fluids as indicated by reference numeral 22, for circulation through the sample containers of the samples in process, designated by reference numeral 24. As will be appreciated by those skilled in the art, the process fluids may vary depending upon the particular stage of sequencing. For example, in sequencing-by-synthesis (SBS) using labeled nucleotides, the process fluids introduced to the sample may include a polymerase and tagged nucleotides of the four common DNA types, each nucleotide having a unique fluorescent tag and a blocking agent linked to it. The fluorescent tag allows the detection system 20 to detect which nucleotides were last added to primers hybridized to template nucleic acids at individual sites in the array, and the blocking agent prevents addition of more than one nucleotide per cycle at each site.

At other phases of the sequencing cycles, the process fluids 22 may include other fluids and reagents, such as reagents for removing extension blocks from nucleotides or cleaving nucleotide linkers to release a newly extendable primer terminus. For example, once reactions have taken place at individual sites in the array of the samples, the initial process fluid containing the tagged nucleotides may be washed from the sample in one or more flushing operations. The sample may then undergo detection, such as by the optical imaging at the detection system 20. Subsequently, reagents may be added by the fluidics control/delivery system 18 to de-block the last added nucleotide and remove the fluorescent tag from each. The fluidics control/delivery system 18 may then again wash the sample, which is then prepared for a subsequent cycle of sequencing. Exemplary fluidic and detection configurations that can be used in the methods and devices set forth herein are described in WO 07/123744, which is incorporated herein by reference. In some embodiments, such sequencing may continue until the quality of data derived from sequencing degrades due to cumulative loss of yield or until a predetermined number of cycles have been completed.

In some embodiments, the quality of samples 24 in process as well as the quality of the data derived by the system, and the various parameters used for processing the samples is controlled by a quality/process control system 26. The quality/process control system 26 may include one or more programmed processors, or general purpose or application-specific computers which communicate with sensors and other processing systems within the fluidics control/delivery system 18 and the detection system 20. A number of process parameters may be used for sophisticated quality and process control, for example, as part of a feedback loop that can change instrument operation parameters during the course of a sequencing run.

In some embodiments, the sequencer 12 also communicates with a system control/operator interface 28 and ultimately with a post-processing system 30. The system control/operator interface 28 may include a general purpose or application-specific computer designed to monitor process parameters, acquired data, system settings, and so forth. The operator interface may be generated by a program executed locally or by programs executed within the sequencer 12. In some embodiments, these may provide visual indications of the health of the systems or subsystems of the sequencer, the quality of the data acquired, and so forth. The system control/operator interface 28 may also permit human operators to interface with the system to regulate operation, initiate and interrupt sequencing, and any other interactions that may be desired with the system hardware or software. For instance, the system control/operator interface 28 may automatically undertake and/or modify steps to be performed in a sequencing procedure, without input from a human operator. Alternatively or additionally, the system control/operator interface 28 may generate recommendations regarding steps to be performed in a sequencing procedure and display these recommendations to the human operator. This mode may allow for input from the human operator before undertaking and/or modifying steps in the sequencing procedure. In addition, the system control/operator interface 28 may provide an option to the human operator allowing the human operator to select certain steps in a sequencing procedure to be automatically performed by the sequencer 12 while requiring input from the human operator before undertaking and/or modifying other steps. In any event, allowing both automated and operator interactive modes may provide increased flexibility in performing the sequencing procedure. In addition, the combination of automation and human-controlled interaction may further allow for a system capable of creating and modifying new sequencing procedures and algorithms through adaptive machine learning based on the inputs gathered from human operators.

The post-processing system 30 may further include one or more programmed computers that receive detected information, which may be in the form of pixilated image data and derive sequence data from the image data. The post-processing system 30 may include image recognition algorithms which distinguish between colors of dyes (e.g., fluorescent emission spectra of dyes) attached to nucleotides that bind at individual sites as sequencing progresses (e.g., by analysis of the image data encoding specific colors and/or intensities), and logs the sequence of the nucleotides at the individual site locations. Progressively, then, the post-processing system 30 may build sequence lists for the individual sites of the sample array which can be further processed to establish genetic information for extended lengths of material by various bioinformatics algorithms.

The sequencing system 10 may be configured to handle individual samples or may be designed for higher throughput in a manner in which multiple stations are provided for the delivery of reagents and other fluids, and for detection of progressively building sequences of nucleotides. Further details can be found in U.S. Pat. No. 9,797,012, which is incorporated herein by reference.

Samples may be removed from processing, reprocessed, and scheduling of such processing may be altered in real time, particularly where the fluidics control system 18 or the quality/process control system 26 detect that one or more operations were not performed in an optimal or desired manner. In embodiments wherein a sample is removed from the process or experiences a pause in processing that is of a substantial duration, the sample can be placed in a storage state. Placing the sample in a storage state can include altering the environment of the sample or the composition of the sample to stabilize biomolecule reagents, biopolymers or other components of the sample.

Exemplary methods for altering the sample environment include, but are not limited to, reducing temperature to stabilize sample constituents, addition of an inert gas to reduce oxidation of sample constituents, and removing from a light source to reduce photobleaching or photodegradation of sample constituents. Exemplary methods of altering sample composition include, without limitation, adding stabilizing solvents such as antioxidants, glycerol and the like, altering pH to a level that stabilizes enzymes, or removing constituents that degrade or alter other constituents. In addition, certain steps in the sequencing procedure may be performed before removing the sample from processing. For instance, if it is determined that the sample should be removed from processing, the sample may be directed to the fluidics control/delivery system 18 so that the sample may be washed before storage. These steps may be taken to ensure that no information from the sample is lost.

Moreover, sequencing operations may be interrupted by the sequencer 12 at any time upon the occurrence of certain predetermined events. These events may include, without limitation, unacceptable environmental factors such as undesirable temperature, humidity, vibrations or stray light; inadequate reagent delivery or hybridization; unacceptable changes in sample temperature; unacceptable sample site number/quality/distribution; decayed signal-to-noise ratio; insufficient image data; and so forth. It should be noted that the occurrence of such events need not require interruption of sequencing operations. Rather, such events may be factors weighed by the quality/process control system 26 in determining whether sequencing operations should continue. For example, if an image of a particular cycle is analyzed in real time and shows a low signal for that optical channel, the image can be re-exposed using a longer exposure time, or have a particular chemical treatment repeated. If the image shows a bubble in a flow cell, the instrument can automatically flush more reagent to remove the bubble, then re-record the image. If the image shows low signal for a particular optical channel in one cycle due to a fluidics problem, the instrument can automatically halt scanning and reagent delivery for that particular optical channel, thus saving on analysis time and reagent consumption.

Although the system has been exemplified above with regard to a system in which a sample interfaces with different stations by physical movement of the sample, it will be understood that the principles set forth herein are also applicable to a system in which the steps occurring at each station are achieved by other means not requiring movement of the sample. For example, reagents present at the stations can be delivered to a sample by means of a fluidic system connected to reservoirs containing the various reagents. Similarly, an optics system can be configured to detect a sample that is in fluid communication with one or more reagent stations. Thus, detection steps can be carried out before, during or after delivery of any particular reagent described herein. Accordingly, samples can be effectively removed from processing by discontinuing one or more processing steps, be it fluid delivery or optical detection, without necessarily physically removing the sample from its location in the device.

Disclosed systems can be used to continuously sequence nucleic acids in a plurality of different samples. Disclosed systems can be configured to include an arrangement of samples and an arrangement of stations for carrying out sequencing steps. The samples in the arrangement of samples can be placed in a fixed order and at fixed intervals relative to each other. For example, an arrangement of nucleic acid arrays can be placed along the outer edge of a circular table. Similarly, the stations can be placed in a fixed order and at fixed intervals relative to each other. For example, the stations can be placed in a circular arrangement having a perimeter that corresponds to the layout for the arrangement of sample arrays. Each of the stations can be configured to carry out a different manipulation in a sequencing protocol. The arrangements of sample arrays and stations can be moved relative to each other such that the stations carry out desired steps of a reaction scheme at each reaction site. The relative locations of the stations and the schedule for the relative movement can correlate with the order and duration of reaction steps in the sequencing reaction scheme such that once a sample array has completed a cycle of interacting with the full set of stations, then a single sequencing reaction cycle is complete. For example, primers that are hybridized to nucleic acid targets on an array can each be extended by addition of a single nucleotide, detected and de-blocked if the order of the stations, spacing between the stations, and rate of passage for the array corresponds to the order of reagent delivery and reaction time for a complete sequencing reaction cycle.

In accordance with the configuration set forth above, each lap (or full revolution in embodiments where a circular table is used) completed by an individual sample array can correspond to determination of a single nucleotide for each of the target nucleic acids on the array (e.g., including the steps of incorporation, imaging, cleavage and de-blocking carried out in each cycle of a sequencing run). Furthermore, several sample arrays present in the system (for example, on the circular table) concurrently move along similar, repeated laps through the system, thereby resulting in continuous sequencing by the system. Using the disclosed systems or methods, reagents can be actively delivered or removed from a first sample array in accordance with a first reaction step of a sequencing cycle while incubation, or some other reaction step in the cycle, occurs for a second sample array. Thus, a set of stations can be configured in a spatial and temporal relationship with an arrangement of sample arrays such that reactions occur at multiple sample arrays concurrently even as the sample arrays are subjected to different steps of the sequencing cycle at any given time, thereby allowing continuous and simultaneous sequencing to be performed. Such a circular system may be used when the chemistry and imaging times are disproportionate. For small flow cells that only take a short time to scan, the system may have a number of flow cells running in parallel in order to optimize the time the instrument spends acquiring data. When the imaging time and chemistry time are equal, a system that is sequencing a sample on a single flow cell spends half the time performing a chemistry cycle rather than an imaging cycle, and therefore a system that can process two flow cells could have one on the chemistry cycle and one on the imaging cycle. When the imaging time is ten-fold less than the chemistry time, the system can have ten flow cells at various stages of the chemistry process whilst continually acquiring data.

In some embodiments, the disclosed system is configured to allow replacement of a first sample array with a second sample array while the system continuously sequences nucleic acids of a third sample array. Thus, a first sample array can be individually added or removed from the system without interrupting sequencing reactions occurring at another sample array, thereby allowing continuous sequencing for the set of sample arrays. Moreover, sequencing runs of different lengths can be performed continuously and simultaneously in the system because individual sample arrays can complete a different number of laps through the system and the sample arrays can be removed or added to the system in an independent fashion such that reactions occurring at other sites are not perturbed.

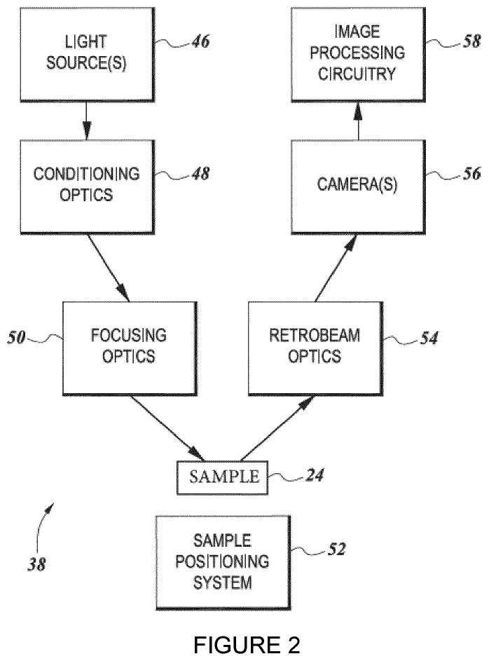

FIG. 2 illustrates an exemplary detection station 38 which can detect nucleotides added at sites of an array and can be used in conjunction with the example sequencing system of FIG. 1. As set forth above, a sample can be moved to two or more stations of the device that are located in physically different locations or alternatively one or more steps can be carried out on a sample that is in communication with the one or more stations without necessarily being moved to different locations. Accordingly, the description herein with regard to particular stations is understood to relate to stations in a variety of configurations whether or not the sample moves between stations, the stations move to the sample, or the stations and sample are static with respect to each other. In the embodiment illustrated in FIG. 2, one or more light sources 46 provide light beams that are directed to conditioning optics 48. The light sources 46 may include one or more lasers, with multiple lasers being used for detecting dyes that fluoresce at different corresponding wavelengths. The light sources may direct beams to the conditioning optics 48 for filtering and shaping of the beams in the conditioning optics.

For example, in a presently contemplated embodiment, the conditioning optics 48 combine beams from multiple lasers and generate a substantially linear beam of radiation that is conveyed to focusing optics 50. The laser modules can additionally include a measuring component that records the power of each laser. The measurement of power may be used as a feedback mechanism to control the length of time an image is recorded in order to obtain a uniform exposure energy, and therefore signal, for each image. If the measuring component detects a failure of the laser module, then the instrument can flush the sample with a “holding buffer” to preserve the sample until the error in the laser can be corrected.

The sample 24 is positioned on a sample positioning system 52 that may appropriately position the sample in three dimensions, and may displace the sample for progressive imaging of sites on the sample array. In a presently contemplated embodiment, the focusing optics 50 confocally direct radiation to one or more surfaces of the array at which individual sites are located that are to be sequenced. Depending upon the wavelengths of light in the focused beam, a retrobeam of radiation is returned from the sample due to fluorescence of dyes bound to the nucleotides at each site.

The retrobeam is then returned through retrobeam optics 54 which may filter the beam, such as to separate different wavelengths in the beam, and direct these separated beams to one or more cameras 56. The cameras 56 may be based upon any suitable technology, such as including charge coupled devices that generate pixilated image data based upon photons impacting locations in the devices. In some embodiments, the cameras 56 may include CMOS sensors. In some embodiments, the cameras 56 may include one or more point-and-shoot cameras. In some embodiments, the cameras 56 may include one or more time delay and integration (TOI) cameras. The cameras generate image data that is then forwarded to image processing circuitry 58. In some embodiments, the processing circuitry 58 may perform various operations, such as analog-to-digital conversion, scaling, filtering, and association of the data in multiple frames to appropriately and accurately image multiple sites at specific locations on the sample. The image processing circuitry 58 may store the image data, and may ultimately forward the image data to the post-processing system 30 where sequence data can be derived from the image data. Example detection devices that can be used at a detection station include, for example, those described in US 2007/0114362 (U.S. patent application Ser. No. 11/286,309) and WO 07/123744, each of which is incorporated herein by reference.

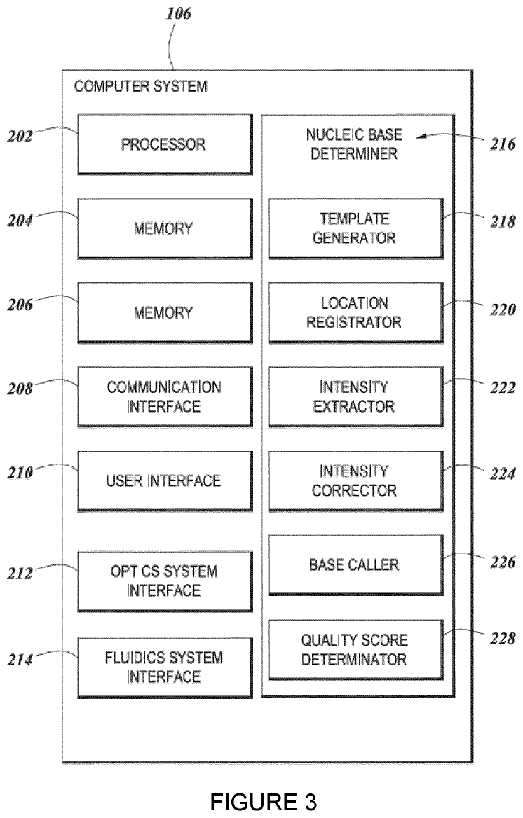

A computer system 106 as illustrated in FIG. 3 may be used to implement the system control/operator interface 28 and the post-processing system 30 of the example sequencing system 10 in FIG. 1. As shown in FIG. 3, the computer system 106 can include functionalities for controlling optics/fluidics systems and determining nucleobase sequences of polynucleotides.

In one embodiment, the computer system 106 includes a processor 202 that is in electrical communication with a memory 204, a storage 206, and a communication interface 208. The processor 202 can be configured to execute instructions that cause the fluidics system 104 to supply reagents to the flow cell 114 during sequencing reactions. The processor 202 can execute instructions that control the light source 120 of the optics system 102 to generate light at around a predetermined wavelength. The processor 202 can execute instructions that control the detector 126 of the optics system 102 and receive data from the detector 126. The processor 202 can execute instructions to process data, for example fluorescent images, received from the detector 126 and to determine the nucleotide sequences of polynucleotides based on the data received form the detector 126. The memory 204 can be configured to store instructions for configuring the processor 202 to perform the functions of the computer system 106 when the sequencing system 100 is powered on. When the sequencing system 100 is powered off, the storage 206 can store the instructions for configuring the processor 202 to perform the functions of the computer system 106. The communication interface 208 can be configured to facilitate the communications between the computer system 106, the optics system 102, and the fluidics system 104.

The computer system 106 can include a user interface 210 configured to communicate with a display device (not shown) for displaying the sequencing results of the sequencing system 100. The user interface 210 can be configured to receive inputs from users of the sequencing system 100. An optics system interface 212 and a fluidics system interface 214 of the computer system 106 can be configured to control the optics system 102 and the fluidics system 104 through communication links (not shown). For example, the optics system interface 212 can communicate with the computer interface 110 of the optics system 102 through a communication link.

The computer system 106 can include a nucleic base determiner 216 configured to determine the nucleotide sequence of polynucleotides using the data received from the detector 126. The nucleic base determiner 216 can include one or more of: a template generator 218, a location registrator 220, an intensity extractor 222, an intensity corrector 224, a base caller 226, and a quality score determiner 228. The template generator 218 can be configured to generate a template of the locations of polynucleotide clusters in the flow cell 114 using the fluorescent images captured by the detector 126. The location registrator 220 can be configured to register the locations of polynucleotide clusters in the flow cell 114 in the fluorescent images captured by the detector 126 based on the location template generated by the template generator 218. The intensity extractor 222 can be configured to extract intensities of the fluorescent emissions from the fluorescent images to generate extracted intensities. For example, the peak intensity value found in a diffraction-limited spot of a DNA cluster may be extracted from the image and used to represent the signal of the DNA cluster. For another example, the total intensity included within a diffraction-limited spot of a DNA cluster may be extracted from the image and used to represent the signal of the DNA cluster. Alternatively, the intensity estimate can be made through the use of equalization and channel estimation.

The intensity corrector 224 can be configured to reduce or eliminate noise or aberration inherent in the sequencing reaction or optical system. For example, intensity may be influenced by laser intensity fluctuation, DNA cluster shape/size variation, uneven illumination, optical distortions or aberrations, and/or phasing/pre-phasing that occur in the DNA clusters. In some embodiments, the intensity corrector 224 can phase correct or pre-phase correct extracted intensities. In some embodiments, the intensity corrector 224 can normalize extracted fluorescence intensities to reduce or eliminate the effect of DNA cluster size variation. For example, each DNA template may contain the same calibration oligonucleotide. Thus, the extracted fluorescence intensity of a cluster obtained from sequencing a known nucleotide in the calibration oligonucleotide can be used as a normalization factor for that cluster. The intensity corrector 224 can divide the extracted fluorescence intensities of that cluster obtained from sequencing nucleotides in other regions of the DNA template by the normalization factor to obtain the normalized extracted fluorescence intensities. The base caller 226 can be configured to determine the nucleobases of a polynucleotide from the corrected intensities. The bases of a polynucleotide determined by the base caller 226 can be associated with quality scores determined by the quality score determiner 228. Quality scoring refers to the process of assigning a quality score to each base call. To evaluate the quality of a base call from a sequencing read, example processes can include calculating a set of predictor values for the base call and using the predictor values to look up a quality score in a quality table. The quality score can be presented in any suitable format that allows a user to determine the probability of error of any given base call. In some embodiments, the quality score is presented as a numerical value. For example, the quality score can be quoted as QXX where the XX is the score and it means that that particular call has a probability of error of 1_{o-XXJ}¹⁰. Thus, as an example, Q30 equates to an error rate of 1 in 1000, or 0.1% and Q40 equates to an error rate of 1 in 10,000 or 0.01%. The error rate can be calculated using a control nucleic acid. Additionally, some metrics displays can include the error rate on a per-cycle basis. In some embodiments, the quality table is generated using on a calibration data set, the calibration set being representative of run and sequence variability. Further details of the computations that can be performed by the nucleic base determiner, calculation of error rate and quality score may be found in U.S. Pat. No. 8,392,126, U.S. Patent Application Publication Numbers 2020/0080142 and 2012/0020537, each of which is incorporated by reference herein in its entirety. While nucleic base determiner 216 is shown as part of computer system 106 in FIG. 3, it will be appreciated that nucleic base determiner 216 may be a separate computing device from the other components shown in FIG. 3 such that nucleic base determiner 216 may receive and process image data in a computing device that is different to a computing device that provides optics and fluidics control.

### Clusters

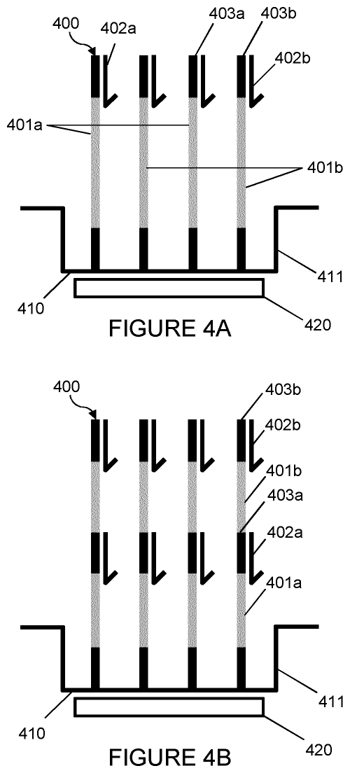

FIGS. 4A-4B each illustrate a respective plurality of polynucleotide molecules 400 comprising multiple copies of two polynucleotide sequence portions of interest 401a, 401b for processing to determine sequence information based upon a single combined signal obtained from the two polynucleotide sequence portions of interest according to the present methods. For example, the plurality of polynucleotide molecules 400 illustrated in FIGS. 4A and 4B may be configured on a substrate 410 such that light emissions from the plurality of polynucleotide molecules are detected by a single sensing portion (for example a single pixel of an imaging sensor 420). Additionally or alternatively, the plurality of polynucleotide molecules 400 may comprise a single cluster (i.e. a single contiguous cluster containing both of the two or more sequence portions 401a, 401b) such that light emissions from each of the two respective portions cannot be spatially resolved. The substrate 410 may be a flow cell, which may be patterned or unpatterned. In one example, the substrate 410 may be a patterned flow cell comprising a number of discrete nanowells 411, with each well containing polynucleotide molecules comprising two or more polynucleotide sequence portions for sequencing and each well having a single respective sensor associated with the well. Because each a single sensor is associated with the well, signals from the two or more portions of interest cannot be resolved, irrespective of whether the different portions (or respective clusters) are spatially resolved within the well. Two or more polynucleotide sequence portions of interest contained within a single well in this way is sometimes referred to herein as a “cluster” irrespective of whether the different portions are spatially resolved in the well given that light emissions from such a well form a single combined signal.

In one example, as shown in FIG. 4A, the first and second sequence portions 401a, 401b are present in different polynucleotide molecules 400. In the example shown in FIG. 4B, the first and second portions 401a, 401b are present as respective portions of the same molecules 400.

Both the first and second sequence portions 401a, 401b in the cluster can be sequenced simultaneously using first primers 402a specific to the first portion 401a, or to a region 403a adjacent to the first portion, and second primers 402b specific to the second portion 401b, or to a region 403b adjacent to the second portion, in the same reaction run. For example, the first and second sequence portions 401a, 401b may be flanked at one or both ends by respective primer binding sites 403a, 403b having a known sequence.

Sequencing primers 402a, 402b specific to the different primer binding sites 403a, 403b can therefore be designed and used for simultaneous sequencing of the two sequence portions 401a, 401b.

As described above, a single combined signal may be obtained from the two polynucleotide sequence portions of interest 401a, 401b according to the present methods.

For example, the plurality of polynucleotide molecules 400 may be configured on the flow cell 410 such that light emissions from the plurality of polynucleotide molecules are detected by a single sensing portion 420. Alternatively or additionally, the plurality of polynucleotide molecules may comprise a single cluster such that light emissions from each of the respective two polynucleotide sequence portions cannot be spatially resolved.

Since the fluorescent signal associated with the extended first portion sequencing primers 402a and the fluorescent signal associated with the extended second portion sequencing primers 402b is combined, the signals may not be optically resolved.

### Sequencing

As described herein, the template provides information (e.g. identification of the genetic sequence, identification of epigenetic modifications) on the original target polynucleotide sequence. For example, a sequencing process (e.g. a sequencing-by-synthesis or sequencing-by-ligation process) may reproduce information that was present in the original target polynucleotide sequence, by using complementary base pairing.

In one embodiment, sequencing may be carried out using any suitable “sequencing-by-synthesis” technique, wherein nucleotides are added successively in cycles to the free 3′ hydroxyl group, resulting in synthesis of a polynucleotide chain in the 5′ to 3′ direction. The nature of the nucleotide added may be determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. Such a label may be configured to emit a signal, such as an electromagnetic signal or a (visible) light signal.

In a particular embodiment, the label is a fluorescent label (e.g. a dye). Thus, such a label may be configured to emit an electromagnetic signal, such as a (visible) light signal. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

However, the detectable label need not be a fluorescent label. Any label can be used which allows the detection of the incorporation of the nucleotide into the DNA sequence.

Each cycle may involve simultaneous delivery of four different nucleotide types to the array of template molecules. Alternatively, different nucleotide types can be added sequentially and an image of the array of template molecules can be obtained between each addition step.

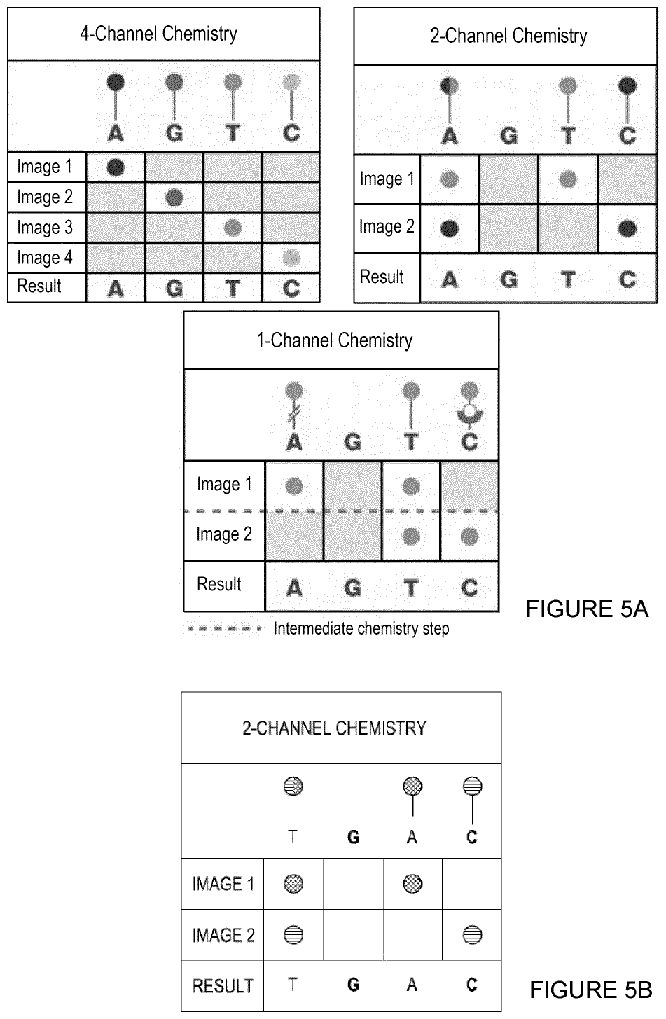

In some embodiments, each nucleotide type may have a (spectrally) distinct label. In other words, four channels may be used to detect four nucleobases (also known as 4-channel chemistry) (FIG. 5A—left). For example, a first nucleotide type (e.g. A) may include a first label (e.g. configured to emit a first wavelength, such as red light), a second nucleotide type (e.g. G) may include a second label (e.g. configured to emit a second wavelength, such as blue light), a third nucleotide type (e.g. T) may include a third label (e.g. configured to emit a third wavelength, such as green light), and a fourth nucleotide type (e.g. C) may include a fourth label (e.g. configured to emit a fourth wavelength, such as yellow light). Four images can then be obtained, each using a detection channel that is selective for one of the four different labels. For example, the first nucleotide type (e.g. A) may be detected in a first channel (e.g. configured to detect the first wavelength, such as red light), the second nucleotide type (e.g. G) may be detected in a second channel (e.g. configured to detect the second wavelength, such as blue light), the third nucleotide type (e.g. T) may be detected in a third channel (e.g. configured to detect the third wavelength, such as green light), and the fourth nucleotide type (e.g. C) may be detected in a fourth channel (e.g. configured to detect the fourth wavelength, such as yellow light).

Although specific pairings of bases to signal types (e.g. wavelengths) are described above, different signal types (e.g. wavelengths) and/or permutations may also be used.

In some embodiments, detection of each nucleotide type may be conducted using fewer than four different labels. For example, sequencing-by-synthesis may be performed using methods and systems described in US 2013/0079232, which is incorporated herein by reference.

Thus, in some embodiments, two channels may be used to detect four nucleobases (also known as 2-channel chemistry) (FIG. 5A—middle). For example, a first nucleotide type (e.g. A) may include a first label (e.g. configured to emit a first wavelength, such as green light) and a second label (e.g. configured to emit a second wavelength, such as red light), a second nucleotide type (e.g. G) may not include the first label and may not include the second label, a third nucleotide type (e.g. T) may include the first label (e.g. configured to emit the first wavelength, such as green light) and may not include the second label, and a fourth nucleotide type (e.g. C) may not include the first label and may include the second label (e.g. configured to emit the second wavelength, such as red light). Two images can then be obtained, using detection channels for the first label and the second label. For example, the first nucleotide type (e.g. A) may be detected in both a first channel (e.g. configured to detect the first wavelength, such as red light) and a second channel (e.g. configured to detect the second wavelength, such as green light), the second nucleotide type (e.g. G) may not be detected in the first channel and may not be detected in the second channel, the third nucleotide type (e.g. T) may be detected in the first channel (e.g. configured to detect the first wavelength, such as red light) and may not be detected in the second channel, and the fourth nucleotide type (e.g. C) may not be detected in the first channel and may be detected in the second channel (e.g. configured to detect the second wavelength, such as green light). Although specific pairings of bases to signal types (e.g. wavelengths) and/or combinations of channels are described above, different signal types (e.g. wavelengths) and/or permutations may also be used.

In some embodiments, one channel may be used to detect four nucleobases (also known as 1-channel chemistry) (FIG. 5A—right). For example, a first nucleotide type (e.g. A) may include a cleavable label (e.g. configured to emit a wavelength, such as green light), a second nucleotide type (e.g. G) may not include a label, a third nucleotide type (e.g. T) may include a non-cleavable label (e.g. configured to emit the wavelength, such as green light), and a fourth nucleotide type (e.g. C) may include a label-accepting site which does not include the label. A first image can then be obtained, and a subsequent treatment carried out to cleave the label attached to the first nucleotide type, and to attach the label to the label-accepting site on the fourth nucleotide type. A second image may then be obtained. For example, the first nucleotide type (e.g. A) may be detected in a channel (e.g. configured to detect the wavelength, such as green light) in the first image and not detected in the channel in the second image, the second nucleotide type (e.g. G) may not be detected in the channel in the first image and may not be detected in the channel in the second image, the third nucleotide type (e.g. T) may be detected in the channel (e.g. configured to detect the wavelength, such as green light) in the first image and may be detected in the channel (e.g. configured to detect the wavelength, such as green light) in the second image, and the fourth nucleotide type (e.g. C) may not be detected in the channel in the first image and may be detected in the channel in the second image (e.g. configured to detect the wavelength, such as green light). Although specific pairings of bases to signal types (e.g. wavelengths) and/or combinations of images are described above, different signal types (e.g. wavelengths), images and/or permutations may also be used.

In some embodiments, the fluorescent labels are selected from the group consisting of polymethine derivatives, coumarin derivatives, benzopyran derivatives, chromenoquinoline derivatives, compounds containing bis-boron heterocycles such as BOPPY and BOPYPY. In some embodiments, the fluorescent label is attached to the nucleotide through a cleavable linker. In some further embodiments, the labeled nucleotide may have the fluorescent label attached to the CS position of a pyrimidine base or the C7 position of a 7-deaza purine base, optionally through a cleavable linker moiety. For example, the nucleobase may be 7-deaza adenine and the dye is attached to the 7-deaza adenine at the C7 position, optionally through a cleavable linker. The nucleobase may be 7-deaza guanine and the dye is attached to the 7-deaza guanine at the C7 position, optionally through a cleavable linker. The nucleobase may be cytosine and the dye is attached to the cytosine at the CS position, optionally through a cleavable linker. As another example, the nucleobase may be thymine or uracil and the dye is attached to the thymine or uracil at the CS position, optionally through a cleavable linker. In some further embodiments, the cleavable linker may comprise similar or the same chemical moiety as the reversible terminator 3′ hydroxy blocking group such that the 3′ hydroxy blocking group and the cleavable linker may be removed under the same reaction condition or in a single chemical reaction. Non-limiting example of the cleavable linker include the LN3 linker, the sPA linker, and the AOL linker, each of which is exemplified below.

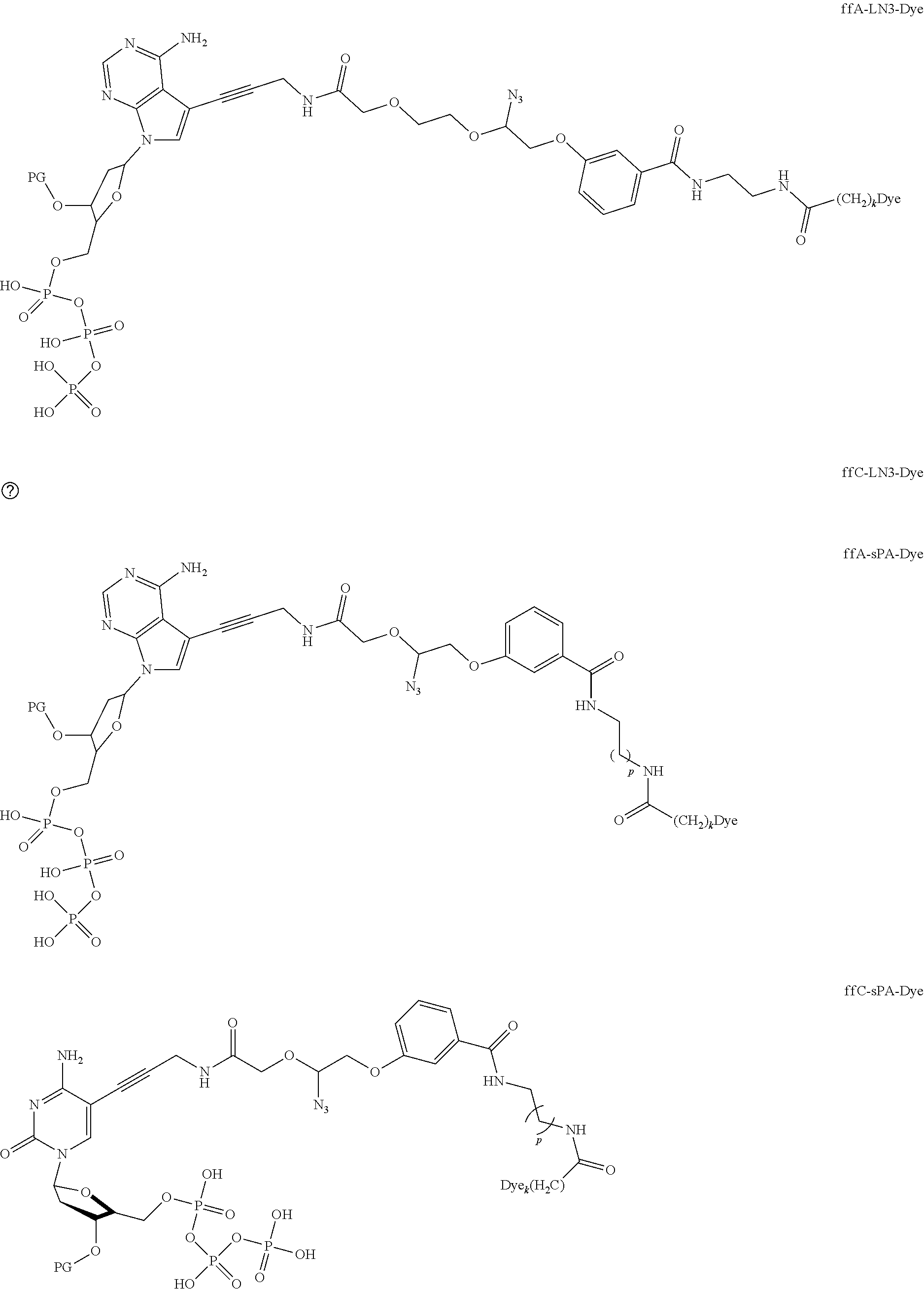

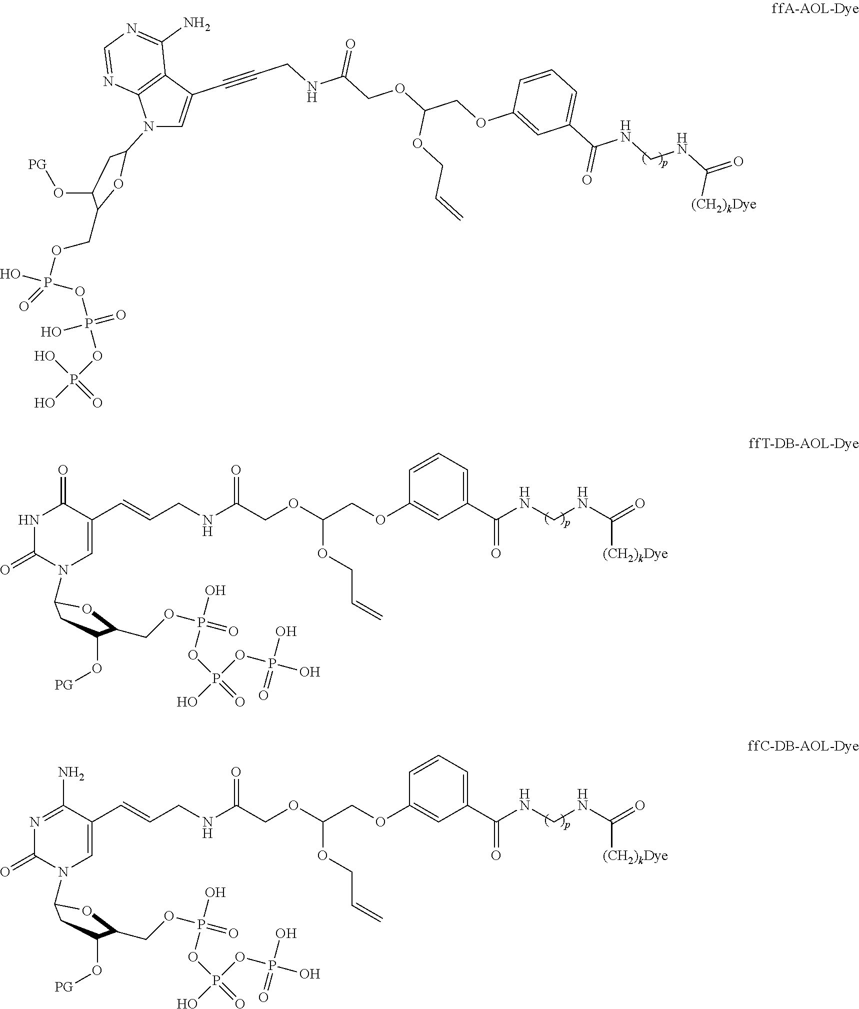

In some embodiments, the nucleotides are selected from the group consisting of an analog of dGTP, an analog of dTTP, an analog of dUTP, an analog of dCTP, and an analog of dATP. In some embodiments, the first nucleotide is a first reversibly blocked nucleotide triphosphate (rbNTP), the second nucleotide is a second rbNTP, the third nucleotide is a third rbNTP, and the fourth nucleotide is a fourth rbNTP, wherein each of the first nucleotide, second nucleotide, third nucleotide and fourth nucleotide is a different type of nucleotide from the other. In some embodiments, the four rbNTPs are selected from the group consisting of rbATP, rbTTP, rbUTP, rbCTP, and rbGTP. In some embodiments, each of the four rbNTPs includes a modified base and a reversible terminator 3′ blocking group. Non-limiting example of the 3′ blocking group include azidomethyl (*—CH2N3), substituted azidomethyl (e.g., *—CH(CHF2)N3 or *—CH(CH2F)N3) and —CH2-O—CH2—CH═CH2, where the asterisk * indicates the point attachment to the 3′ oxygen of the ribose or deoxyribose ring of the nucleotide.

Further details about the dyes and the fully functionalized nucleotides can be found in U.S. Patent Application Publication Numbers 2018/0094140 and 2020/0277670, International Patent Application Publication Number 2017/051201, and U.S. Provisional Patent Application Nos. 63/057,758 and 63/127,061, the disclosures of which are incorporated herein by reference in their entireties.

### Signal Processing

For a cluster comprising two different polynucleotide sequence portions (e.g. as shown in FIGS. 4A and 4B), there are sixteen possible combinations of nucleobases at any given position (i.e., an A in the first sequence portion and an A in the second sequence portion, an A in the first sequence portion and a T in the second sequence portion, and soon). When the same nucleobase is present at a given position in both sequence portions, the light emissions associated with each portion during the relevant base calling cycle will be characteristic of the same nucleobase. In effect, the cluster behaves as a cluster containing only one sensed sequence portion, and the identity of the bases at that position are uniquely callable.

However, when a nucleobase of the first polynucleotide sequence portion is different from a nucleobase at a corresponding position of the second polynucleotide sequence portion, the light emissions associated with each portion in the relevant base calling cycle will be characteristic of different nucleobases. In one embodiment, the fluorescent signal coming from the collection of extended first portion sequencing primers 402a have substantially the same intensity as the fluorescent signal coming from the collection of extended second portion sequencing primers 402b in the same cluster. The two signals may also be co-localized, and may not be optically resolved. Therefore, when different nucleobases are present at corresponding positions of the sequence portions, the identity of the nucleobases cannot be uniquely called from the combined signal alone. However, it is demonstrated herein that useful sequencing information can still be determined from these signals.

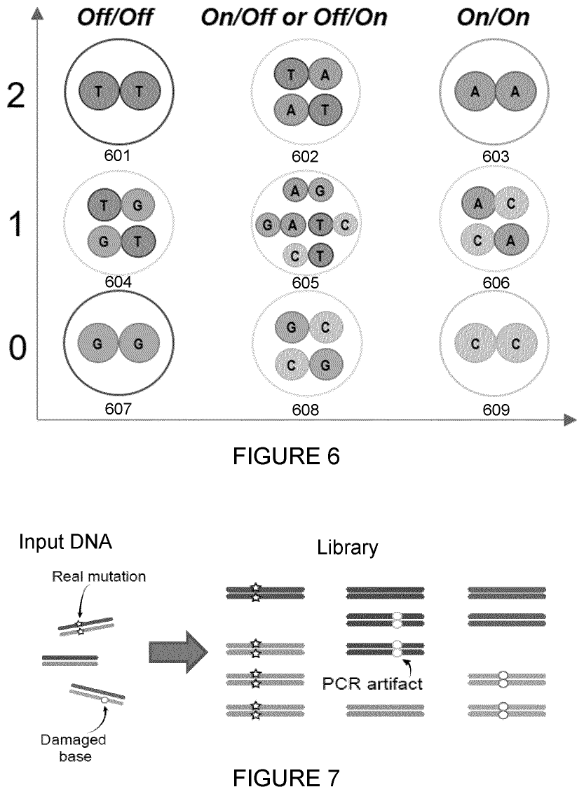

The scatter plot of FIG. 6 shows nine distributions (or bins) of intensity values from the combination of two co-localized signals of substantially equal intensity (e.g. produced by the clusters shown in FIG. 4A or 4B)

The intensity values shown in FIG. 6 may be up to a scale or normalization factor; the units of the intensity values may be arbitrary or relative (i.e., representing the ratio of the actual intensity to a reference intensity). The sum of the signal from the extended first portion primers 402a and the signal from the extended second portion primers 402b results in a combined signal. The combined signal may be captured by the first optical channel and the second optical channel (e.g., the “IMAGE 1” channel and the “IMAGE 2” channel in FIG. 5B). The computer system can map the combined signal from a cluster into one of the nine bins, for example by fitting one or more Gaussian mixture models (GMMs), and thus determine sequence information relating to the added nucleobase at the extended first portion primers 402a and the added nucleobase at the extended second portion primers 402b.

Bins are selected based upon the combined intensity of the signals originating from each sequence portion sensed during the base calling cycle. For example, bin 603 may be selected following the detection of a high-intensity (or “on/on”) signal in the first channel and a high-intensity signal in the second channel. Bin 606 may be selected following the detection of a high-intensity signal in the first channel and an intermediate-intensity (“on/off” of “off/on”) signal in the second channel. Bin 609 may be selected following the detection of a high-intensity signal in the first channel and a low-intensity or zero-intensity (“off/off”) signal in the second channel. Bin 602 may be selected following the detection of an intermediate-intensity signal in the first channel and a high-intensity signal in the second channel. Bin 605 may be selected following the detection of an intermediate-intensity signal in the first channel and an intermediate-intensity signal in the second channel. Bin 808 may be selected following the detection of an intermediate-intensity signal in the first channel and a low-intensity or zero-intensity signal in the second channel. Bin 601 may be selected following the detection of a low-intensity signal in the first channel and a high-intensity signal in the second channel. Bin 604 may be selected following the detection of a low-intensity or zero-intensity signal in the first channel and an intermediate-intensity signal in the second channel. Bin 607 may be selected following the detection of a low-intensity or zero-intensity signal in the first channel and a low-intensity signal in the second channel.

Four of the nine bins represent matches between respective nucleobases of the two polynucleotide sequence portions sensed during the cycle (bins 601,603,607, and 609). In response to mapping the combined signal to a bin representing a match, the computer processor may detect a match between the first and second sequence portions at the sensed position. In response to mapping the combined signal to a bin representing a match, the computer processor may base call the respective nucleobases. For example, when the combined signal is mapped to bin 601 for a base calling cycle, the computer processor base calls both the added nucleobase at the extended first portions primers 402a and the added nucleobase at the extended second portion primers 402b as T. When the combined signal is mapped to bin 603 for the base calling cycle, the processor base calls both the added nucleobase at the extended first portion primers 402a and the added nucleobase at the extended second portion primers 402b as A. When the combined signal is mapped to bin 607 for the base calling cycle, the processor base calls both the added nucleobase at the extended first portion primers 402a and the added nucleobase at the extended second portion primers 402b as G. When the combined signal is mapped to bin 609 for the base calling cycle, the processor base calls both the added nucleobase at the extended first portion primers 402a and the added nucleobase at the extended second portion primers 402b as C.

The remaining five bins are “ambiguous”. That is to say that these bins each represent more than one possible combination of first and second nucleobases. Bins 602, 604, 606, and 608 each represent two possible combinations of first and second nucleobases. Bin 605, meanwhile, represents four possible combinations. Nevertheless, mapping the combined signal to an ambiguous bin may still allow for sequencing information to be determined. For example, bins 602, 604, 605, 606, and 608 represent mismatches between respective nucleobases of the two polynucleotide sequence portions sensed during the cycle. Therefore, in response to mapping the combined signal to a bin representing a mismatch, the computer processor may detect a mismatch between the first and second polynucleotide sequence portions at the sensed position.

The number of classifications which may be selected based upon the combined signal intensities may be predetermined, for example based on the number of sequence portions of interest expected to be present in the nucleic acid cluster. Whilst FIG. 6 shows a set of nine possible classifications, the number of classifications may be greater or smaller.

In addition to identifying matches and mismatches, the mapping of the combined signal to each of the different bins (e.g. in combination with additional knowledge, such as the library preparation methods used) can provide additional information about the first and second sequence portions, or about sequences from which the first and second sequence portions were derived. For example, given the nucleic acid material input and the processing methods used to generate the nucleic acid clusters, the first and second sequence portions may be expected to be identical at a given position. In this case, the mapping of the combined signal to a bin representing a mismatch may be indicative of an error introduced during library preparation. Alternatively, the first and second sequence portions may be expected to be different, for example due to deliberate sequence modifications introduced during library preparation.

### Detecting Library Preparation Errors

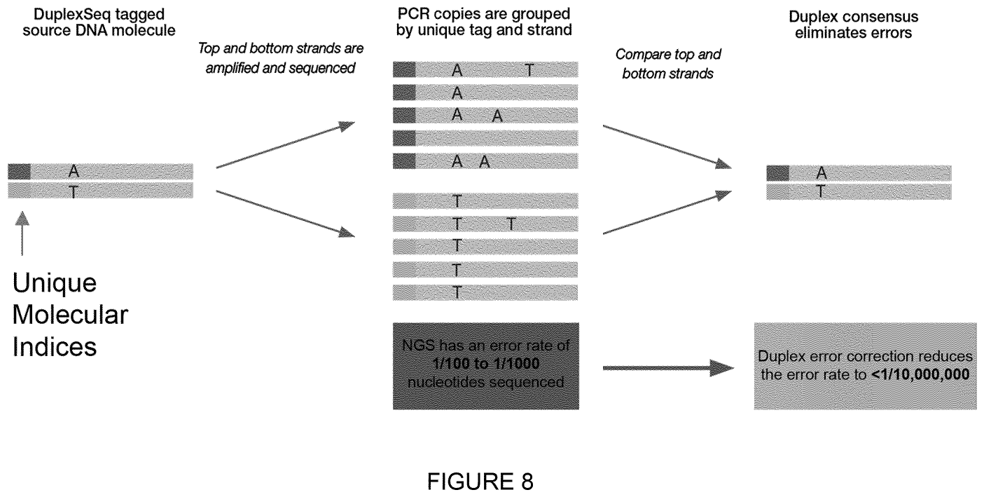

Errors arise during NGS library preparation, for example due to PCR artefacts or DNA damage. The error rate is determined by the library preparation method used, for example the number of cycles of PCR amplification carried out, and a typical error rate may be of the order of 0.1%. This limits the sensitivity of diagnostic assays based on the sequencing method, and may obscure true variants (as shown in FIG. 7). One solution to this problem is to use unique molecular identifiers or indices (UMIs) to distinguish true (e.g. rare) mutations from mutations arising due to library preparation errors (as shown in FIG. 8). The present methods, however, allow for the identification of library preparation errors from fewer sequencing reads.

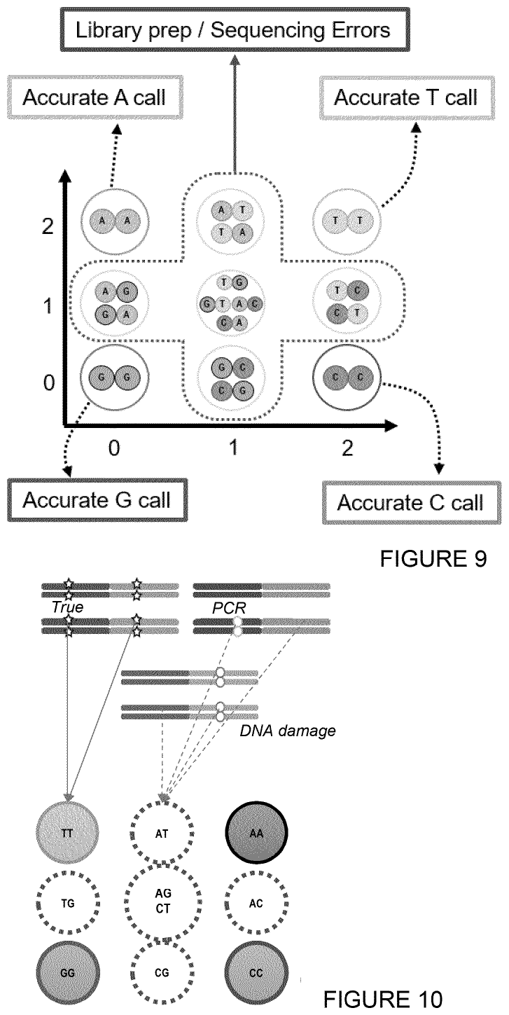

In one example, a plurality of clusters are generated, each comprising at least one first polynucleotide sequence portion and at least one second polynucleotide sequence portion for sensing, wherein the first and second polynucleotide sequence portions will be the same in the absence of any library preparation or sequencing errors. For example, the nucleic acid clusters may be processed such that each of the one or more first polynucleotide sequence portions corresponds to the sequence of a forward strand (or a reverse strand) of a double-stranded target molecule, and the one or more second polynucleotide sequence portions corresponds to a reverse complement strand (or a forward complement strand) of the target. In the absence of any library preparation/sequencing errors, the signals produced by subjecting the two sequence portions to sequencing-by-synthesis will match. The combined signal may therefore be mapped to one of the four “corner” clouds shown in FIG. 9 and FIG. 10, and the identity of the nucleobase at the corresponding position of the original target molecule can be determined. Should the identity of the nucleobase at that position suggest a rare, or even unknown, variant, it can be determined with a high level of confidence that the base call represents a true variant, as opposed to a library preparation error. If, on the other hand, the combined signal is mapped to any of the other clouds, this indicates that the sequences of the first and second polynucleotide sequence portions do not match, and that an error has occurred in library preparation. Therefore, in response to mapping the combined signal to a classification representing a mismatch between the two nucleobases, a library preparation error may be identified.

Depending upon the library preparation methods used, it is not necessarily the case, however, that a match between the first and second nucleobases added to the first and second polynucleotide sequence portions is indicative of the absence of a library preparation error. Similarly, it is not necessarily the case that a mismatch between the first and second nucleobases is indicative of the presence of a library preparation error. For example, one or more sequence modifications may be intentionally introduced during library preparation as described in the example below.

### Detecting Sequence Modifications

The present technology also allows for the detection of sequence modifications (e.g. deliberate sequence modifications) made during library preparation. In particular, a priori information of a modification performed to obtain a sequence may be used to obtain information associated with the modification.

Of the many possible DNA modifications, the methylation of cytosines is the most frequently observed in relation to gene regulation. In order to determine the methylation profile of a nucleic acid sequence, it is known to treat the nucleic acid sequence (e.g. chemically or enzymatically) to convert either methylated or unmethylated cytosine to a different base. For example, bisulfite treatment may be used to convert unmethylated cytosine to uracil. Alternatively, borane treatment, or enzymatic conversion (e.g. using an APOBEC or activation-induced cytidine deaminase (AID) enzyme) may be used to convert 5-methylcytosine to thymine. According to prior methods, inferring methylation status requires conversion, sequencing, and comparison either to a database reference, to an unconverted reference, or to an opposite strand in a consensus pileup. The present methods, however, allow for the methylation status of a sequence to be determined in real-time, from a single sequencing run, and without the need for alignment.

In one example, a double-stranded polynucleotide to be sequenced is treated using a conversion reagent to convert 5-methylcytosine to thymine or cytosine to uracil. Nucleic acid clusters may then be prepared, each comprising one or more first polynucleotide sequence portions corresponding to the sequence of a forward strand (or a reverse strand) of the treated molecule, and one or more second polynucleotide sequence portions corresponding to a reverse complement strand (or a forward complement strand) of the treated molecule. By virtue of the methylation conversion happening prior to the copying process, methylation information of both strands of the original molecule can be determined in a single sequencing run.

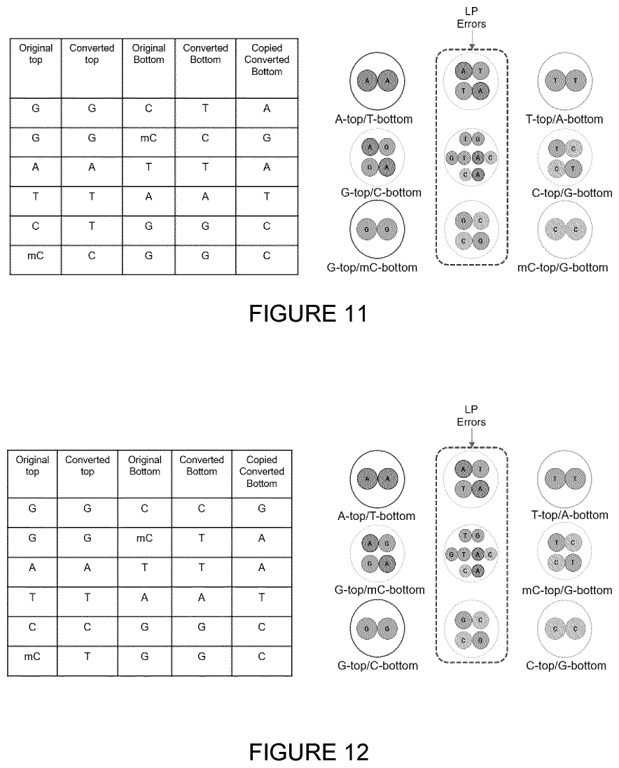

The correspondence between bases in the original target polynucleotide and in the converted strands is shown in FIG. 11, alongside a scatter plot showing the resulting distributions for the combined signal intensities resulting from the simultaneous sequencing of the target sequences. An A-T base pair in the original molecule will result in a match (A/A or TIT) at the corresponding position of the forward and reverse complement strands. An mC-G base pair in the template molecule will also result in a match (GIG or CIC) at the corresponding position of the forward and reverse complement strands. For a C-G base pair, however, the conversion of cytosine to uracil in the forward strand of the target molecule will result in a Tat the corresponding position of the forward strand. Meanwhile, the corresponding position on the reverse complement strand will be occupied by C. Alternatively, the conversion of cytosine to uracil in the reverse strand of the target molecule will result in an A at the corresponding position of the reverse complement strand. Meanwhile, the corresponding position of the forward strand will be occupied by G. Therefore, in response to mapping the combined signal to the distribution representing GIG or C/C, the presence of a methylated base can be determined at the corresponding position in the original target polynucleotide.

FIG. 12 shows the correspondence between bases in the original target polynucleotide and in the converted strands and the resulting distributions for the combined signal intensities using methylcytosine to thymine conversion. An A-T base pair in the template molecule will result in a match (A, A or T, T) at the corresponding position of the forward and reverse complement strands. A C-G base pair in the template molecule will also result in a match (GIG or C/C) at the corresponding position of the forward and reverse complement strands. For a 5mC-G base pair, however, the conversion of 5-methylcytosine to thymine in the “top” strand of the template molecule will result in a T at the corresponding position of the forward strand. Meanwhile, the corresponding position on the reverse complement strand will be occupied by C. Alternatively, the conversion of 5-methylcytosine to thymine in the “bottom” strand of the template molecule will result in an A at the corresponding position of the reverse complement strand. Meanwhile, the corresponding position of the forward strand will be occupied by G. Therefore, in response to mapping the combined signal to the distribution representing an A/G, GIA, TIC, or CIT mismatch, the presence of a methylated base can be determined at the corresponding position in the original polynucleotide.

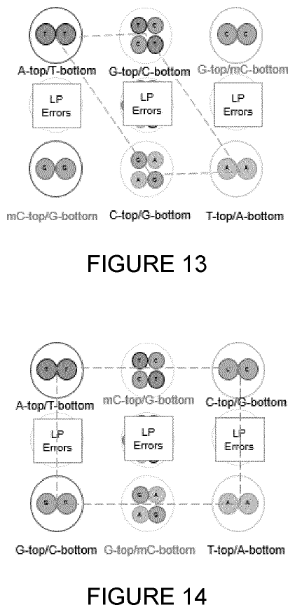

FIG. 13 represents the distributions resulting from the use of an alternative dye-encoding scheme following cytosine to uracil conversion treatment, and FIG. 14 represents the distributions resulting from the use of an alternative dye-encoding scheme following 5-methylcytosine to thymine conversion.

In the present example, for each base pair in the original double-stranded DNA molecule, it may be assumed that there are six possibilities: A-T, T-A, C-G, G-C, mC-G and G-mC. As shown in FIGS. 11-14, each of these possibilities is uniquely represented by one of the plurality of classifications. According to the present methods, it is therefore possible to determine both the sequence and methylation status of a double-stranded DNA target molecule in a single sequencing run.

In addition to determining methylation status, it may also be possible to identify library preparation/sequencing errors. Using the dye-encoding scheme shown in FIG. 11 and FIG. 12, the central column of distributions is indicative of such errors. Using the dye encoding scheme shown in FIG. 13 and FIG. 14, the central row of distributions is indicative of such errors.

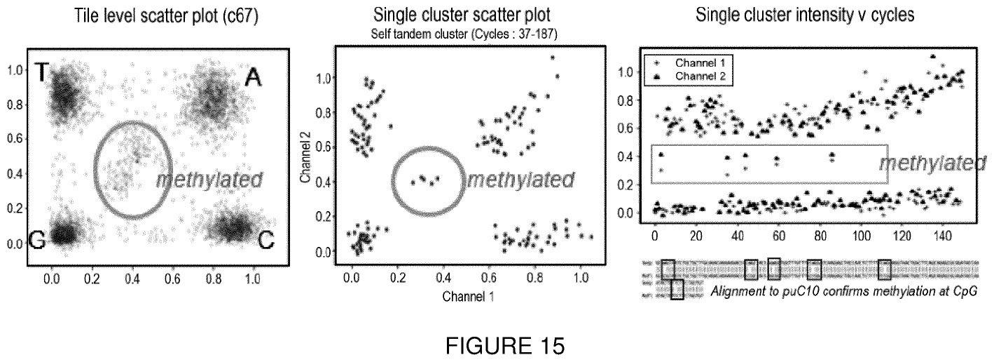

FIG. 15 shows signal distributions resulting from a further dye-encoding scheme. Here, the combined signal may be mapped to one of five distributions (e.g. using a 5 source Gaussian mixture model), with the central distribution corresponding to differences between the two sequence portions by virtue of conversion treatment to identify methylated bases. A further four distributions may also be made available for selection, corresponding to library preparation/sequencing errors.

FIG. 15 (right-hand panel) also illustrates that, where in the absence of a sequence modification the sequences of the first and second polynucleotide sequence portions are expected to be the same, the presence of a sequence modification (e.g. a base conversion) may be determined in response to classifying one or both of the combined intensity of the first and second signals and the combined intensity of the third and fourth signals as being of intermediate intensity (e.g. relative to the combined intensity of the first and second signals and the combined intensity of the third and fourth signals obtained in one or more other base calling cycles of the plurality of base calling cycles).

### Optimisation of the Dye Encoding Scheme

The dye-encoding scheme may be optimised to allow for different combinations of first and second nucleobases to be resolved. This may be particularly useful where sequence modifications of a known type have been introduced into one or more of the first and second sequence portions. For example, where sequence modifications have been introduced that result in the conversion of one nucleobase to another in one of the first and second sequence portions, the dye-encoding scheme may be selected such that the resulting combination of first and second nucleobases do not fall within the central bin (which represents four different nucleobase combinations).

In the case of methylcytosine to thymine conversion, a TIC, or GIA mismatch between the forward and reverse complement strands is indicative of the presence of a 5mC-G base pair at the corresponding position of the target molecule. The dye-encoding scheme may therefore be designed such that these mismatches may be resolved from other possible combinations of nucleobases. This may be achieved by detecting light emissions from A and T bases in a first illumination cycle, and from C and T bases in a second illumination cycle. In another example, light emissions may be detected from C and G bases in a first illumination cycle, and from C and T bases in a second illumination cycle. In another example, light emissions may be detected from C and A bases in a first illumination cycle, and from C and G bases in a second illumination cycle.

In the case of cytosine to uracil conversion, a C/C or GIG match between the forward and reverse complement strands is indicative of the presence of a 5mC-G base pair at the corresponding position of the template molecule. In this case, a 5mC-G base pair will always be resolvable. However, the dye-encoding scheme can still be designed to optimise the resolution between unmodified bases.

### Simplified Sequencing Workflow

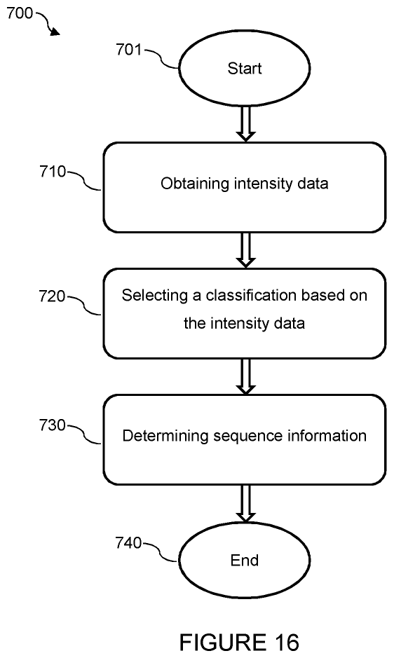

FIG. 16 is a flow diagram showing a method 700 of determining sequence information according to the present disclosure. The described method allows for the determination of sequence information from two or more polynucleotide sequence portions in a single sequencing run from a single combined signal obtained from the two or more portions.

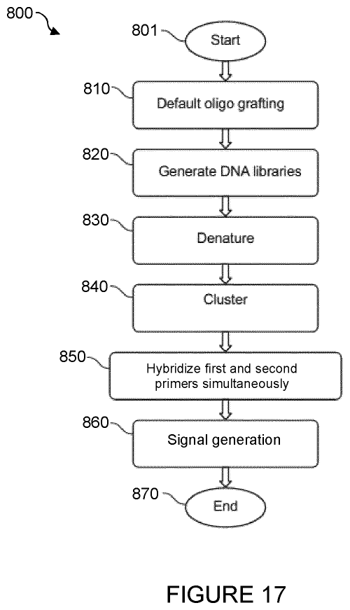

As shown in FIG. 17, the disclosed method 700 may start from block 701. The method may then move to block 710.

At block 710, intensity data is obtained. The intensity data includes first intensity data and second intensity data. The first intensity data comprises a combined intensity of a first signal obtained based upon a respective first nucleobase of at least one first polynucleotide sequence portion and a second signal obtained based upon a respective second nucleobase of at least one second polynucleotide sequence portion. Similarly, the second intensity data comprises a combined intensity of a third signal obtained based upon the respective first nucleobase of the at least one first polynucleotide sequence portion and a fourth signal obtained based upon the respective second nucleobase of the at least one second polynucleotide sequence portion.

As described above, polynucleotide molecules comprising the at least one first polynucleotide sequence portion and the at least one second polynucleotide sequence portion may be arranged on the flow cell such that light emissions from the first and second portions are detected by a single sensing portion and/or may comprise a single cluster such that light emissions from each of the respective two polynucleotide sequence portions cannot be spatially resolved.

In one example, the signals may be generated according to the method shown in FIG. 17.

In one example, obtaining the intensity data comprises selecting intensity data, for example based upon a chastity score. A chastity score may be calculated as the ratio of the brightest base intensity divided by the sum of the brightest and second brightest base intensities. In one example, high-quality data corresponding to two sequence portions with a substantially equal intensity ratio may have a chastity score of around 0.8 to 0.9, for example 0.89-0.9.

After the intensity data has been obtained, the method may proceed to block 720. In this step, one of a plurality of classifications is selected based on the intensity data. Each classification represents one or more possible combinations of respective first and second nucleobases, and at least one classification of the plurality of classifications represents more than one possible combination of respective first and second nucleobases. In one example, the plurality of classifications comprises nine classifications as shown in FIG. 6. Selecting the classification based on the first and second intensity data comprises selecting the classification based on the combined intensity of the first and second signals and the combined intensity of the third and fourth signals, for example using one or more Gaussian mixture model (GMMs).

The method may then proceed to block 730, where sequence information of the respective first and second nucleobases is determined based on the classification selected in block 720. The light emissions generated during a cycle of a sequencing-by-synthesis method are indicative of the identity of the nucleobase(s) added to the sequencing primers undergoing extension. For example, it may be determined that there is a match or a mismatch between the respective first and second nucleobases. Where it is determined that there is a match between the first and second respective nucleobases, the nucleobases may be base called. Whether there is a match or a mismatch, additional or alternative information may be obtained, as described above. It will be appreciated that there is a direct correspondence between the identity of the nucleobases incorporated into the sequencing primers and the identity of the complementary base at the corresponding position of the sequence bound to the flow cell. Therefore, any references herein to the base calling of respective nucleobases of polynucleotide sequence portions encompasses the base calling of nucleobases hybridized to the polynucleotide sequence portions and, alternatively or additionally, the identification of the corresponding nucleobases of the respective portions. The method may then end at block 740.

FIG. 17 is a flow diagram showing a method 800 by which the signals discussed in relation to block 710 of FIG. 16 may be generated. The method may start from block 801.

The method may then move to block 810, default oligo grafting, which may include the attachment of oligonucleotide anchors/graft sequences to a planar, optically transparent surface of the flow cell. The method may then move to block 820, generating DNA libraries from a sample, where template polynucleotides in a sample may be end-repaired to generate 5′-phosphorylated blunt ends, and the polymerase activity of Kienow fragment may be used to add a single A base to the 3′ end of the blunt phosphorylated nucleic acid fragments. This addition prepares the nucleic acid fragments for ligation to oligonucleotide adapters, which have an overhang of a single T base at their 3′ end to increase ligation efficiency. The adapter oligonucleotides are complementary to the flow cell anchor oligos.

After DNA library generation, the method may then move to block 830, denaturing the double stranded DNA libraries to generate single stranded template polynucleotides for seeding on the flow cell. The method may then move to block 840, clustering from the single stranded template polynucleotides. Under limiting-dilution conditions, adapter-modified, single-stranded template polynucleotides are added to the flow cell and immobilized by hybridization to the anchor oligos. Attached nucleic acid fragments are extended and bridge amplified to create an ultra-high density sequencing flow cell with hundreds of millions of clusters, each containing about 1,000 copies of the same template. Details regarding enrichment of nucleic acids using cluster amplification may be found in Kozarewa et al., Nature Methods 6:291-295 (2009), which is incorporated herein by reference.

After cluster generation, the method may directly move to block 850, hybridizing/annealing first and second primers 402a, 402b simultaneously to both the first and second polynucleotide sequence portions 401a, 401b on the flow cell 410. Next, the method may move to block 860 of signal generation. Signal generation proceeds by simultaneously extending the hybridized primers 402a, 402b. With each cycle, fluorescently tagged nucleotides compete for addition to the growing chains of extended primers. Only one is incorporated at a primer location based on the sequence of the template strand. After the addition of nucleotides, the cluster is excited by alight source, and characteristic fluorescent signals are emitted. The emission spectra and the signal intensities uniquely determine the base call. Hundreds of millions of nucleic acid clusters, or thousands to tens of thousands of millions of clusters, may be sequenced in a massively parallel manner. After sequencing the polynucleotide sequence portions 401a, 401b on the flow cell 410, the method may end at block 870.

Sequencing generally comprises four fundamental steps: 1) library preparation to form a plurality of target polynucleotides for identification; 2) cluster generation to form an array of amplified template polynucleotides; 3) sequencing the cluster array of amplified template polynucleotides; and 4) data analysis to identify characteristics of the target polynucleotides from the amplified template polynucleotide sequences. These steps are described in greater detail below. In particular, in one example, the templates to be generated from the libraries may include a concatenated polynucleotide sequence comprising a first portion and a second portion. In another example, the templates to be generated from the libraries may include separate polynucleotide sequences, in particular a first polynucleotide sequence comprising a first portion and a second polynucleotide sequence comprising a second portion.

### Library Strands and Template Terminology

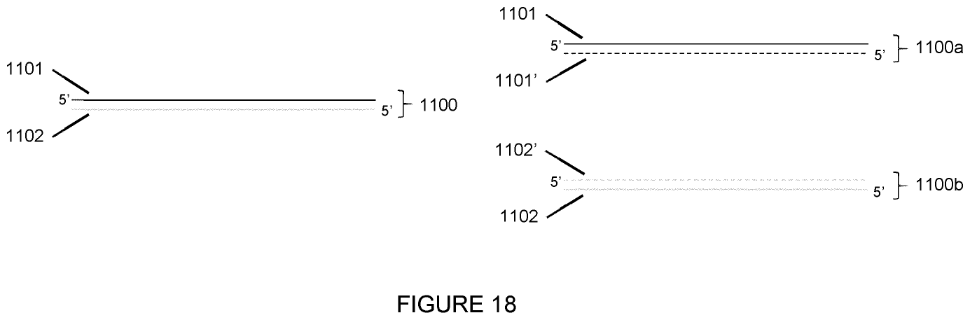

For a given double-stranded polynucleotide sequence 1100 to be identified, the polynucleotide sequence 1100 comprises a forward strand of the sequence 1101 and a reverse strand of the sequence 1102. See FIG. 18.

When the polynucleotide sequence 1100 is replicated (e.g. using a DNA/RNA polymerase), complementary versions of the forward strand 1101 of the sequence 1100 and the reverse strand 1102 of the sequence 1100 are generated. Thus, replication of the polynucleotide sequence 1100 provides a double-stranded polynucleotide sequence 1100a that comprises a forward strand of the sequence 1101 and a forward complement strand of the sequence 1101′, and a double-stranded polynucleotide sequence 1100b that comprises a reverse strand of the sequence 1102 and a reverse complement strand of the sequence 1102′.

The term “template” may be used to describe a complementary version of the double-stranded polynucleotide sequence 1100. As such, the “template” comprises a forward complement strand of the sequence 1101′ and a reverse complement strand of the sequence 1102′. Thus, by using the forward complement strand of the sequence 1101′ as a template for complementary base pairing, a sequencing process (e.g. a sequencing-by-synthesis or a sequencing-by-ligation process) reproduces information that was present in the original forward strand of the sequence 1101. Similarly, by using the reverse complement strand of the sequence 1102′ as a template for complementary base pairing, a sequencing process (e.g. a sequencing-by-synthesis or a sequencing-by-ligation process) reproduces information that was present in the original reverse strand of the sequence 1102.

The two strands in the template may also be referred to as a forward strand of the template 1101′ and a reverse strand of the template 1102′. The complement of the forward strand of the template 1101′ is termed the forward complement strand of the template 1101, whilst the complement of the reverse strand of the template 1102′ is termed the reverse complement strand of the template 1102.

Generally, where forward strand, reverse strand, forward complement strand, and reverse complement strand are used herein without qualifying whether they are with respect to the original polynucleotide sequence 1100 or with respect to the “template”, these terms may be interpreted as referring to the “template”.

| Language for original | Corresponding language for the |
|---|---|
| polynucleotide sequence 1100 | “template” |
| Forward strand of the | Forward complement strand of the |
| sequence 1101 | template 1101 (sometimes referred to |
| | herein as forward complement strand |
| | 1101) |
| Reverse strand of the | Reverse complement strand of the |
| sequence 1102 | template 1102 (sometimes referred to |
| | herein as reverse complement strand |
| | 1102) |
| Forward complement strand | Forward strand of the template 1101′ |
| of the sequence 1101′ | (sometimes referred to herein as forward |
| | strand 1101′) |
| Reverse complement strand | Reverse strand of the template 1102′ |
| of the sequence 1102′ | (sometimes referred to herein as reverse |
| | strand 1102′) |

### Library Preparation

Library preparation is the first step in any high-throughput sequencing platform. These libraries allow templates to be generated via complementary base pairing that can subsequently be clustered and amplified. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). The original sample DNA fragments are referred to as “inserts”. The target polynucleotides may advantageously also be size-fractionated prior to modification with the adaptor sequences.

### Library Preparation (Concatenated Polynucleotide Sequences)

In one example, the templates to be generated from the libraries may include a concatenated polynucleotide sequence comprising a first portion and a second portion. Generating these templates from particular libraries may be performed according to methods known to persons of skill in the art. However, some example approaches of preparing libraries suitable for generation of such templates are described below.

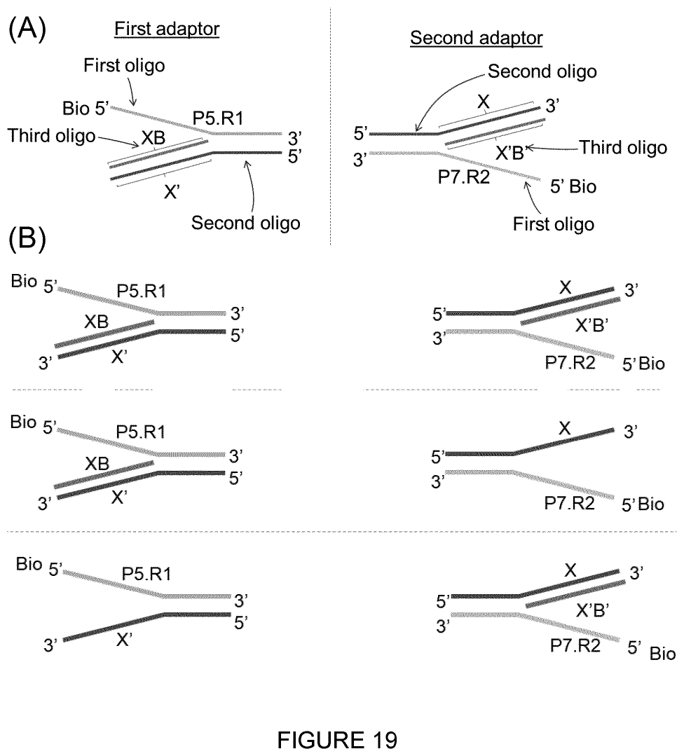

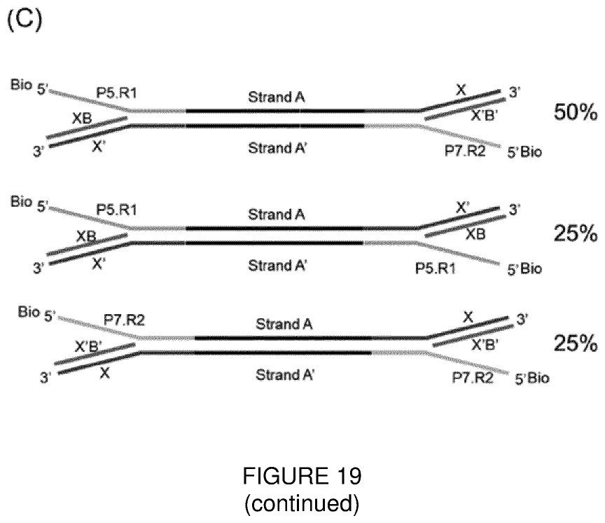

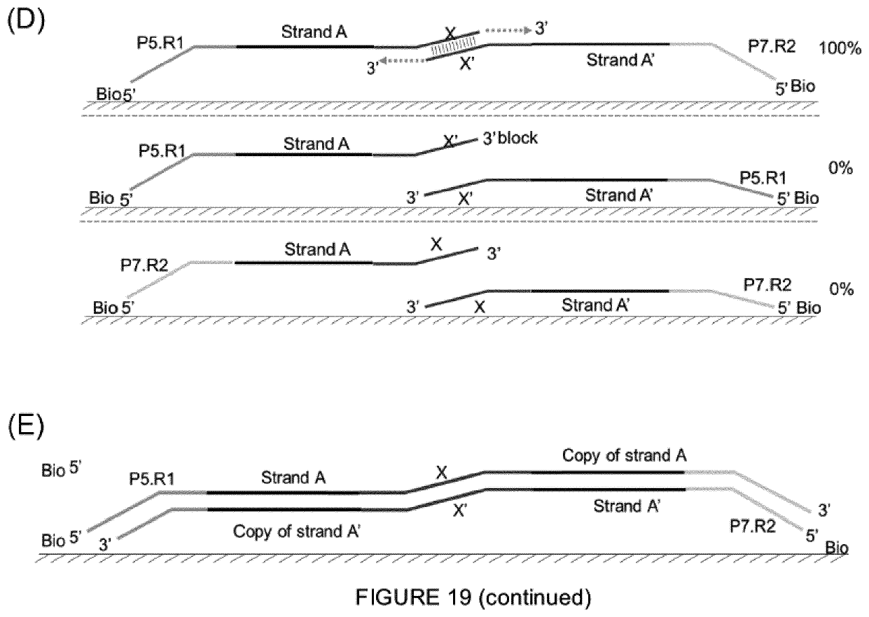

In some embodiments, the library may be prepared by using a tandem insert method described in more detail in e.g. WO 2022/087150, which is incorporated herein by reference. This procedure may be used, for example, for preparing templates comprising concatenated polynucleotide sequences comprising a first portion and a second portion, wherein the first portion is a forward strand of the template, and the second portion is a reverse complement strand of the template (or alternatively, wherein the first portion is a reverse strand of the template, and the second portion is a forward complement strand of the template). Such libraries may also be referred to as cross-tandem inserts. A representative process for conducting a tandem insert method is shown in FIG. 19A to 19E.

The processes described above in relation to tandem insert methods generate libraries that have concatenated polynucleotides.

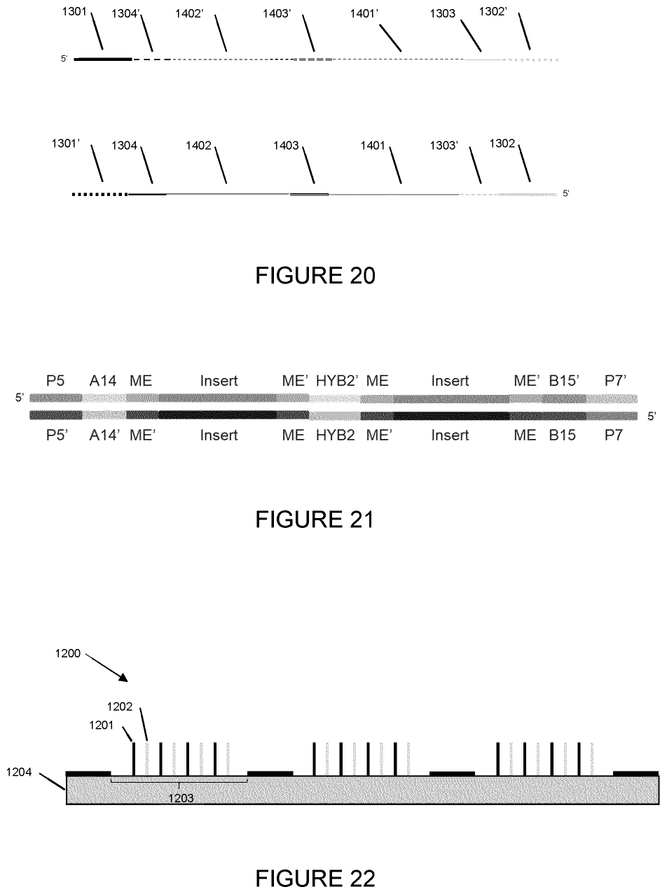

Thus, one strand of a concatenated polynucleotide within a polynucleotide library may comprise, in a 5′ to 3′ direction, a second primer-binding complement sequence 1302 (e.g. P7), a first terminal sequencing primer binding site complement 1303′ (e.g. B15-ME; or if ME is not present, then B15), a first insert sequence 1401, a hybridisation complement sequence 1403 (e.g. ME′-HYB2-ME; or if ME′ and ME are not present, then HYB2), a second insert sequence 1402, a second terminal sequencing primer binding site 1304 (e.g. ME′-A14′; or if ME′ is not present, then A14′), and a first primer-binding sequence 1301′ (e.g. PS′) (FIGS. 20 and 21—bottom strand).

Although not shown in FIGS. 20 and 21, the strand may further comprise one or more index sequences. As such, a first index sequence (e.g. i7) may be provided between the second primer-binding complement sequence 1302 (e.g. P7) and the first terminal sequencing primer binding site complement 1303′ (e.g. B15-ME; or if ME is not present, then B15). Separately, or in addition, a second index complement sequence (e.g. i5′) may be provided between the second terminal sequencing primer binding site 1304 (e.g. ME′-A14′) and the first primer-binding sequence 1301′ (e.g. PS′). Thus, in some embodiments, one strand of a polynucleotide within a polynucleotide library may comprise, in a 5′ to 3′ direction, a second primer-binding complement sequence 1302 (e.g. P7), a first index sequence (e.g. i7), a first terminal sequencing primer binding site complement 1303′ (e.g. B15-ME; or if ME is not present, then B15), a first insert sequence 1401, a hybridisation complement sequence 1403 (e.g. ME′-HYB2-ME; or if ME′ and ME are not present, then HYB2), a second insert sequence 1402, a second terminal sequencing primer binding site 1304 (e.g. ME′-A14′; or if ME′ is not present, then A14′), a second index complement sequence (e.g. i5′), and a first primer-binding sequence 1301′ (e.g. PS′)

Another strand of a concatenated polynucleotide within a polynucleotide library may comprise, in a 5′ to 3′ direction, a first primer-binding complement sequence 1301 (e.g. PS), a second terminal sequencing primer binding site complement 1304′ (e.g. A14-ME; or if ME is not present, then A14), a second insert complement sequence 1402′, a hybridisation sequence 1403′ (e.g. ME′-HYB2′-ME; or if ME′ and ME are not present, then HYB2′), a first insert complement sequence 1401′, a first terminal sequencing primer binding site 1303 (e.g. ME′-B15′; or if ME′ is not present, then B15′), and a second primer-binding sequence 1302′ (e.g. P7′) (FIGS. 20 and 21—top strand).

Although not shown in FIGS. 20 and 21, the another strand may further comprise one or more index sequences. As such, a second index sequence (e.g. i5) may be provided between the first primer-binding complement sequence 1301 (e.g. PS) and the second terminal sequencing primer binding site complement 1304′ (e.g. A14-ME; or if ME is not present, then A14). Separately, or in addition, a first index complement sequence (e.g. i7′) may be provided between the first terminal sequencing primer binding site 1303 (e.g. ME′-B15′; or if ME′ is not present, then B15′) and the second primer-binding sequence 1302′ (e.g. P7′). Thus, in some embodiments, another strand of a polynucleotide within a polynucleotide library may comprise, in a 5′ to 3′ direction, a first primer-binding complement sequence 1301 (e.g. PS), a second index sequence (e.g. i5), a second terminal sequencing primer binding site complement 1304′ (e.g. A14-ME; or if ME is not present, then A14).), a second insert complement sequence 1402′, a hybridisation sequence 1403′ (e.g. ME′-HYB2′-ME; or if ME′ and ME are not present, then HYB2′), a first insert complement sequence 1401′, a first terminal sequencing primer binding site 1303 (e.g. ME′-B15′; or if ME′ is not present, then B15′), a first index complement sequence (e.g. i7), and a second primer-binding sequence 1302′ (e.g. PT).

As described herein, the first insert sequence 1401 and the second insert sequence 1402 may comprise different types of library sequences.

In one embodiment, the first insert sequence 1401 may comprise a forward strand of the sequence 1101, and the second insert sequence may comprise a reverse complement strand of the sequence 1102′ (or the first insert sequence 1401 may comprise a reverse strand of the sequence 1102, and the second insert sequence 1402 may comprise a forward complement strand of the sequence 1101′), for example where the library is prepared using a tandem insert method.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (such as a primer-binding sequence and a sequencing primer binding site, or a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

As used herein, an “adaptor” refers to a sequence that comprises a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the PS' and P7′ primer-binding sequences are complementary to short primer sequences (or lawn primers) present on the surface of a flow cell. Binding of PS' and P7′ to their complements (PS and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

In concatenated strands, the hybridisation sequence (or the hybridisation sequence complement) may comprise an internal sequencing primer binding site. In other words, an internal sequencing primer binding site may form part of the hybridisation sequence. For example, ME′-HYB2 (or ME′-HYB2) may act as an internal sequencing primer binding site to which a sequencing primer can bind. Alternatively, the hybridisation sequence may be an internal sequencing primer binding site. For example, HYB2 (or HYB2′) may act as an internal sequencing primer binding site to which a sequencing primer can bind. Accordingly, we may refer to the hybridisation site herein as comprising a second sequencing primer binding site, or as a second sequencing primer binding site.

The target polynucleotide (or in some embodiments, the polynucleotide library) may be pre-treated to allow sequencing of modified cytosines. Such methods are described in further detail herein.

### Cluster Generation and Amplification (Concatenated Polynucleotide Sequences)

Once a double stranded nucleic acid library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning. A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example PS and P7 lawn primers).

Thus, embodiments of the present invention may be performed on a solid support 1200, such as a flowcell. However, in alternative embodiments, seeding and clustering can be conducted off-flowcell using other types of solid support.

The solid support 1200 may comprise a substrate 1204. See FIG. 22. The substrate 1204 comprises at least one well 1203 (e.g. a nanowell), and typically comprises a plurality of wells 1203 (e.g. a plurality of nanowells).

The solid support may comprise at least one first immobilised primer and at least one second immobilised primer.

Thus, each well 1203 may comprise at least one first immobilised primer 1201, and typically may comprise a plurality of first immobilised primers 1201. In addition, each well 1203 may comprise at least one second immobilised primer 1202, and typically may comprise a plurality of second immobilised primers 1202. Thus, each well 1203 may comprise at least one first immobilised primer 1201 and at least one second immobilised primer 1202, and typically may comprise a plurality of first immobilised primers 1201 and a plurality of second immobilised primers 1202.

The first immobilised primer 1201 may be attached via a 5′-end of its polynucleotide chain to the solid support 1200. When extension occurs from first immobilised primer 1201, the extension may be in a direction away from the solid support 1200.

The second immobilised primer 1202 may be attached via a 5′-end of its polynucleotide chain to the solid support 1200. When extension occurs from second immobilised primer 1202, the extension may be in a direction away from the solid support 1200.

The first immobilised primer 1201 may be different to the second immobilised primer 1202 and/or a complement of the second immobilised primer 1202. The second immobilised primer 1202 may be different to the first immobilised primer 1201 and/or a complement of the first immobilised primer 1201.

The (or each of the) first immobilised primer(s) 1201 may comprise a sequence as defined in SEQ ID NO. 1 or 5, or a variant or fragment thereof. The second immobilised primer(s) 1202 may comprise a sequence as defined in SEQ ID NO. 2, or a variant or fragment thereof.

By way of brief example, following attachment of the PS and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, SxSSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either PS' or P7′) which is capable of bridging to the second primer molecule immobilised on the solid support and binding. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products. This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

In some cases, to facilitate sequencing, one set of strands (either the original template strands or the complement strands thereof) may be removed from the solid support leaving either the original template strands or the complement strands. Suitable methods for removing such strands are described in more detail in application number WO 07/010251, the contents of which are incorporated herein by reference in their entirety.

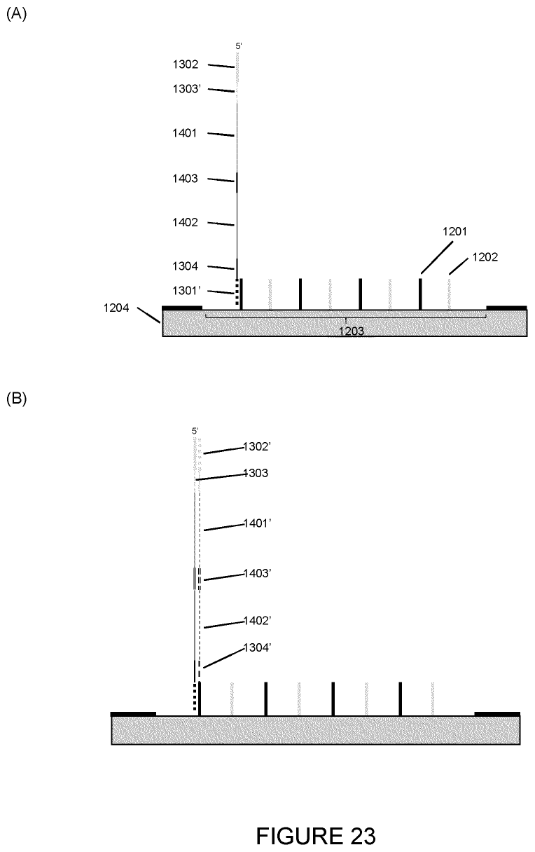

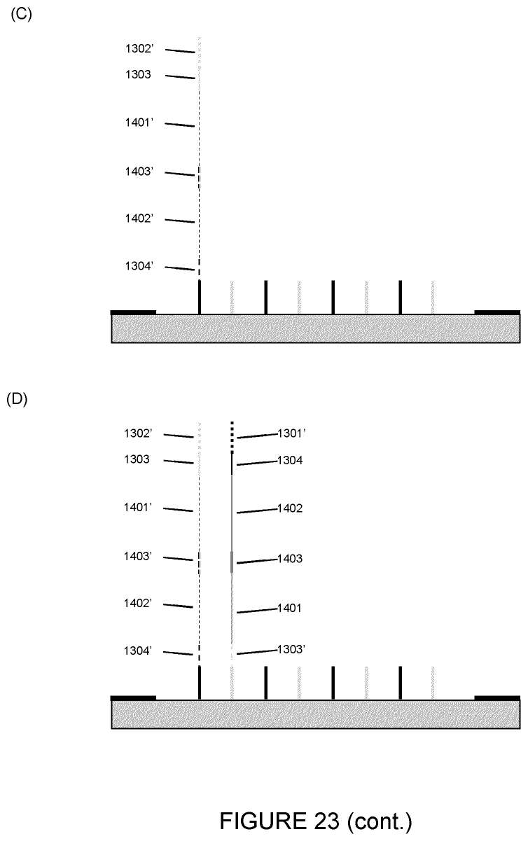

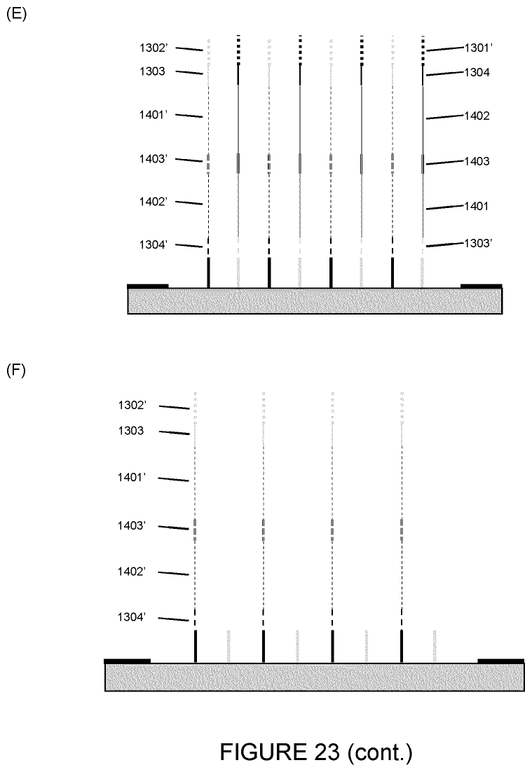

The steps of cluster generation and amplification for templates including a concatenated polynucleotide sequence comprising a first portion and a second portion are illustrated below and in FIG. 23.

In cases where single (concatenated) polynucleotide strands are used, each polynucleotide sequence may be attached (via the 5′-end of the (concatenated) polynucleotide sequence) to a first immobilised primer. Each polynucleotide sequence may comprise a second adaptor sequence, wherein the second adaptor comprises a portion which is substantially complementary to the second immobilised primer (or is substantially complementary to the second immobilised primer). The second adaptor sequence may be at a 3′-end of the (concatenated) polynucleotide sequence.

In an embodiment, a solution comprising a polynucleotide library prepared by a tandem insert method as described above may be flowed across a flowcell.

A particular concatenated polynucleotide strand from the polynucleotide library to be sequenced comprising, in a 5′ to 3′ direction, a second primer-binding complement sequence 1302 (e.g. P7), a first terminal sequencing primer binding site complement 1303′ (e.g. B15-ME), a first insert sequence 1401, a hybridisation complement sequence 1403 (e.g. ME′-HYB2-ME), a second insert sequence 1402, a second terminal sequencing primer binding site 1304 (e.g. ME′-A14′), and a first primer-binding sequence 1301′ (e.g. PS′), may anneal (via the first primer-binding sequence 1301′) to the first immobilised primer 1201 (e.g. PS lawn primer) located within a particular well 1203 (FIG. 23A).

The polynucleotide library may comprise other concatenated polynucleotide strands with different first insert sequences 1401 and second insert sequences 1402. Such other polynucleotide strands may anneal to corresponding first immobilised primers 1201 (e.g. PS lawn primers) in different wells 1203, thus enabling parallel processing of the various different concatenated strands within the polynucleotide library.

A new polynucleotide strand may then be synthesised, extending from the first immobilised primer 1201 (e.g. PS lawn primer) in a direction away from the substrate 1204. By using complementary base-pairing, this generates a template strand comprising, in a 5′ to 3′ direction, the first immobilised primer 1201 (e.g. PS lawn primer) which is attached to the solid support 1200, a second terminal sequencing primer binding site complement 1304′ (e.g. A14-ME; or if ME is not present, then A14), a second insert complement sequence 1402′ (which represents a type of “second portion”), a hybridisation sequence 1403′ (which comprises a type of “second sequencing primer binding site”) (e.g. ME′-HYB2′-ME; or if ME′ and ME are not present, then HYB2′), a first insert complement sequence 1401′ (which represents a type of “first portion”), a first terminal sequencing primer binding site 1303 (which represents a type of “first sequencing primer binding site”) (e.g. ME′-B15′; or if ME′ is not present, then 615′), and a second primer-binding sequence 1302′ (e.g. P7′) (FIG. 23B). Such a process may utilise a polymerase, such as a DNA or RNA polymerase.

If the polynucleotides in the library comprise index sequences, then corresponding index sequences are also produced in the template.

The concatenated polynucleotide strand from the polynucleotide library may then be dehybridised and washed away, leaving a template strand attached to the first immobilised primer 1201 (e.g. PS lawn primer) (FIG. 23C).

The second primer-binding sequence 1302′ (e.g. P7′) on the template strand may then anneal to a second immobilised primer 1202 (e.g. P7 lawn primer) located within the well 1203. This forms a “bridge”.

A new polynucleotide strand may then be synthesised by bridge amplification, extending from the second immobilised primer 1202 (e.g. P7 lawn primer) (initially) in a direction away from the substrate 1204. By using complementary base-pairing, this generates a template strand comprising, in a 5′ to 3′ direction, the second immobilised primer 1202 (e.g. P7 lawn primer) which is attached to the solid support 1200, a first terminal sequencing primer binding site complement 1303′ (e.g. B15-ME; or if ME is not present, then B15), a first insert sequence 1401, a hybridisation complement sequence 1403 (e.g. ME′-HYB2-ME; or if ME′ and ME are not present, then HYB2), a second insert sequence 1402, a second terminal sequencing primer binding site 1304 (e.g. ME′-A14′; or if ME′ is not present, then A14′), and a first primer-binding sequence 1301′ (e.g. PS′). Again, such a process may utilise a polymerase, such as a DNA or RNA polymerase.

The strand attached to the second immobilised primer 1202 (e.g. P7 lawn primer) may then be dehybridised from the strand attached to the first immobilised primer 1201 (e.g. PS lawn primer) (FIG. 23D).

A subsequent bridge amplification cycle can then lead to amplification of the strand attached to the first immobilised primer 1201 (e.g. PS lawn primer) and the strand attached to the second immobilised primer 1202 (e.g. P7 lawn primer). The second primer-binding sequence 1302′ (e.g. P7′) on the template strand attached to the first immobilised primer 1201 (e.g. PS lawn primer) may then anneal to another second immobilised primer 1202 (e.g. P7 lawn primer) located within the well 1203. In a similar fashion, the first primer-binding sequence 1301′ (e.g. PS′) on the template strand attached to the second immobilised primer 1202 (e.g. P7 lawn primer) may then anneal to another first immobilised primer 1201 (e.g. PS lawn primer) located within the well 1203.

Completion of bridge amplification and dehybridisation may then provide an amplified cluster, thus providing a plurality of concatenated polynucleotide sequences comprising a first insert complement sequence 1401′ (i.e. “first portions”) and a second insert complement sequence 1402′ (i.e. second portions”), as well as a plurality of concatenated polynucleotide sequences comprising a first insert sequence 1401 and a second insert sequence 1402 (FIG. 23E).

If desired, further bridge amplification cycles may be conducted to increase the number of polynucleotide sequences within the well 1203.

Before sequencing, one group of strands (either the group of template polynucleotides, or the group of template complement polynucleotides thereof) may be removed from the solid support to form a (monoclonal) cluster, leaving either the templates or the template complements (FIG. 23F).

### Sequencing (Concatenated Polynucleotide Sequences)

In one embodiment, the sequencing process comprises a first sequencing read and second sequencing read. The first sequencing read and the second sequencing read may be conducted concurrently. In other words, the first sequencing read and the second sequencing read may be conducted at the same time.

The first sequencing read may comprise the binding of a first sequencing primer (also known as a read 1 sequencing primer) to the first sequencing primer binding site (e.g. first terminal sequencing primer binding site 1303 in templates including a concatenated polynucleotide sequence comprising a first portion and a second portion). The second sequencing read may comprise the binding of a second sequencing primer (also known as a read 2 sequencing primer) to the second sequencing primer binding site (e.g. a portion of hybridisation sequence 1403′ in templates including a concatenated polynucleotide sequence comprising a first portion and a second portion).

This leads to sequencing of the first portion (e.g. first insert complement sequence 1401′ in templates including a concatenated polynucleotide sequence comprising a first portion and a second portion) and the second portion (e.g. second insert complement sequence 1402′ in templates including a concatenated polynucleotide sequence comprising a first portion and a second portion).

Alternative methods of sequencing include sequencing by ligation, for example as described in U.S. Pat. No. 6,306,597 or WO 06/084132, the contents of which are incorporated herein by reference.

### Sequencing of Modified Cytosines (Concatenated Polynucleotide Sequences)

In one example, at least one polynucleotide sequence may be prepared for detection of modified cytosines, by a method comprising:
synthesising at least one polynucleotide sequence comprising a first portion and a second portion,
wherein the at least one polynucleotide sequence comprises portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template,
wherein the template is generated from a (double-stranded) target polynucleotide to be sequenced via complementary base pairing, and wherein the target polynucleotide has been pre-treated using a conversion reagent,
wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil.

As described herein, the at least one polynucleotide sequence may comprise portions of a double-stranded nucleic acid template, and the first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). Alternatively, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand).

The first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced.

The template is generated from a (double-stranded) target polynucleotide to be sequenced via complementary base pairing. The (double-stranded) target polynucleotide may be one (double-stranded) polynucleotide present in a polynucleotide library to be sequenced. As such, the template allows sequence information to be obtained for that particular polynucleotide.

The method may further comprise a step of preparing the first portion and the second portion for concurrent sequencing.

For example, the method may comprise simultaneously contacting first sequencing primer binding sites located after a 3′-end of the first portions with first primers and second sequencing primer binding sites located after a 3′-end of the second portions with second primers. Thus, the first portions and second portions are primed for concurrent sequencing.

In some embodiments, a proportion of first portions may be capable of generating a first signal and a proportion of second portions may be capable of generating a second signal, wherein an intensity of the first signal is substantially the same as an intensity of the second signal.

The first signal and the second signal may be spatially unresolved (e.g. generated from the same region or substantially overlapping regions).

The first portion may be referred to herein as read 1 (R1). The second portion may be referred to herein as read 2 (R2).

The single (concatenated) polynucleotide strand may be attached to a solid support. This solid support may be a flow cell. The polynucleotide strand may be attached to the solid support in a single well of the solid support.

The polynucleotide strand or strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a single polynucleotide strand with a first and second portion, before sequencing one group of strands (either the group of template polynucleotides, or the group of template complement polynucleotides thereof) may be removed from the solid support, leaving either the templates or the template complements, as explained above. Such a cluster may be considered to be a “monoclonal” cluster.

By “monoclonal” cluster is meant that the population of polynucleotide sequences that are then sequenced (as the next step) are substantially the same—i.e. copies of the same sequence. As such, a “monoclonal” cluster may refer to the population of single polynucleotide molecules within a well that are then sequenced. A “monoclonal” cluster may contain a sufficient number of copies of a single template polynucleotide (or copies of a single template complement polynucleotide) such that the cluster is able to output a signal (e.g. a light signal) that allows sequencing reads to be performed on the “monoclonal” cluster. A “monoclonal” cluster may comprise, for example, about 500 to about 2000 copies, about 600 to about 1800 copies, about 700 to about 1600 copies, about 800 to 1400 copies, about 900 to 1200 copies, or about 1000 copies of a single template polynucleotide (or copies of a single template complement polynucleotide). The copies of the single template polynucleotide (and/or single template complement polynucleotides) may comprise at least about 50%, at least about 80%, at least about 70%, at least about 80%, at least about 90%, or about 95%, 98%, 99% or 100% of all polynucleotides within a single well of the flow cell, and thus providing a substantially monoclonal “cluster”.

The at least one polynucleotide sequence comprising a first portion and a second portion may be prepared using a tandem insert method as described herein. Accordingly, in one embodiment, the step of synthesising the at least one polynucleotide sequence comprising a first portion and a second portion may comprise:
synthesising a first precursor polynucleotide fragment comprising a complement of the first portion and a hybridisation complement sequence,
synthesising a second precursor polynucleotide fragment comprising a second portion and a hybridisation sequence,
annealing the hybridisation complement sequence of the first precursor polynucleotide fragment with the hybridisation sequence on the second precursor polynucleotide fragment to form a hybridised adduct,
synthesising a first precursor polynucleotide sequence by extending the first precursor polynucleotide fragment to form a complement of the second portion, and
synthesising the at least one polynucleotide sequence by forming a complement of the first precursor polynucleotide sequence.

The first precursor polynucleotide fragment may comprise a first sequencing primer binding site complement.

The first sequencing primer binding site complement may be located before a 5′-end of the complement of the first portion, for example immediately before the 5′-end of the complement of the first portion.

The first precursor polynucleotide fragment may comprise a second adaptor complement sequence.

The second adaptor complement sequence may be located before a 5′-end of the complement of the first portion.

The first precursor polynucleotide fragment may comprise a first sequencing primer binding site complement and a second adaptor complement sequence.

The first sequencing primer binding site complement may be located before a 5′-end of the complement of the first portion, and wherein the second adaptor complement sequence may be located before a 5′-end of the first sequencing primer binding site complement.

The first precursor polynucleotide fragment may comprise a second sequencing primer binding site complement.

The hybridisation sequence complement may comprise the second sequencing primer binding site complement.

The second precursor polynucleotide fragment may comprise a first adaptor complement sequence.

In some embodiments, the method may further comprise a step of concurrently sequencing nucleobases in the first portion and the second portion.

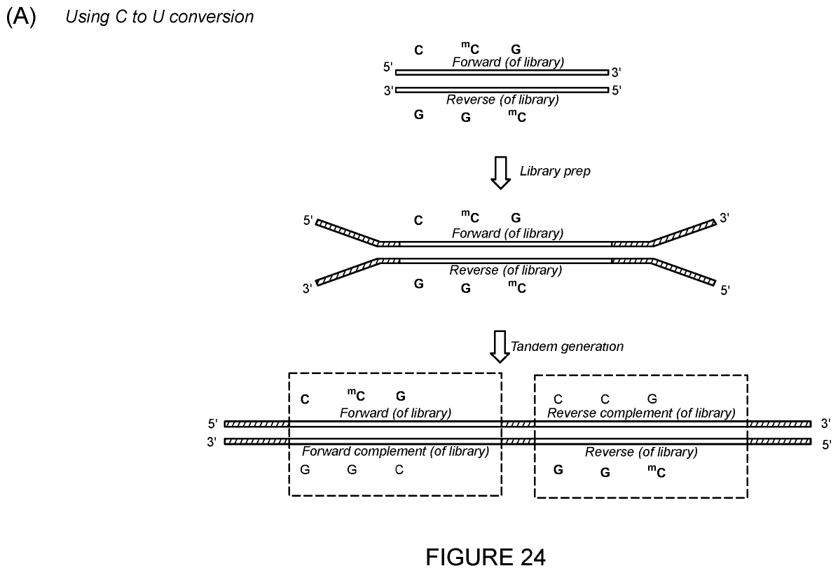

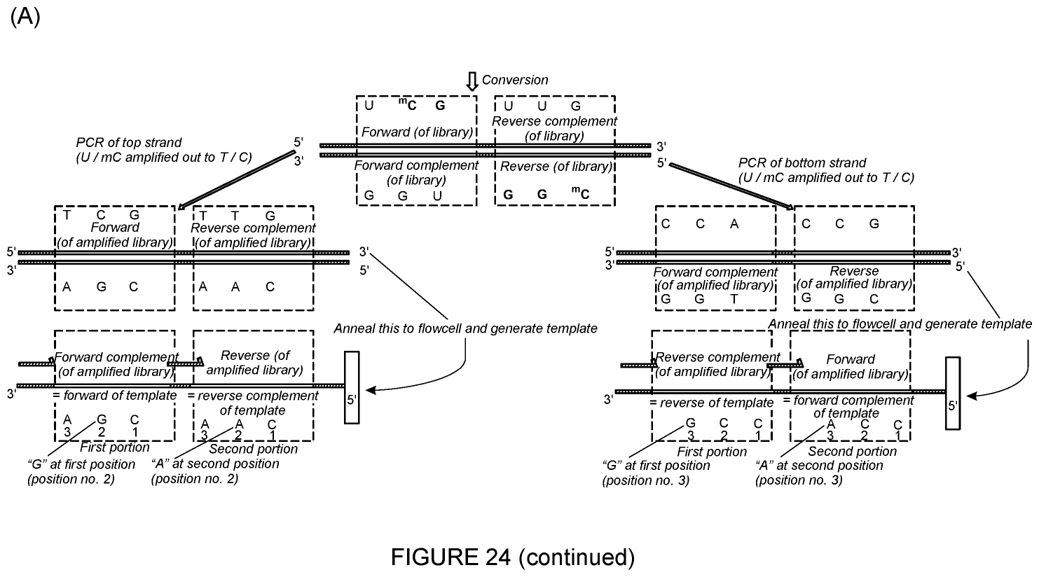

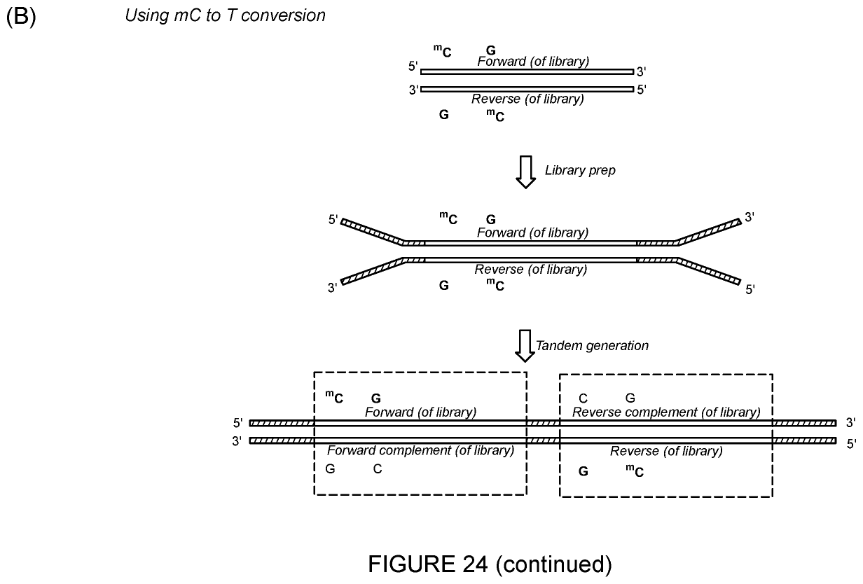

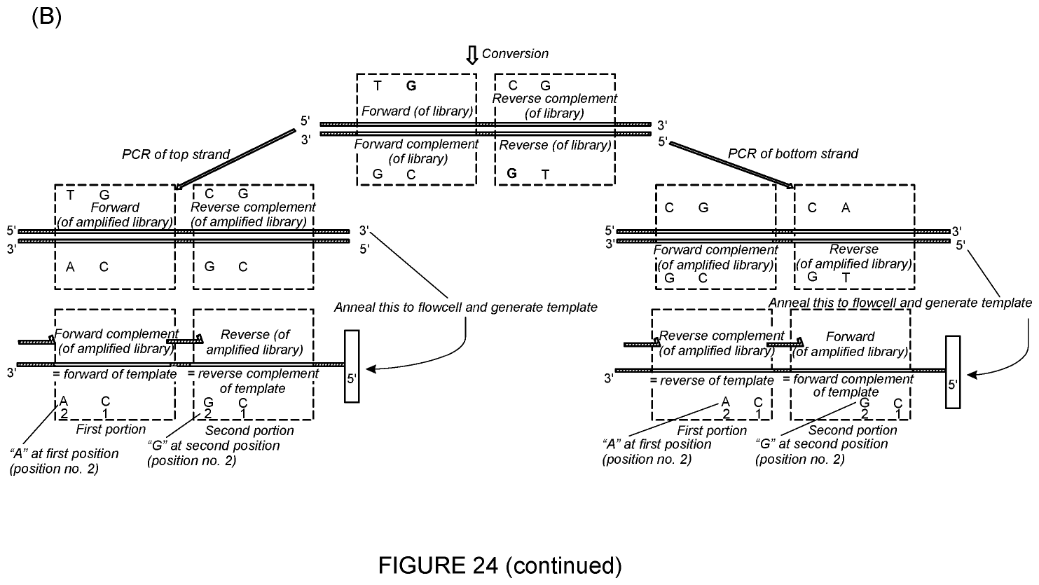

The target polynucleotide (or in some embodiments, the polynucleotide library) may have been pre-treated using a conversion reagent. In some embodiments, the method of preparing at least one polynucleotide sequence for detection of modified cytosines may include a step of treating the target polynucleotide using a conversion agent. FIG. 24 shows the effect of the pre-treatment of the target polynucleotide of various conversion agents on the bases in the resulting template strands.

### Library Preparation (Separate Polynucleotide Sequences)

In another example, the templates to be generated from the libraries may include separate polynucleotide sequences, in particular a first polynucleotide sequence comprising a first portion and a second polynucleotide sequence comprising a second portion. Generating these templates from particular libraries may be performed according to methods known to persons of skill in the art. However, some example approaches of preparing libraries suitable for generation of such templates are described below.

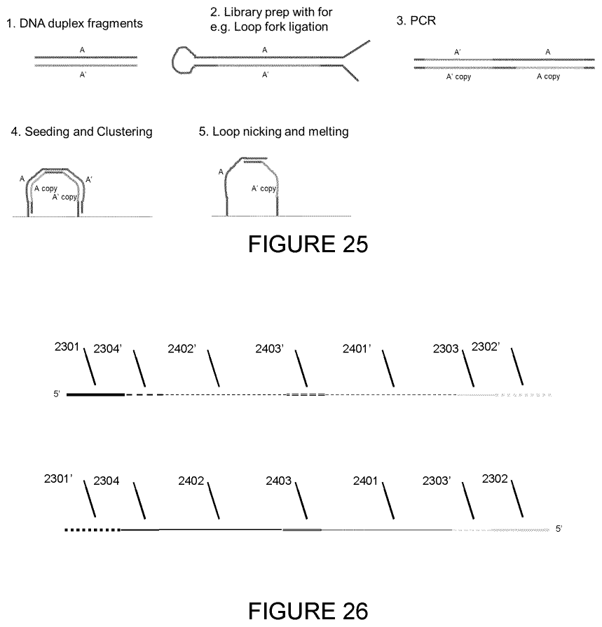

In some embodiments, the library may be prepared using a loop fork method, which is described below. This procedure may be used, for example, for preparing templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template). A representative process for conducting a loop fork method is shown in FIG. 25.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be described later, during clustering and amplification, further processes may be used to generate templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template).

The processes described above in relation to loop fork methods generate libraries that have self-tandem insert polynucleotides.

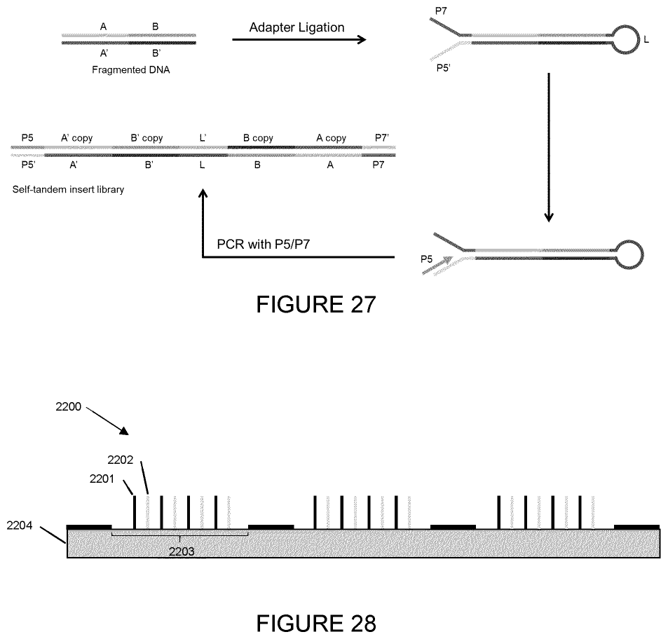

Thus, one strand of a polynucleotide within a polynucleotide library may comprise, in a 5′ to 3′ direction, a second primer-binding complement sequence 2302 (e.g. P7), an optional first terminal sequencing primer binding site complement 2303′, a first insert sequence 2401 (A and B), a loop sequence 2403 (L), a second insert sequence 2402 (B′ and A′), an optional second terminal sequencing primer binding site 2304, and a first primer-binding sequence 2301′ (e.g. PS) (FIGS. 26 and 27—bottom strand).

Alternatively, or in addition, one or more sequencing primer binding sites (or complements) may be provided within the loop sequence 2403 (L).

Although not shown in FIGS. 26 and 27, the strand may further comprise one or more index sequences. As such, a first index sequence (e.g. U7) may be provided between the second primer-binding complement sequence 2302 (e.g. P7) and the optional first terminal sequencing primer binding site complement 2303′. Separately, or in addition, a second index complement sequence (e.g. i5′) may be provided between the optional second terminal sequencing primer binding site 2304 and the first primer-binding sequence 2301′ (e.g. PS). Thus, in some embodiments, one strand of a polynucleotide within a polynucleotide library may comprise, in a 5′ to 3′ direction, a second primer-binding complement sequence 2302 (e.g. P7), a first index sequence (e.g. i7), an optional first terminal sequencing primer binding site complement 2303′, a first insert sequence 2401 (A and B), a loop sequence 2403 (L), a second insert sequence 2402 (B′ and A′), an optional second terminal sequencing primer binding site 2304, a second index complement sequence (e.g. i5′), and a first primer-binding sequence 2301′ (e.g. PS).

Alternatively, or in addition, one or more index sequences (or complements) may be provided within the loop sequence 2403 (L).

Another strand of a polynucleotide within a polynucleotide library may comprise, in a 5′ to 3′ direction, a first primer-binding complement sequence 2301 (e.g. PS), an optional second terminal sequencing primer binding site complement 2304′, a second insert complement sequence 2402′ (A′ copy and B′ copy), a loop complement sequence 2403′ (L′), a first insert complement sequence 2401′ (B copy and A copy), an optional first terminal sequencing primer binding site 2303, and a second primer-binding sequence 2302′ (e.g. P7) (FIGS. 26 and 27—top strand).

Alternatively, or in addition, one or more sequencing primer binding sites (or complements) may be provided within the loop complement sequence 2403′ (L).

Although not shown in FIGS. 26 and 27, the another strand may further comprise one or more index sequences. As such, a second index sequence (e.g. i5) may be provided between the first primer-binding complement sequence 2301 (e.g. PS) and the optional second terminal sequencing primer binding site complement 2304′. Separately, or in addition, a first index complement sequence (e.g. i7′) may be provided between the optional first terminal sequencing primer binding site 2303 and the second primer-binding sequence 2302′ (e.g. P7′). Thus, in some embodiments, another strand of a polynucleotide within a polynucleotide library may comprise, in a 5′ to 3′ direction, a first primer-binding complement sequence 2301 (e.g. PS), a second index sequence (e.g. i5), an optional second terminal sequencing primer binding site complement 2304′, a second insert complement sequence 2402′ (A′ copy and B′ copy), a loop complement sequence 2403′ (L′), a first insert complement sequence 2401′ (B copy and A copy), an optional first terminal sequencing primer binding site 2303, a first index complement sequence (e.g. i7′), and a second primer-binding sequence 2302′ (e.g. P7′).

Alternatively, or in addition, one or more index sequences (or complements) may be provided within the loop complement sequence 2403′ (L′).

In one embodiment, the first insert sequence 2401 may comprise a forward strand of the sequence 2101, and the second insert complement sequence 2402′ may comprise a reverse complement strand of the sequence 2102′ (or the first insert sequence 2401 may comprise a reverse strand of the sequence 2102, and the second insert complement sequence 2402′ may comprise a forward complement strand of the sequence 2101′), for example where the library is prepared using a loop fork method.

Although FIG. 26 shows the presence of a first terminal sequencing primer binding site complement 2303′, a second terminal sequencing primer binding site 2304, a second terminal sequencing primer binding site complement 2304′, and a first terminal sequencing primer binding site 2303, these are optional as mentioned above. Accordingly, these sections may be omitted from the library.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (such as a primer-binding sequence and a sequencing primer binding site, or a combination of a primer-binding sequence, an index sequence and a sequencing primer binding site) may be referred to herein as an adaptor sequence, and an insert is flanked by a 5′ adaptor sequence and a 3′ adaptor sequence. The primer-binding sequence may also comprise a sequencing primer for the index read.

As used herein, an “adaptor” refers to a sequence that comprises a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the PS' and P7′ primer-binding sequences are complementary to short primer sequences (or lawn primers) present on the surface of a flow cell. Binding of PS' and P7′ to their complements (PS and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

### Cluster Generation and Amplification (Separate Polynucleotide Sequences)

Once a double stranded nucleic acid library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example PS and P7 lawn primers).

Thus, embodiments of the present invention may be performed on a solid support 200, such as a flowcell. However, in alternative embodiments, seeding and clustering can be conducted off-flowcell using other types of solid support.

The solid support 2200 may comprise a substrate 2204. See FIG. 28. The substrate 2204 comprises at least one well 2203 (e.g. a nanowell), and typically comprises a plurality of wells 2203 (e.g. a plurality of nanowells).

The solid support may comprise at least one first immobilised primer and at least one second immobilised primer.

Thus, each well 2203 may comprise at least one first immobilised primer 2201, and typically may comprise a plurality of first immobilised primers 2201. In addition, each well 2203 may comprise at least one second immobilised primer 2202, and typically may comprise a plurality of second immobilised primers 2202. Thus, each well 2203 may comprise at least one first immobilised primer 2201 and at least one second immobilised primer 2202, and typically may comprise a plurality of first immobilised primers 2201 and a plurality of second immobilised primers 2202.

The first immobilised primer 2201 may be attached via a 5′-end of its polynucleotide chain to the solid support 2200. When extension occurs from first immobilised primer 2201, the extension may be in a direction away from the solid support 2200.

The second immobilised primer 2202 may be attached via a 5′-end of its polynucleotide chain to the solid support 2200. When extension occurs from second immobilised primer 2202, the extension may be in a direction away from the solid support 2200.

The first immobilised primer 2201 may be different to the second immobilised primer 2202 and/or a complement of the second immobilised primer 2202. The second immobilised primer 2202 may be different to the first immobilised primer 2201 and/or a complement of the first immobilised primer 2201.

The (or each of the) first immobilised primer(s) 2201 may comprise a sequence as defined in SEQ ID NO. 1 or 5, or a variant or fragment thereof. The second immobilised primer(s) 2202 may comprise a sequence as defined in SEQ ID NO. 2, or a variant or fragment thereof. Byway of brief example, following attachment of the PS and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, SxSSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either PS' or P7′) which is capable of bridging to the second primer molecule immobilised on the solid support and binding. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products. This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

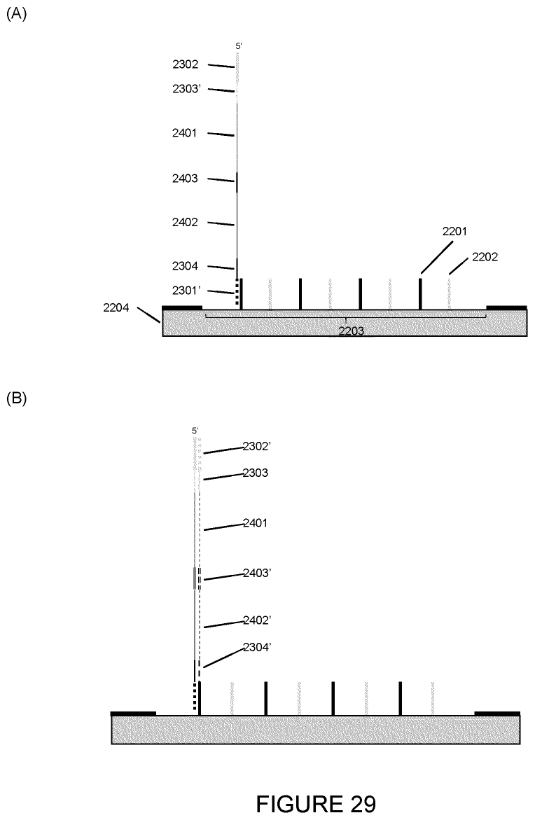

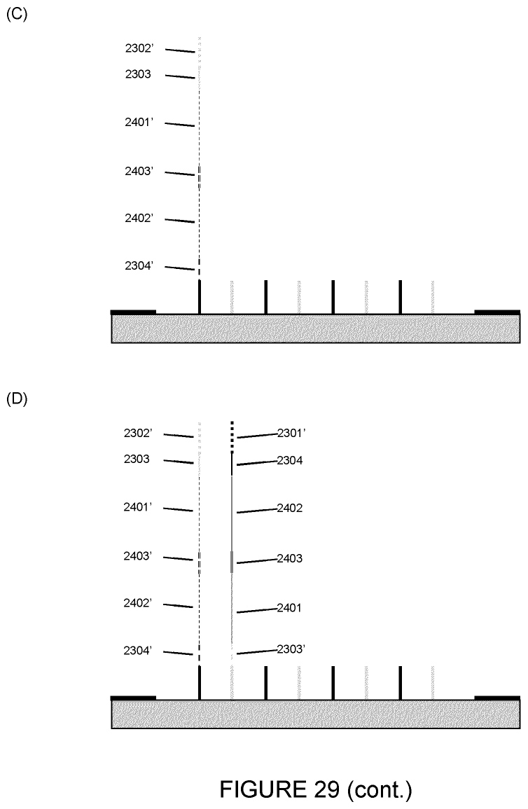

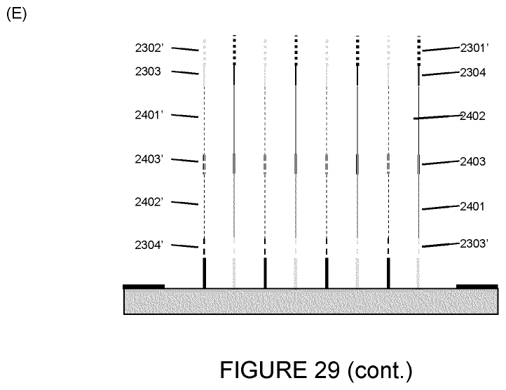

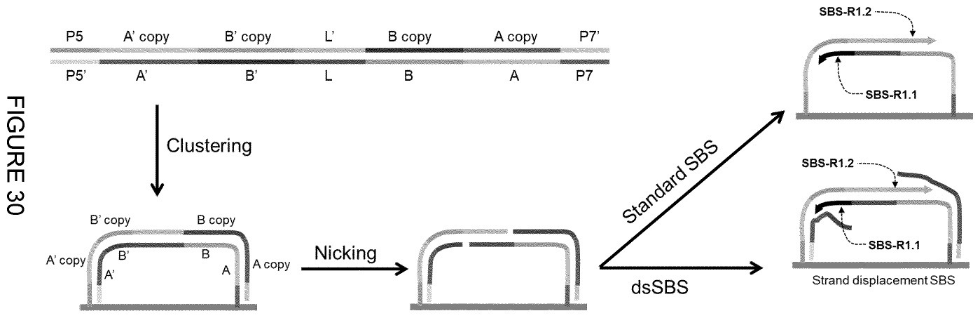

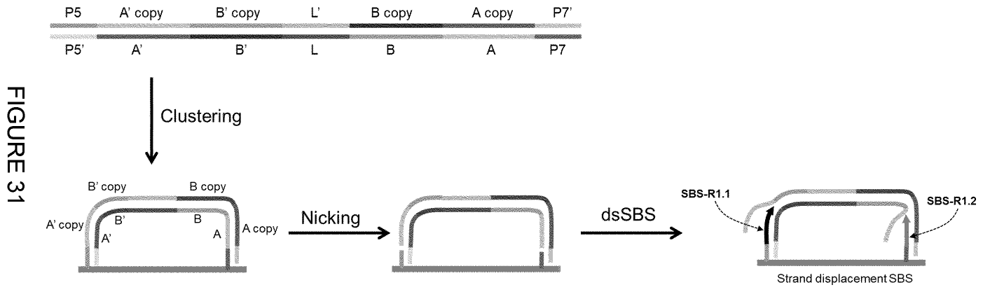

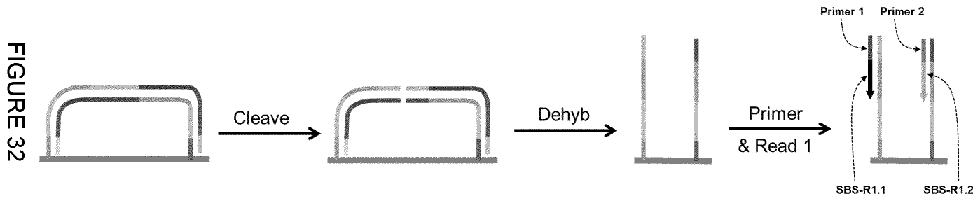

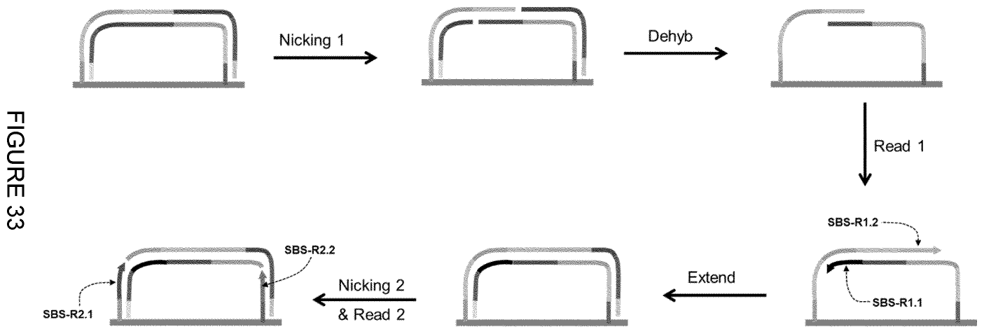

The steps of cluster generation and amplification for templates including a first polynucleotide sequence comprising a first portion and a second polynucleotide sequence comprising a second portion are illustrated below and in FIG. 29.

In cases where (separate) polynucleotide strands are used, each first polynucleotide sequence may be attached (via the 5′-end of the first polynucleotide sequence) to a first immobilised primer, and wherein each second polynucleotide sequence is attached (via the 5′-end of the second polynucleotide sequence) to a second immobilised primer. Each first polynucleotide sequence may comprise a second adaptor sequence, wherein the second adaptor sequence comprises a portion which is substantially complementary to the second immobilised primer (or is substantially complementary to the second immobilised primer). The second adaptor sequence may be at a 3′-end of the first polynucleotide sequence. Each second polynucleotide sequence may comprise a first adaptor sequence, wherein the first adaptor sequence comprises a portion which is substantially complementary to the first immobilised primer (or is substantially complementary to the first immobilised primer). The first adaptor sequence may be at a 3′-end of the second polynucleotide sequence.

In an embodiment, a solution comprising a polynucleotide library prepared by a loop fork method as described above may be flowed across a flowcell.

A particular polynucleotide strand from the polynucleotide library to be sequenced comprising, in a 5′ to 3′ direction, a second primer-binding complement sequence 2302 (e.g. P7), an optional first terminal sequencing primer binding site complement 2303′, a first insert sequence 2401 (A and B), a loop sequence 2403 (L), a second insert sequence 2402 (B′ and A′), an optional second terminal sequencing primer binding site 2304, and a first primer-binding sequence 2301′ (e.g. PS′), may anneal (via the first primer-binding sequence 2301′) to the first immobilised primer 2201 (e.g. PS lawn primer) located within a particular well 2203 (FIG. 29A).

The polynucleotide library may comprise other polynucleotide strands with different first insert sequences 2401 and second insert sequences 2402. Such other polynucleotide strands may anneal to corresponding first immobilised primers 2201 (e.g. PS lawn primers) in different wells 2203, thus enabling parallel processing of the various different strands within the polynucleotide library.

A new polynucleotide strand may then be synthesised, extending from the first immobilised primer 2201 (e.g. PS lawn primer) in a direction away from the substrate 2204. By using complementary base-pairing, this generates a template strand comprising, in a 5′ to 3′ direction, the first immobilised primer 2201 (e.g. PS lawn primer) which is attached to the solid support 2200, an optional second terminal sequencing primer binding site complement 2304′, a second insert complement sequence 2402′ (A′ copy and B′ copy), a loop complement sequence 2403′ (L′), a first insert complement sequence 2401′ (B copy and A copy), an optional first terminal sequencing primer binding site 2303, and a second primer-binding sequence 2302′ (e.g. P7′) (FIG. 29B). Such a process may utilise a polymerase, such as a DNA or RNA polymerase.

If the polynucleotides in the library comprise index sequences, then corresponding index sequences are also produced in the template.

The polynucleotide strand from the polynucleotide library may then be dehybridised and washed away, leaving a template strand attached to the first immobilised primer 2201 (e.g. PS lawn primer) (FIG. 29C).

The second primer-binding sequence 2302′ (e.g. P7′) on the template strand may then anneal to a second immobilised primer 2202 (e.g. P7 lawn primer) located within the well 2203. This forms a “bridge”.

A new polynucleotide strand may then be synthesised by bridge amplification, extending from the second immobilised primer 2202 (e.g. P7 lawn primer) (initially) in a direction away from the substrate 2204. By using complementary base-pairing, this generates a template strand comprising, in a 5′ to 3′ direction, the second immobilised primer 2202 (e.g. P7 lawn primer) which is attached to the solid support 2200, an optional first terminal sequencing primer binding site complement 2303′, a first insert sequence 2401 (A and B), a loop sequence 2403 (L), a second insert sequence 2402 (B′ and A′), an optional second terminal sequencing primer binding site 2304, and a first primer-binding sequence 2301′ (e.g. PS′). Again, such a process may utilise a polymerase, such as a DNA or RNA polymerase.

The strand attached to the second immobilised primer 2202 (e.g. P7 lawn primer) may then be dehybridised from the strand attached to the first immobilised primer 2201 (e.g. PS lawn primer) (FIG. 29D).

A subsequent bridge amplification cycle can then lead to amplification of the strand attached to the first immobilised primer 2201 (e.g. PS lawn primer) and the strand attached to the second immobilised primer 2202 (e.g. P7 lawn primer). The second primer-binding sequence 2302′ (e.g. P7′) on the template strand attached to the first immobilised primer 2201 (e.g. PS lawn primer) may then anneal to another second immobilised primer 2202 (e.g. P7 lawn primer) located within the well 2203. In a similar fashion, the first primer-binding sequence 2301′ (e.g. PS′) on the template strand attached to the second immobilised primer 2202 (e.g. P7 lawn primer) may then anneal to another first immobilised primer 2201 (e.g. PS lawn primer) located within the well 2203.

Completion of bridge amplification and dehybridisation may then provide an amplified cluster, thus providing a plurality of polynucleotide sequences comprising a first insert complement sequence 2401′ and a second insert complement sequence 2402′, as well as a plurality of polynucleotide sequences comprising a first insert sequence 2401 and a second insert sequence 2402 (FIG. 29E).

If desired, further bridge amplification cycles may be conducted to increase the number of polynucleotide sequences within the well 2203.

Once again, although FIG. 29 shows the presence of a first terminal sequencing primer binding site complement 2303′, a second terminal sequencing primer binding site 2304, a second terminal sequencing primer binding site complement 2304′, and a first terminal sequencing primer binding site 2303, these are optional as mentioned above. Accordingly, these sections may be omitted from the template and template complement strands.

### Sequencing (Separate Polynucleotide Sequences)

In one embodiment, the sequencing process comprises a first sequencing read and second sequencing read. The first sequencing read and the second sequencing read may be conducted concurrently. In other words, the first sequencing read and the second sequencing read may be conducted at the same time.

The first sequencing read may comprise the binding of a first sequencing primer (also known as a read 1.1 sequencing primer) to the first sequencing primer binding site (e.g. within loop complement sequence 2403′). The second sequencing read may comprise the binding of a second sequencing primer (also known as a read 1.2 sequencing primer) to the second sequencing primer binding site (e.g. within loop sequence 2403).

This leads to sequencing of the first portion (e.g. second insert complement sequence 2402′) and the second portion (e.g. first insert sequence 2401).

Other embodiments may involve strand displacement sequencing-by-synthesis (strand displacement SBS). In such a case, a strand displacement polymerase may initiate SBS from a nick. Further examples of strand displacement SBS are described in greater detail below.

Alternative methods of sequencing include sequencing by ligation, for example as described in U.S. Pat. No. 6,306,597 or WO 06/084132, the contents of which are incorporated herein by reference.

### Sequencing of Modified Cytosines (Separate Polynucleotide Molecules)

In one example, polynucleotide sequences may be prepared for detection of modified cytosines by a method comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template,
wherein the template is generated from a (double-stranded) target polynucleotide to be sequenced via complementary base pairing, and wherein the target polynucleotide has been pre-treated using a conversion reagent,
wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil.

As described herein, the polynucleotide sequences may each comprise portions of a double-stranded nucleic acid template, and the first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). Alternatively, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand).

The first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced.

The template is generated from a (double-stranded) target polynucleotide to be sequenced via complementary base pairing. The (double-stranded) target polynucleotide may be one (double-stranded) polynucleotide present in a polynucleotide library to be sequenced. As such, the template allows sequence information to be obtained for that particular polynucleotide.

The method may further comprise a step of preparing the first portion and the second portion for concurrent sequencing.

For example, the method may comprise simultaneously contacting first sequencing primer binding sites located after a 3′-end of the first portions with first primers and second sequencing primer binding sites located after a 3′-end of the second portions with second primers. Thus, the first portions and second portions are primed for concurrent sequencing.

The method may alternatively or additionally comprise nicking the at least one first polynucleotide sequence and nicking the at least one second polynucleotide sequence. In some embodiments, the nick on the at least one first polynucleotide sequence may be located after a 3′-end of the first portion, and the nick on the at least one second polynucleotide sequence may be located after a 3′-end of the second portion. In some embodiments, the nick on the at least one first polynucleotide sequence may be located before a 5′-end of the first portion, and the nick on the at least one second polynucleotide sequence may be located before a 5′-end of the second portion. Thus, the first portions and second portions are primed for concurrent sequencing as sequencing may begin from the nick (e.g. by using strand displacement SBS, or after washing off non-immobilised strands).

In some embodiments, a proportion of first portions may be capable of generating a first signal and a proportion of second portions may be capable of generating a second signal, wherein an intensity of the first signal is substantially the same as an intensity of the second signal.

The first signal and the second signal may be spatially unresolved (e.g. generated from the same region or substantially overlapping regions).

The first portion may be referred to herein as read 1.1 (R1.1). The second portion may be referred to herein as read 1.2 (R1.2).

The first and second strand may be separately attached to a solid support. The solid support may be a flow cell. Each of the first and second strands may be attached to the solid support (e.g. flow cell) in a single well of the solid support.

The polynucleotide strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a first polynucleotide strand and a second polynucleotide strand, the cluster formed may be a duoclonal cluster.

By “duoclonal” cluster is meant that the population of polynucleotide sequences that are then sequenced (as the next step) are substantially of two types—e.g. a first sequence and a second sequence. As such, a “duoclonal” cluster may refer to the population of single first sequences and single second sequences within a well that are then sequenced. A “duoclonal” cluster may contain a sufficient number of copies of a single first sequence and copies of a single second sequence such that the cluster is able to output a signal (e.g. a light signal) that allows sequencing reads to be performed on the “monoclonal” cluster. A “duoclonal” cluster may comprise, for example, about 500 to about 2000 combined copies, about 600 to about 1800 combined copies, about 700 to about 1600 combined copies, about 800 to 1400 combined copies, about 900 to 1200 combined copies, or about 1000 combined copies of single first sequences and single second sequences. The copies of single first sequences and single second sequences together may comprise at least about 50%, about 60%, at least about 70%, at least about 80%, at least about 90%, or about 95%, 98%, 99% or 100% of all polynucleotides within a single well of the flow cell, and thus providing a substantially duoclonal “cluster”.

The at least one first polynucleotide sequence comprising a first portion and at least one second polynucleotide sequence may be prepared using a loop fork method as described herein (see FIG. 27).

Accordingly, in one embodiment, the step of synthesising at least one first polynucleotide sequence comprising a first portion and at least one second polynucleotide sequence comprising a second portion may comprise:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide, and
synthesising the at least one second polynucleotide sequence comprising the at least one second polynucleotide sequence by forming a complement of the at least one first polynucleotide sequence.

Typically, the loop may be generated by attaching a first flanking adaptor to the target (double-stranded) polynucleotide.

The first flanking adaptor may be an oligonucleotide of any structure or any sequence that allows the forward and reverse strands to be connected via a loop. The first flanking adaptor may comprise a base-paired stem and a hairpin loop (e.g. a loop structure with unpaired or non-Watson-Crick paired nucleotides) and connect the 3′ end of the forward strand with the 5′ end of the reverse strand, or the 5′ end of the forward strand with the 3′ end of the reverse strand.

The step of synthesising the loop-ligated precursor polynucleotide may further comprise connecting a 5′-end of the forward strand of the target polynucleotide and a 3′-end of the reverse strand of the target polynucleotide (when the 3′-end of the forward strand of the target polynucleotide and the 5′-end of the reverse strand of the target polynucleotide are connected with a loop), or a 3′-end of the forward strand of the target polynucleotide and a 5′-end of the reverse strand of the target polynucleotide (when the 5′-end of the forward strand of the target polynucleotide and the 3′-end of the reverse strand of the target polynucleotide are connected with a loop), with a second flanking adaptor.

In one embodiment, the second flanking adaptor comprises a base-paired stem, a primer-binding sequence and a primer-binding complement sequence. Specifically, the second flanking adaptor may comprise a first and second strand, wherein the first and second strands are base-paired for a portion of their sequence (forming the base-paired stem) and are non-complementary for the remainder of their sequence, for example, PS' and P7 or P7′ and PS, which subsequently forms a fork structure, wherein a first arm of the fork structure comprises a primer-binding sequence and the second arm of the fork structure comprises a primer-binding complement sequence. In an alternative embodiment, the second flanking adaptor may comprise a base-paired stem and a hairpin loop, where the loop comprises a primer-binding sequence, a cleavable site and primer-binding complement sequence, where the cleavable site is in-between the primer-binding sequence and the primer-binding complement sequence. In this alternative embodiment, the method may comprise cleaving the loop of the second flanking adaptor at the cleavable site to open the loop. This will generate a fork structure, as described above. Specifically, following cleavage the second flanking adaptor will form a base-paired stem and then a fork.

As used herein for the second flanking adaptor, by “cleavable site” is meant any moiety, such as a modified nucleotide, that allows selective cleavage of the second flanking adaptor sequence. By way of non-limiting example, the cleavable site may comprise uracil bases, phosphorothioate groups, ribonucleotides, diol linkages, disulphide linkages, peptides etc.

In one example, the cleavable site is a uracil. Uracil can be cleaved using a uracil glycosylase or USER enzyme mix (which is a cocktail of uracil glycosylase and endonuclease VIII). In another example, the cleavable site is 8-oxoguanine. 8-oxoguanine can be cleaved using a FPG glycosylase. Alternatively, the cleavable site is a restriction site.

By “restriction site” is meant a sequence of nucleotides recognised by an endonuclease, for example a single-stranded endonuclease. A restriction site may also be referred to as a “recognition site” or “recognition sequence”, and such terms may be used interchangeably.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb.BbvCI, Nb.BsmI, Nb.BsrDI, Nb.BtsI, Nt.AlwI, Nt.BsmAI, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

The second flanking adaptor may comprise at least one primer-binding sequence. The second flanking adaptor may also comprise at least one primer-binding complement sequence. In some aspects, the second flanking adaptor comprises both a primer-binding sequence and a primer-binding complement sequence. The primer-binding sequence may be capable of binding to a lawn or immobilised primer that is immobilised on the surface of a solid support. For example, the primer-binding sequence may be either PS' (for example, SEQ ID NO: 3 or 6 or a variant or fragment thereof) or P7′ (for example, SEQ ID NO: 4 or a variant or fragment thereof). Similarly, the primer-binding complement sequence may be either PS (for example, SEQ ID NO: 1 or 5 or a variant or fragment thereof) or P7 (for example, SEQ ID NO: 2 or a variant or fragment thereof). If the primer-binding sequence is PS′, the primer-binding complement sequence is P7. If the primer-binding sequence is P7′, the primer-binding complement sequence is PS.

At least one of the first flanking adaptor and the second flanking adaptor comprises a restriction site for an endonuclease, such as a single-stranded endonuclease. If the second flanking adaptor comprises a base-paired stem and a hairpin loop structure, then the restriction site for an endonuclease is additional to the cleavable site. Where the restriction site is present in the first flanking adaptor, this allows a nick to be generated in the template and/or template complement strands in the loop (and/or loop complement) formed from the first flanking adaptor. Where the restriction site is present in the second flanking adaptor, this allows a nick to be generated close to the first immobilised primer and/or the second immobilised primer. Where nicking is used, such a nick prepares the strands for sequencing, since sequencing can be initiated from the nick (e.g. using strand displacement SBS), or allows non-immobilised polynucleotide sequences to be washed away to enable binding of sequencing primers.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb.BbvCI, Nb.BsmI, Nb.BsrDI, Nb.BtsI, Nt.AlwI, Nt.BsmAI, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

The first and second flanking adaptors also may comprise one or more sequencing primer-binding sites (or sequencing primer-binding site complements). The sequencing primer-binding sites and the sequencing primer-binding site complements may allow binding of a sequencing primer.

In the first flanking adaptor the sequencing primer-binding sites may be in the loop sequence or in the base-paired stem. In one embodiment, the base-paired stem comprises at least one sequencing primer-binding site. The sequencing primer-binding site may be in the base-paired stem, and in the part of the stem that connects to the reverse strand of the double-stranded polynucleotide. In another embodiment, the loop may comprise two sequencing primer-binding sites. The loop may comprise two sequencing primer-binding sites and a restriction site, wherein the sequencing primer-binding sites are either side of the restriction site.

In the second flanking adaptor the sequencing primer-binding site(s) may also be in the base-paired stem. Alternatively, each fork of the second flanking adaptor may additionally comprise a sequencing primer-binding site.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The sequence of the sequencing primers and the sequence primer binding sites are not material to the methods of the invention, as long as the sequencing primers are able to bind to the sequence primer binding site (or sequencing binding site complement) to enable amplification and sequencing of the regions to be identified.

In some embodiments, the restriction site in the first flanking adaptor is in the middle of the loop or substantially the middle of the loop. In particular, the restriction site may be cleavable by a double strand restriction endonuclease or restriction enzyme. By either of these terms is meant an enzyme that can hydrolyze both strands of the double-stranded polynucleotide (duplex), to produce polynucleotide molecules that are cleaved on both strands. The restriction enzyme may be a type II restriction enzyme.

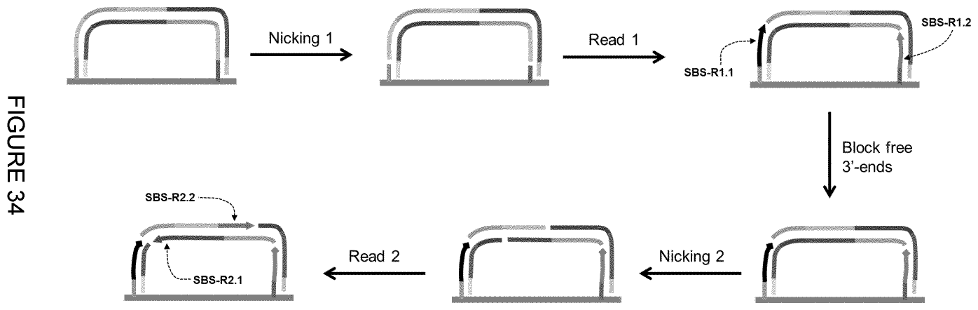

FIGS. 30 to 34 illustrate various ways in which first portions and second portions can be prepared for concurrent sequencing.

FIG. 30 shows how concurrent sequencing is enabled by nicking after a 3′-end of the first portion, and nicking after a 3′-end of the second portion. Here, the nicks are made at a 3′-end of both the loop and loop complement. In one case, non-immobilised strands may be washed away and standard SBS can be conducted, resulting in concurrent sequencing of the first and second portions. In an alternative case, the non-immobilised strands are not washed away and SBS can be conducted using a strand displacement polymerase, again resulting in concurrent sequencing of the first and second portions.

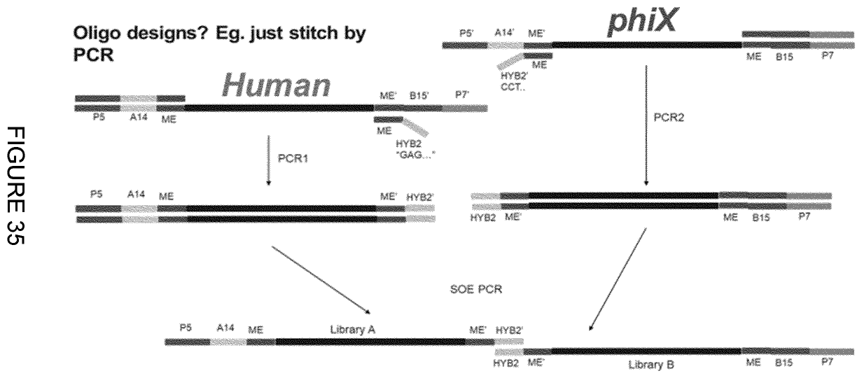

FIG. 31 shows how concurrent sequencing is enabled by nicking before a 5′-end of the first portion, and nicking before a 5′-end of the second portion. Here, the nicks are made after a 3′-end of the first immobilised primer and after a 3′-end of the second immobilised primer. SBS can then be conducted using a strand displacement polymerase, resulting in concurrent sequencing of the first and second portions.

FIG. 32 shows how concurrent sequencing is enabled by contacting first sequencing primer binding sites located after a 3′-end of the first portions with first primers and second sequencing primer binding sites located after a 3′-end of the second portions with second primers. Here, a middle portion of the loop and loop complement may be cleaved (e.g. with a double strand restriction endonuclease or restriction enzyme). The non-immobilised strands may be washed away, and any remaining sections of the loop and loop complement can act as sequencing primer binding sites, allowing standard SBS to be conducted resulting in concurrent sequencing of the first and second portions.

It is also possible to conduct paired end reads using these methods. FIGS. 33 and 34 illustrate various ways in which paired end reads can be achieved.

FIG. 33 shows paired end reads being conducted after a first round of concurrent sequencing as shown in FIG. 30. Further nicks can be made after a 3′-end of the first immobilised primer and after a 3′-end of the second immobilised primer. SBS can then be conducted using a strand displacement polymerase, resulting in concurrent sequencing of complements of the first and second portions.

FIG. 34 shows paired end reads being conducted after a first round of concurrent sequencing as shown in FIG. 31. Any free 3′-ends can be blocked. Further nicks can be made after a 3′-end of the first portion, and after a 3′-end of the second portion, then SBS can then be conducted using a strand displacement polymerase, resulting in concurrent sequencing of complements of the first and second portions.

Although not shown in FIG. 32, paired end reads can also be conducted after Read 1.1 and Read 1.2. This can be achieved by having further immobilised primers on the solid support that are substantially complementary to the remaining sections of the loop and loop complement acting as sequencing primer binding sites. This allows resynthesis of the strands, and subsequent binding of further sequencing primers for concurrent sequencing of complements of the first and second portions.

In some embodiments, the method may further comprise a step of concurrently sequencing nucleobases in the first portion and the second portion.

The target polynucleotide (or in some embodiments, the polynucleotide library) has been pre-treated using a conversion reagent. In some embodiments, the method of preparing at least one polynucleotide sequence for detection of modified cytosines may include a step of treating the target polynucleotide using a conversion agent.

### Conversion Agent Treatment

The conversion reagent is configured to convert a modified cytosine to thymine or a nucleobase which is read as thymine/uracil, and/or is configured to convert an unmodified cytosine to uracil or a nucleobase which is read as thymine/uracil.

As used herein, the term “modified cytosine” may refer to any one or more of 5-methylcytosine (5-mC), 5-hydroxymethylcytosine (5-hmC), 5-formylcytosine (5-fC) and 5-carboxylcytosine (5-caC):

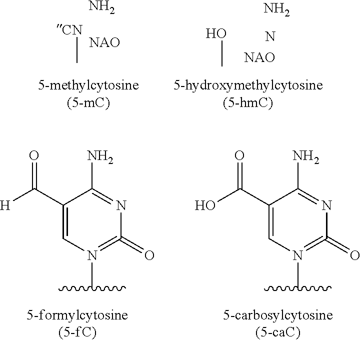

wherein the wavy line indicates an attachment point of the modified cytosine to the polynucleotide.

As used herein, the term “unmodified cytosine” refers to cytosine (C):

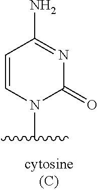 [SMILES: *n1ccc(N)nc1=O]

wherein the wavy line indicates an attachment point of the unmodified cytosine to the polynucleotide.

As used herein, the term “conversion reagent configured to convert a modified cytosine to thymine or a nucleobase which is read as thymine/uracil” may refer to a reagent which converts one or more modified cytosines (e.g. 5-methylcytosine, 5-hydroxymethylcytosine, 5-formylcytosine and 5-carboxylcytosine) to thymine (i.e. would base pair with adenine), or to an equivalent nucleobase which would base pair with adenine. The conversion may comprise a deamination reaction converting the modified cytosine to thymine or nucleobase which is read as thymine/uracil.

As used herein, the term “conversion reagent configured to convert an unmodified cytosine to uracil or a nucleobase which is read as thymine/uracil” may refer to a reagent which converts one or more unmodified cytosines to uracil (i.e. would base pair with adenine), or to an equivalent nucleobase which would base pair with adenine. The conversion may comprise a deamination reaction converting the unmodified cytosine to uracil or nucleobase which is read as thymine/uracil.

In general, if modified cytosines were present in the target polynucleotide to be sequenced, the forward strand of the template will then not be identical to the reverse complement strand of the template as a result of treatment of the target polynucleotide with the conversion agent (alternatively, the reverse strand of the template will then not be identical to the forward complement strand of the template as a result of treatment of the target polynucleotide with the conversion agent). However, if modified cytosines were not present in the target polynucleotide to be sequenced, the forward strand of the template will then be (substantially) identical to the reverse complement strand of the template despite treatment of the target polynucleotide with the conversion agent (alternatively, the reverse strand of the template will then be (substantially) identical to the forward complement strand of the template despite treatment of the target polynucleotide with the conversion agent). As such, mismatches between the forward strand of the template and the reverse complement strand of the template allow the detection of modified cytosines (alternatively, mismatches between the reverse strand of the template and the forward complement strand of the template allow detection of modified cytosines).

Where the forward strand (or reverse strand) of the template is not identical to the reverse complement strand (or forward complement strand) of the template as a result of treatment with the conversion agent, the forward strand (or reverse strand) of the template may comprise a guanine base at a first position, which leads to a basecall of C for the original target polynucleotide; and wherein the reverse complement strand (or forward complement strand) of the template may comprise an adenine base at a second position corresponding to the same position number as the first position, which leads to a basecall of T for the original target polynucleotide. The adenine base at the second position within the template may have been generated as a result of conversion of modified cytosines in the target polynucleotide to thymine, or to an equivalent nucleobase which would base pair with adenine; or may have been generated as a result of conversion of unmodified cytosines in the target polynucleotide to uracil, or to an equivalent nucleobase which would base pair with adenine. In particular, the adenine base at the second position within the template may have been generated as a result of conversion of unmodified cytosines in the target polynucleotide to uracil, or to an equivalent nucleobase which would base pair with adenine.

In other cases, the forward strand (or reverse strand) of the template comprises an adenine base at a first position, which leads to a basecall of T for the original target polynucleotide; and wherein the reverse complement strand (or forward complement strand) of the template comprises a guanine base at a second position corresponding to the same position number as the first position, which leads to a basecall of C for the original target polynucleotide. Similarly, the adenine base at the first position within the template may have been generated as a result of conversion of modified cytosines in the target polynucleotide to thymine, or to an equivalent nucleobase which would base pair with adenine; or may have been generated as a result of conversion of unmodified cytosines in the target polynucleotide to uracil, or to an equivalent nucleobase which would base pair with adenine. In particular, the adenine base at the first position within the template may have been generated as a result of conversion of modified cytosines in the target polynucleotide to thymine, or to an equivalent nucleobase which would base pair with adenine.

In some embodiments, the conversion reagent configured to convert a modified cytosine to thymine or a nucleobase which is read as thymine/uracil may further be configured to be selective for converting one or more modified cytosines (e.g. 5-methylcytosine, 5-hydroxymethylcytosine, 5-formylcytosine and 5-carboxylcytosine) over converting unmodified cytosine. The selectivity may be measured by comparing reaction parameters (e.g. deamination reaction parameters) of the conversion of a particular modified cytosine to thymine or equivalent nucleobase which is read as thymine/uracil, with corresponding reaction parameters (e.g. deamination reaction parameters) of the conversion of unmodified cytosine to uracil or nucleobase which is read as thymine/uracil. For example, reaction parameters such as rate of reaction or yield may be compared. In the case of rate of reaction, a rate of a reaction (e.g. deamination) of the particular modified cytosine to thymine or nucleobase which is read as thymine/uracil may be greater (e.g. at least 2 times greater, at least 5 times greater, at least 10 times greater, at least 20 times greater, at least 50 times greater, or at least 100 times greater) than a corresponding rate of a reaction (e.g. deamination) of the unmodified cytosine to uracil or nucleobase which is read as thymine/uracil. In the case of yield, a yield of a reaction (e.g. deamination) of the particular modified cytosine to thymine or nucleobase which is read as thymine/uracil may be greater (e.g. at least 2 times greater, at least 5 times greater, at least 10 times greater, at least 20 times greater, at least 50 times greater, or at least 100 times greater) than a corresponding yield of a reaction (e.g. deamination) of the unmodified cytosine to uracil or nucleobase which is read as thymine/uracil.

In some embodiments, the conversion reagent configured to convert an unmodified cytosine to uracil or a nucleobase which is read as thymine/uracil may further be configured to be selective for converting unmodified cytosine over converting one or more modified cytosines (e.g. 5-methylcytosine, 5-hydroxymethylcytosine, 5-formylcytosine and 5-carboxylcytosine). The selectivity may be measured by comparing reaction parameters (e.g. deamination reaction parameters) of the conversion of unmodified cytosine to uracil or nucleobase which is read as thymine/uracil, with corresponding reaction parameters (e.g. deamination reaction parameters) of the conversion of a particular modified cytosine to thymine or nucleobase which is read as thymine/uracil. For example, reaction parameters such as rate of reaction or yield may be compared. In the case of rate of reaction, a rate of a reaction (e.g. deamination) of the unmodified cytosine to uracil or nucleobase which is read as thymine/uracil may be greater (e.g. at least 2 times greater, at least 5 times greater, at least 10 times greater, at least 20 times greater, at least 50 times greater, or at least 100 times greater) than a rate of a reaction (e.g. deamination) of the particular modified cytosine to uracil or the nucleobase which is read as thymine/uracil. In the case of yield, a yield of a reaction (e.g. deamination) the unmodified cytosine to uracil or nucleobase which is read as thymine/uracil may be greater (e.g. at least 2 times greater, at least 5 times greater, at least 10 times greater, at least 20 times greater, at least 50 times greater, or at least 100 times greater) than a corresponding yield of a reaction (e.g. deamination) of the particular modified cytosine to uracil or the nucleobase which is read as thymine/uracil.

In one embodiment, the conversion agent may comprise a chemical agent and/or an enzyme.

In one embodiment, the chemical agent may comprise a boron-based reducing agent. In one aspect, the boron-based reducing agent is an amine-borane compound or an azine-borane compound (wherein the term “azine” refers to a nitrogenous heterocyclic compound comprising a 6-membered aromatic ring). Non-limiting examples of amine-borane compounds include compounds such as t-butylamine borane, ammonia borane, ethylenediamine borane and dimethylamine borane. Non-limiting examples of azine-borane compounds include compounds such as pyridine borane and 2-picoline borane.

In general, boron-based reducing agents are able to convert 5-formylcytosine and 5-carboxylcytosine to dihydrouracil (i.e. a nucleobase which is read as thymine/uracil). The reaction proceeds by reduction of the internal C═C bond of 5-formylcytosine or 5-carboxylcytosine, deamination, and then decarboxylation to form dihydrouracil (illustrated below using 5-carboxylcytosine):

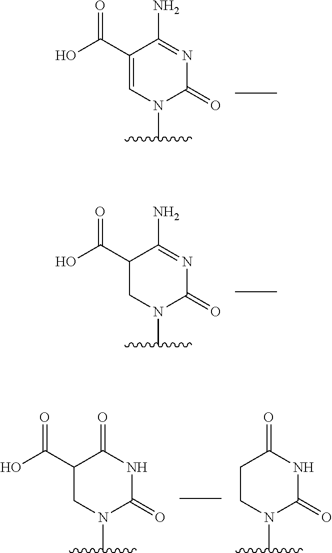 [SMILES: *n1cc(C(=O)O)c(N)nc1=O ; *N1CC(C(=O)O)C(N)=NC1=O ; *N1CC(C(=O)O)C(=O)NC1=O ; *N1CCC(=O)NC1=O]

This process is selective for a particular type of modified cytosine (5-carboxylcytosine) and does not convert unmodified cytosine. Where distinction between other modified cytosines and unmodified cytosines is desired (or even between different types of modified cytosines), treatment with further agents as described herein prior to treatment with the boron-based reducing agent may provide such distinction. In particular, boron-based reducing agents may be combined with ten-eleven translocation (TET) methylcytosine dioxygenases, 13-glucosyltransferases, oxidising agents, oximes and/or hydrazones as described herein.

In one embodiment, the chemical agent may comprise sulfite. The sulfite may be present in a partially acid/salt form (e.g. as bisulfite ions), or be present in a salt form (e.g. as sulfite ions). In cases where the sulfite is present in a salt form, the sulfite may comprise a cation (not including W). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts). The term “sulfite” also encompasses “metabisulfite”, which dissolves in aqueous solution to form bisulfite.

In general, sulfite (e.g. bisulfite) is able to convert unmodified cytosine to uracil. The reaction proceeds via conjugate addition of sulfite to the internal C═C of unmodified cytosine, deamination, and then elimination of sulfite to reform the internal C═C bond to form uracil:

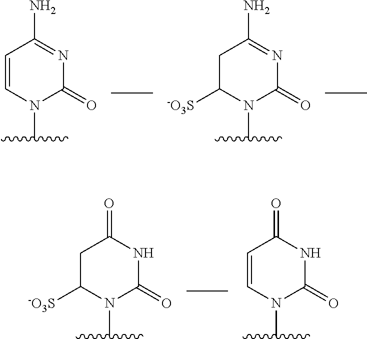

This process is selective for unmodified cytosine over certain types of modified cytosine (5-methylcytosine and 5-hydroxymethylcytosine). However, 5-formylcytosine and 5-carboxylcytosine are converted to their equivalent deaminated versions. Where distinction between different types of modified cytosines (e.g. 5-formylcytosine and 5-carboxylcytosine) is desired, treatment with further agents as described herein prior to treatment with the sulfite may provide such distinction. In particular, the sulfite may be combined with ten-eleven translocation (TET) methylcytosine dioxygenases, 13-glucosyltransferases, oxidising agents and/or reducing agents as described herein.

In one embodiment, the enzyme may comprise a cytidine deaminase.

As used herein, the term “cytidine deaminase” may refer to an enzyme which is able to catalyse the following reaction:

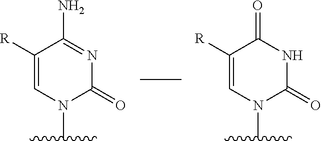

wherein R is hydrogen, methyl, hydroxymethyl, formyl or carboxyl, and wherein the wavy line indicates an attachment point to a polynucleotide.

The cytidine deaminase may be a wild-type cytidine deaminase or a mutant cytidine deaminase, for example a mutant cytidine deaminase.

In one embodiment, the cytidine deaminase is a member of the APOBEC protein family. The cytidine deaminase may be a member of the AID subfamily, the APOBEC1 subfamily, the APOBEC2 subfamily, the APOBEC3 subfamily (e.g. the APOBEC3A subfamily, the APOBEC3B subfamily, the APOBEC3C subfamily, the APOBEC3D subfamily, the APOBEC3F subfamily, the APOBEC3G subfamily, or the APOBEC3H subfamily), or the APOBEC4 subfamily; such as the APOBEC3A subfamily.

In general, cytidine deaminases are able to catalyse the deamination of all modified cytosines (particularly 5-methylcytosine, 5-hydroxymethylcytosine and 5-formylcytosine) to their equivalent deaminated versions (i.e. nucleobases which are read as thymine/uracil), as well as catalysing the deamination of unmodified cytosines to uracil. Nevertheless, rates of reaction may differ depending on the type of modified cytosine; for example, wild-type APOBEC3A catalyses the deamination of unmodified cytosine and 5-methylcytosine relatively efficiently, whereas deamination of 5-hydroxymethylcytosine is ˜5000-fold slower relative to unmodified cytosine, deamination of 5-formylcytosine is ˜3700-fold slower relative to unmodified cytosine, and deamination of 5-carboxylcytosine is >20000-fold slower relative to unmodified cytosine. Where distinction between modified cytosines and unmodified cytosines is desired (or even between different types of modified cytosines), treatment with further agents as described herein prior to treatment with the cytidine deaminase may provide such distinction. In particular, the cytidine deaminase may be combined with ten-eleven translocation (TET) methylcytosine dioxygenases and/or 13-glucosyltransferases as described herein. Alternatively, or in addition, particular cytidine deaminases (e.g. mutant cytidine deaminases) may be chosen which have higher affinities for modified cytosines as substrates over unmodified cytosines, or vice versa.

The APOBEC protein family is a member of the large cytidine deaminase superfamily that contains a canonical zinc-dependent deaminase (ZOO) signature motif embedded within a core cytidine deaminase fold. This fold includes a five-stranded mixed beta (b)-sheet surrounded by six alpha (a)-helices with the order a1-b1-b2-a2-b3-a3-b4-a4-b5-a5-a6 (Salter et al., Trends Biochem Sci. 2016 41(7):578-594. doi:10.1016j.tibs.2016.05.001; Salter et al., Trends Biochem. Sci. 2018, 43(8):606-622 doi.org/10.1016/j.tibs.2018.04.013). Each cytidine deaminase domain core structure of APOBEC proteins contains a highly conserved spatial arrangement of the catalytic centre residues of a zinc-binding motif H-[P/A/V]-E-X[23-2srP—C-X[2-4i-C (SEQ ID NO: 67) (referred to herein as the ZOO motif, where X is any amino acid, and the subscript range of numbers after X refers to the number of amino acids) (Salter et al., Trends Biochem Sci. 2016 41(7):578-594. doi:10.1016/j.tibs.2016.05.001). Without intending to be limited by theory, the H and two C residues coordinate a Zn atom, and the E residue polarises a water molecule near the Zn-atom for catalysis (Chen et al., 2021, Viruses, 13:497, doi.org/10.3390/v13030497).

Some members of the APOBEC protein family, e.g., the AID subfamily, the APOBEC1 subfamily, the APOBEC2 subfamily, the APOBEC3A subfamily, the APOBEC3C subfamily, the APOBEC3H subfamily, and the APOBEC4 subfamily, include one copy of the ZOO motif. Other members of the APOBEC protein family, e.g., the APOBEC3B subfamily, the APOBEC3D subfamily, the APOBEC3F subfamily, and the APOBEC3G subfamily, include two copies of the ZOO motif, but often only the C-terminal copy is active (Salter et al., Trends Biochem Sci. 2016 41(7):578-594. doi:10.1016/j.tibs.2016.05.001). Thus, a mutant cytidine deaminase disclosed herein includes one or two ZOO motifs. In one embodiment, a mutant cytidine deaminase based on a member of the APOBEC3A subfamily includes the following ZOO motif-HXEX24SW(S/T)PCX[2-41CX5FXsLXsR(UI)YX[s-111LX2LX[101M (SEQ ID NO. 68) (where X is any amino acid, and the subscript number or range of numbers after X refers to the number of amino acids) (Salter et al., Trends Biochem Sci. 2016 41(7):578-594. doi:10.1016/j.tibs.2016.05.001). Non-limiting examples of wild-type cytidine deaminases in the APOBEC protein family are shown in the table below (from UniProt, database of protein sequence and functional information, available at uniprot.org; or GenBank, collection of nucleotide sequences and their protein translations, available at ncbi.nlm.nih.gov/protein/):

| APOBEC protein | Non-limiting examples |
|---|---|
| AID | UniProt: Q9GZX7 (SEQ ID NO: 23); |
| | UniProt: G3QLD2 (SEQ ID NO: 24); |
| | Uniprot Q9WVEO (SEQ ID NO: 25) |
| APOBEC1 | UniProt: P41238 (SEQ ID NO: 26); |
| | NCBI XP_030856728.1 (SEQ ID NO: 27); |
| | Uniprot P51908 (SEQ ID NO: 28) |
| APOBEC2 | UniProt: Q9Y235 (SEQ ID NO: 29); |
| | Uniprot G3SGN8 (SEQ ID NO: 30); |
| | Uniprot Q9Wv35 (SEQ ID NO: 31) |
| APOBEC3A | UniProt: P31941(SEQ ID NO: 32); |
| | GenBank: XP_045219544.1 (SEQ ID NO: 33); |
| | GenBank: AER45717.1 (SEQ ID NO: 34); |
| | GenBank: XP_003264816.1 (SEQ ID NO: 35); |
| | GenBank: PNI48846.1 (SEQ ID NO: 36); |
| | GenBank: ADO85886.1 (SEQ ID NO: 37) |
| APOBEC3B | UniProt: Q9UH17 (SEQ ID NO: 38); |
| | Uniprot G3QV16 (SEQ ID NO: 39); |
| | Uniprot F6M3K5 (SEQ ID NO: 40) |
| APOBEC3C | UniProt: Q9NRW3 (SEQ ID NO: 41); |
| | Uniprot Q694B5 (SEQ ID NO: 42); |
| | Uniprot B0LW74 (SEQ ID NO: 43) |
| APOBEC3D | UniProt: Q96AK3 (SEQ ID NO: 44); |
| | NCBI NP_001332895.1 (SEQ ID NO: 45); |
| | NCBI NP_001332931.1 (SEQ ID NO: 46) |
| APOBEC3F | UniProt: Q8IUX4 (SEQ ID NO: 47); |
| | Uniprot G3RD21 (SEQ ID NO: 48); |
| | Uniprot Q1G0Z6 (SEQ ID NO: 49) |
| APOBEC3G | UniProt: Q9HC16 (SEQ ID NO: 50); |
| | Uniprot Q694C1 (SEQ ID NO: 51); |
| | Uniprot U5NDB3 (SEQ ID NO: 52) |
| APOBEC3H | UniProt: Q6NTF7 (SEQ ID NO: 53); |
| | Uniprot B7T0U7 (SEQ ID NO: 54); |
| | Uniprot Q19Q52 (SEQ ID NO: 55) |
| APOBEC4 | UniProt: Q8WW27(SEQ ID NO: 56); |
| | NCBI XP_004028087.1 (SEQ ID NO: 57); |
| | Uniprot Q497M3 (SEQ ID NO: 58) |

The mutant cytidine deaminase may comprise amino acid substitution mutations at positions functionally equivalent to (Tyr/Phe)130 and Tyr132 in a wild-type APOBEC3A protein. Such mutant cytidine deaminases are described in further detail in U.S. Provisional Application 63/328,444, which is incorporated herein by reference. By “functionally equivalent” it is meant that the mutant cytidine deaminase has the amino acid substitution at the amino acid position in a reference (wild-type) cytidine deaminase that has the same functional role in both the reference (wild-type) cytidine deaminase and the mutant cytidine deaminase.

The (Tyr/Phe)130 may be Tyr130, and the wild-type APOBEC3A protein may be SEQ ID NO: 32.

In one embodiment, the mutant cytidine deaminase may convert 5-methylcytosine to thymine by deamination at a greater rate than conversion rate of cytosine to uracil by deamination; such as wherein the rate is at least 100-fold greater.

The substitution mutation at the position functionally equivalent to Tyr130 may comprise Ala, Val or Trp.

The substitution mutation at the position functionally equivalent to Tyr132 may comprise a mutation to His, Arg, Gin or Lys.

The mutant cytidine deaminase may comprise a ZOO motif H-[P/AN]-E-X[23-2srP—C-X[2-4i-C (SEQ ID NO: 67).

The mutant cytidine deaminase may be a member of the APOBEC3A subfamily and may comprise a ZOO motif HXEX24SW(S/T)PCX[2-41CXGFXsLXsR(UI)YX[s-111LX2LX[101M (SEQ ID NO: 88).

In one embodiment, the target polynucleotide may be treated with a further agent prior to treatment with the conversion reagent.

The further agent may be configured to convert a modified cytosine (e.g. one of 5-methylcytosine, 5-hydroxymethylcytosine, 5-formylcytosine and 5-carboxylcytosine) to another modified cytosine (e.g. another one of 5-methylcytosine, 5-hydroxymethylcytosine, 5-formylcytosine and 5-carboxylcytosine).

For example, the further agent may be configured to convert 5-methylcytosine to 5-hydroxymethylcytosine. In the same or other embodiments, the further agent may be configured to convert 5-hydroxymethylcytosine to 5-formylcytosine. In the same or other embodiments, the further agent may be configured to convert 5-formylcytosine to 5-carboxylcytosine. In some embodiments, the further agent may be configured to convert 5-methylcytosine to 5-hydroxymethylcytosine, 5-hydroxymethylcytosine to 5-formylcytosine, and 5-formylcytosine to 5-carboxylcytosine.

In other embodiments, the further agent may be configured to convert 5-formylcytosine to 5-hydroxymethylcytosine.

The further agent may be configured to convert a modified cytosine (e.g. one of 5-methylcytosine, 5-hydroxymethylcytosine, 5-formylcytosine and 5-carboxylcytosine) to another modified cytosine (e.g. another (different) one of 5-methylcytosine, 5-hydroxymethylcytosine, 5-formylcytosine and 5-carboxylcytosine) may comprise a chemical agent and/or an enzyme.

The further agent configured to convert a modified cytosine to another modified cytosine may be a chemical agent; such as an oxidising agent; a metal-based oxidising agent; a transition metal-based oxidising agent; a ruthenium-based oxidising agent. The oxidising agent may be configured to convert 5-hydroxymethylcytosine to 5-formylcytosine. Non-limiting examples of the oxidising agent include ruthenate (e.g. potassium ruthenate, K2RuQ4), or perruthenate (e.g. potassium perruthenate, KRuQ4).

The further agent configured to convert a modified cytosine to another modified cytosine may be a chemical agent; such as a reducing agent; a Group Ill-based reducing agent; a boron-based reducing agent. The oxidising agent may be configured to convert 5-formylcytosine to 5-hydroxymethylcytosine. Non-limiting examples of the reducing agent include borohydride (e.g. sodium borohydride, lithium borohydride), or triacetoxyborohydride (e.g. sodium triacetoxyborohydride).

The further agent configured to convert a modified cytosine to another modified cytosine may be an enzyme; a ten-eleven translocation (TET) methylcytosine dioxygenase; such as wherein the TET methylcytosine dioxygenase is a member of the TET1 subfamily, the TET2 subfamily, or the TET3 subfamily. The enzyme may be configured to convert 5-methylcytosine to 5-hydroxymethylcytosine, 5-hydroxymethylcytosine to 5-formylcytosine, and 5-formylcytosine to 5-carboxylcytosine. Non-limiting examples of the TET methylcytosine dioxygenase include:

| TET protein | Non-limiting examples |
|---|---|
| TET1 | UniProt: Q8NFU7 (SEQ ID NO: 59) |
| | UniProt: Q3URK3 (SEQ ID NO: 60) |
| TET2 | UniProt: Q6N021 (SEQ ID NO: 61) |
| | UniProt: Q4JK59 (SEQ ID NO: 62) |
| TET3 | UniProt: 043151 (SEQ ID NO: 63) |
| | UniProt: Q8BG87 (SEQ ID NO: 64) |

The further agent may be configured to reduce/prevent deamination of a particular modified cytosine (e.g. one of 5-methylcytosine, 5-hydroxymethylcytosine, 5-formylcytosine and 5-carboxylcytosine). Such a further agent configured to reduce/prevent deamination of a particular modified cytosine may be used in combination with a further agent configured to convert a modified cytosine to another modified cytosine.

For example, the further agent may be configured to convert 5-hydroxymethylcytosine to a 5-hydroxymethylcytosine analogue bearing a hydroxyl protecting group. The 5-hydroxymethylcytosine analogue bearing the hydroxyl protecting group may be resistant to oxidation to form 5-formylcytosine. Non-limiting examples of hydroxyl protecting groups include sugar groups (e.g. glycosyl), silyl ether groups (e.g. trimethylsilyl, triethylsilyl, triisopropylsilyl, t-butyl(dimethyl)silyl, t-butyl(diphenyl)silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl).

In other embodiments, the further agent may be configured to convert 5-formylcytosine to a 5-formylcytosine analogue bearing an oxime or a hydrazone group. The 5-formylcytosine analogue bearing the oxime or hydrazone group may be resistant to oxidation to form 5-carboxylcytosine.

The further agent may be configured to reduce/prevent deamination of a particular modified cytosine (e.g. one of 5-methylcytosine, 5-hydroxymethylcytosine, 5-formylcytosine and 5-carboxylcytosine) may comprise a chemical agent and/or an enzyme.

The further agent configured to reduce/prevent deamination of a particular modified cytosine may be an enzyme; a glycosyltransferase (e.g. a-glucosyltransferase or 13-glucosyltransferase); a 13-glucosyltransferase. Such a further agent may be configured to convert 5-hydroxymethylcytosine to a 5-hydroxymethylcytosine analogue bearing a hydroxyl protecting group, wherein the hydroxyl protecting group is glycosyl. A non-limiting example of the enzyme includes T4-13GT, for example as supplied by New England Biolabs (catalog #M0357S, M0357L) or by ThermoFisher Scientific (catalog #E00831); further non-limiting examples of glycosyltransferases include:

| Glucosyltransferase | Non-limiting examples |
|---|---|
| a-glucosyltransferase | UniProt: P04519 (SEQ ID NO: 65) |
| 13-glucosyltransferase | UniProt: P04547 (SEQ ID NO: 66) |

The further agent configured to reduce/prevent deamination of a particular modified cytosine may be a chemical agent; a hydroxylamine or a hydrazine. Such a further agent may be configured to convert 5-formylcytosine to a 5-formylcytosine analogue bearing an oxime or a hydrazone group. Non-limiting examples of hydroxylamines include O-alkylhydroxylamines (e.g. O-methylhydroxylamine, O-ethylhydroxylamine), O-arylhydroxylamines (e.g. O-phenylhydroxylamine). Non-limiting examples of hydrazines include acylhydrazides (e.g. acethydrazide, benzhydrazide), alkylsulfonylhydrazides (e.g. methylsulfonylhydrazide), or arylsulfonylhydrazides (e.g. benzenesulfonylhydrazide, p-toluenesulfonylhydrazide).

Specific methods of modified cytosine sequencing using conversion agents (optionally combined with further agents) are further illustrated below. However, the type of conversion agents and/or further agents are not limited thereto.

### BS-Seq

Bisulfite sequencing (BS-seq) involves using bisulfite as the conversion agent. This process is described in Frommer et al. (Proc. Natl. Acad. Sci. U.S.A., 1992, 89, pp. 1827-1831), which is incorporated herein by reference. This process converts unmodified cytosines in the target polynucleotide to uracil, as well as 5-formylcytosine and 5-carboxylcytosine to deaminated analogues, but does not convert 5-methylcytosine and 5-hydroxymethylcytosine. Accordingly, BS-seq allows identification of the modified cytosines 5-mC and 5-hmC by reading them as C; whereas unmodified C, 5-fC and 5-caC are converted to nucleobases which are read as T/U.

### OxBS-Seq

Oxidative bisulfite sequencing (oxBS-seq) involves using potassium perruthenate as the further agent and bisulfite as the conversion agent. This process is described in Booth et al. (Science, 2012, 336, pp. 934-937), which is incorporated herein by reference. Potassium perruthenate causes oxidation of 5-hydroxymethylcytosine in the target polynucleotide to 5-formylcytosine. Subsequent treatment with bisulfite converts unmodified cytosines in the target polynucleotide to uracil, as well as 5-formylcytosine (including residues that used to be 5-hydroxymethylcytosine) and 5-carboxylcytosine to deaminated analogues, but does not convert 5-methylcytosine. Accordingly, oxBS-seq allows identification of the modified cytosine 5-mC by reading them as C; whereas unmodified C, 5-hmC, 5-fC and 5-caC are converted to nucleobases which are read as T/U.

### RedBS-Seq

Reduced bisulfite sequencing (redBS-seq) involves using sodium borohydride as the further agent and bisulfite as the conversion agent. This process is described in Booth et al. (Nat. Chem., 2014, 6, pp. 435-440), which is incorporated herein by reference. Sodium borohydride causes reduction of 5-formylcytosine in the target polynucleotide to 5-hydroxymethylcytosine. Subsequent treatment with bisulfite converts unmodified cytosines in the target polynucleotide to uracil, as well as 5-carboxylcytosine to its deaminated analogue, but does not convert 5-hydroxymethylcytosine (including residues that used to be 5-formylcytosine) and 5-methylcytosine. Accordingly, redBS-seq allows identification of the modified cytosines 5-mC, 5-hmC and 5-fC by reading them as C: whereas unmodified C and 5-caC are converted to nucleobases which are read as T/U.

### TAB-Seq

TET-assisted bisulfite sequencing (TAB-seq) involves using a T4 bacteriophage 13-glucosyltransferase and a TET1 enzyme as the further agents and bisulfite as the conversion agent. This process is described in Yu et al. (Cell, 2012, 149, pp. 1368-1380), which is incorporated herein by reference. The T4 bacteriophage 13-glucosyltransferase converts 5-hydroxymethylcytosine in the target polynucleotide to 13-glucosyl-5-hydroxymethylcytosine, which prevents oxidation. TET1 enzyme causes oxidation of 5-methylcytosine and 5-formylcytosine in the target polynucleotide to 5-carboxylcytosine. Subsequent treatment with bisulfite converts unmodified cytosines in the target polynucleotide to uracil, as well as 5-carboxylcytosine (including residues that used to be 5-methylcytosine and 5-formylcytosine) to its deaminated analogue, but does not convert 13-glucosyl-5-hydroxymethylcytosine. Accordingly, TAB-seq allows identification of the modified cytosine 5-hmC (as the protected glycosyl residue) by reading it as C; whereas unmodified C, 5-mC, 5-fC and 5-caC are converted to nucleobases which are read as T/U.

### ACE-Seq

APOBEC-coupled epigenetic sequencing (ACE-seq) involves using a T4 bacteriophage 13-glucosyltransferase as a further agent and APOBEC3A as the conversion agent. This process is described in Schutsky et al. (Nat. Biotechnol., 2018, 36, pp. 1083-1090), which is incorporated herein by reference. The T4 bacteriophage 13-glucosyltransferase converts 5-hydroxymethylcytosine in the target polynucleotide to 13-glucosyl-5-hydroxymethylcytosine, which prevents oxidation. Subsequent treatment with APOBEC3A converts unmodified cytosines in the target polynucleotide to uracil, as well as 5-methylcytosine to its deaminated analogue. 5-formylcytosine is also able to convert to its deaminated analogue, but reacts slower relative to unmodified cytosine and 5-methylcytosine. 5-carboxylcytosine is also able to convert to its deaminated analogue, but reacts far slower than unmodified cytosine and 5-methylcytosine, and slower than 5-formylcytosine. Accordingly, ACE-seq allows identification of the modified cytosine 5-hmC (as the protected glycosyl residue) by reading it as C; whereas unmodified C and 5-mC are converted to nucleobases which are read as T/U; 5-fC is converted to a nucleobase which is read as T/U to a limited extent; 5-caC is converted to a nucleobase which is read as T/U to a more limited extent.

### EM-Seq

Enzymatic Methyl sequencing (EM-seq) involves using T4 bacteriophage 13-glucosyltransferase and a TET2 enzyme as the further agents and APOBEC3A as the conversion agent. This process is described in Vaisvila et al. (Genome Res. 2021, 31, pp. 1280-1289), U.S. Pat. Nos. 10,619,200 B2 and 9,121,061 B2, which are incorporated herein by reference. The T4 bacteriophage 13-glucosyltransferase converts 5-hydroxymethylcytosine in the target polynucleotide to 13-glucosyl-5-hydroxymethylcytosine, which prevents oxidation. The TET2 enzyme causes oxidation of 5-methylcytosine in the target polynucleotide to 5-hydroxymethylcytosine, which in turn is converted to 13-glucosyl-5-hydroxymethylcytosine by the T4 bacteriophage 13-glucosyltransferase. The TET2 enzyme also causes oxidation of 5-formylcytosine in the target polynucleotide to 5-carboxylcytosine. Subsequent treatment with APOBEC3A converts unmodified cytosines in the target polynucleotide to uracil, as well as 5-carboxylcytosine (including residues that used to be 5-formylcytosine) to a limited extent. Accordingly, EM-seq allows identification of the modified cytosines 5-mC and 5-hmC (as protected glycosyl residues) by reading them as C; whereas unmodified C is converted to U: 5fC and 5-caC are converted to nucleobases which are read as T/U to a limited extent.

### Modified APOBEC

Modified APOBEC sequencing involves using a mutant APOBEC3A enzyme as the conversion agent, which is described in more detail in the Reference Examples 1 to 4 below. This process is described in U.S. Provisional Application 63/328,444, which is incorporated herein by reference.

### TAPS

TET-assisted pyridine borane sequencing (TAPS) involves using a TET1 enzyme as the further agent and pyridine borane as the conversion agent. This process is described in Liu et al. (Nature Biotechnology, 2019, 37, pp. 424-429), which is incorporated herein by reference. The TET1 enzyme causes oxidation of 5-methylcytosine, 5-hydroxymethylcytosine and 5-formylcytosine in the target polynucleotide to 5-carboxylcytosine. Subsequent treatment with pyridine borane converts 5-carboxylcytosine (including residues that used to be 5-methylcytosine, 5-hydroxymethylcytosine and 5-formylcytosine) to dihydrouracil, but does not convert unmodified cytosine. Accordingly, TAPS allows identification of the modified cytosines 5-mC, 5-hmC, 5-fC and 5-caC by reading them as T/U; whereas unmodified cytosine is read as C.

### TAPS/3

TET-assisted pyridine borane sequencing with 13-glucosyltransferase blocking (TAPSI3) involves using a T413-glucosyltransferase and a TET1 enzyme as the further agents, and pyridine borane as the conversion agent. This process is described in Liu et al. (Nature Communications, 2021, 12,618), which is incorporated herein by reference. The T413-glucosyltransferase converts 5-hydroxymethylcytosine in the target polynucleotide to 13-glucosyl-5-hydroxymethylcytosine, which prevents oxidation. The TET1 enzyme causes oxidation of 5-methylcytosine and 5-formylcytosine in the target polynucleotide to 5-carboxylcytosine. Subsequent treatment with pyridine borane converts 5-carboxylcytosine (including residues that used to be 5-methylcytosine and 5-formylcytosine) to dihydrouracil, but does not convert unmodified cytosine or 13-glucosyl-5-hydroxymethylcytosine. Accordingly, TAPSI3 allows identification of the modified cytosines 5-mC, 5-fC and 5-caC by reading them as T/U; whereas unmodified cytosine and 5-hmC are read as C.

### CAPS

Chemical-assisted pyridine borane sequencing (CAPS) involves using a potassium ruthenate (K2RuQ4) as the further agent and 2-picoline borane as the conversion agent. This process is described in Liu et al. (Nature Communications, 2021. 12,618), which is incorporated herein by reference. Potassium ruthenate causes oxidation of 5-hydroxymethylcytosine in the target polynucleotide to 5-formylcytosine. Subsequent treatment with 2-picoline borane converts 5-formylcytosine (including residues that used to be 5-hydroxymethylcytosine) and 5-carboxylcytosine to dihydrouracil, but does not convert unmodified cytosine or 5-methylcytosine. Accordingly, CAPS allows identification of the modified cytosines 5-hmC, 5-fC and 5-caC by reading them as T/U; whereas unmodified cytosine and 5-mC are read as C.

### PS

Pyridine borane sequencing (PS) involves using pyridine borane as the conversion agent. This process is described in Liu et al. (Nature Communications, 2021, 12, 618), which is incorporated herein by reference. Treatment with pyridine borane converts 5-formylcytosine and 5-carboxylcytosine to dihydrouracil, but does not convert unmodified cytosine, 5-methylcytosine or 5-hydroxymethylcytosine. Accordingly, PS allows identification of the modified cytosines 5-fC and 5-caC by reading them as T/U; whereas unmodified cytosine, 5-mC and 5-hmC are read as C.

### PS-c

Pyridine borane sequencing for 5-caC (PS-c) involves using O-ethylhydroxylamine as the further agent and pyridine borane as the conversion agent. This process is described in Liu et al. (Nature Communications, 2021, 12, 618), which is incorporated herein by reference. The O-ethylhydroxylamine converts 5-formylcytosine to an oxime derivative, which prevents 5-formylcytosine from converting to dihydrouracil. Subsequent treatment with pyridine borane converts 5-carboxylcytosine to dihydrouracil, but does not convert unmodified cytosine, 5-methylcytosine, 5-hydroxycytosine or the oxime derivative of 5-formylcytosine. Accordingly, PS-callows identification of the modified cytosine 5-caC by reading it as T/U; whereas unmodified cytosine, 5-mC, 5-hmC and 5-fC are read as C.

### Genetically Unrelated Polynucleotides

In some embodiments, the library may be prepared using PCR stitching methods, such as (splicing by) overlap extension PCR (also known as OE-PCR or SOE-PCR), as described in more detail in e.g. Higuchi et al. (Nucleic Acids Res., 1988, vol. 16, pp. 7351-7367), which is incorporated herein by reference. This procedure may be used, for example, for preparing templates including concatenated polynucleotide sequences comprising a first portion and a second portion, wherein the first portion and the second portion are different polynucleotide sequences (e.g. genetically unrelated, and/or obtained from different sources). A representative process for conducting PCR stitching for a human and PhiX library is shown in FIG. 35.

As used herein, the term “genetically unrelated” refers to portions which are not related in the sense of being any two of the group consisting of: forward strands, reverse strands, forward complement strands, and reverse complement strands. However, the “genetically unrelated” sequences could be different fragment sequences which are derived from the same source, but are different fragments from that source (e.g. from the same fragmented library preparation process). This includes sequences that can be overlapping in sequence (but not identical in sequence).

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures. e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originated in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydroshear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3-hydroxyl. Standard protocols, e.g., protocols for sequencing using, for example, the Illumina platform, instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Computing Systems

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Peri, Matlab, SAS, SPSS, Python, Ruby, Pascal, Delphi, Rand PHP. In some embodiments, the methods are written in C, C#, C++, Fortran, Java, Peri, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Peri interfaces to submit, monitor and manage large sequence analysis jobs on a cluster in serial or parallel configurations.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “cluster” or “clump” refers to a group of molecules. e.g., a group of DNA, or a group of signals. In some embodiments, the signals of a cluster are derived from different features. In some embodiments, a signal clump represents a physical region covered by one amplified oligonucleotide. In various examples, a physical region may be a tile, a sub-tile, a lane or a sub-lane on a flow cell, etc. Each signal clump could be ideally observed as several signals. Accordingly, duplicate signals could be detected from the same clump of signals. In some embodiments, a cluster or clump of signals can comprise one or more signals or spots that correspond to a particular feature. When used in connection with microarray devices or other molecular analytical devices, a cluster can comprise one or more signals that together occupy the physical region occupied by an amplified oligonucleotide (or other polynucleotide or polypeptide with a same or similar sequence). For example, where a feature is an amplified oligonucleotide, a cluster can be the physical region covered by one amplified oligonucleotide. In other embodiments, a cluster or clump of signals need not strictly correspond to a feature. For example, spurious noise signals may be included in a signal cluster but not necessarily be within the feature area. For example, a cluster of signals from four cycles of a sequencing reaction could comprise at least four signals.

As used herein, a “flow cell” can include a device having a lid extending over a reaction structure to form a flow channel therebetween that is in communication with a plurality of reaction sites of the reaction structure, and can include a detection device that is configured to detect designated reactions that occur at or proximate to the reaction sites.

A flow cell may include a solid-state light detection or “imaging” device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDs)). The excitation light may have a predetermined wavelength or wavelengths, including a range of wavelengths. The fluorescent labels excited by the incident excitation light may provide emission signals (e.g., light of a wavelength or wavelengths that differ from the excitation light and, potentially, each other) that may be detected by the light sensors of the flow cell.

Flow cells described herein may be configured to perform various biological or chemical processes. More specifically, the flow cells described herein may be used in various processes and systems where it is desired to detect an event, property, quality, or characteristic that is indicative of a designated reaction. For example, flow cells described herein may include or be integrated with light detection devices, biosensors, and their components, as well as bioassay systems that operate with biosensors. The flow cells may be configured to facilitate a plurality of designated reactions that may be detected individually or collectively. The flow cells may be configured to perform numerous cycles in which the plurality of designated reactions occurs in parallel. For example, the flow cells may be used to sequence a dense array of DNA features through iterative cycles of enzymatic manipulation and light or image detection/acquisition. As such, the flow cells may be in fluidic communication with one or more microfluidic channels that deliver reagents or other reaction components in a reaction solution to a reaction site of the flow cells. The reaction sites may be provided or spaced apart in a predetermined manner, such as in a uniform or repeating pattern. Alternatively, the reaction sites may be randomly distributed. Each of the reaction sites may be associated with one or more light guides and one or more light sensors that detect light from the associated reaction site. In one example, light guides include one or more filters for filtering certain wavelengths of light. The light guides may be, for example, an absorption filter (e.g., an organic absorption filter) such that the filter material absorbs a certain wavelength (or range of wavelengths) and allows at least one predetermined wavelength (or range of wavelengths) to pass therethrough. In some flow cells, the reaction sites may be located in reaction recesses or chambers, which may at least partially compartmentalize the designated reactions therein.

As used herein, the term “spot radius” or “cluster radius” refers to a defined radius which encompasses a diffraction-limited spot or a cluster of signals. Accordingly, by defining a cluster radius as larger or smaller, a greater number of signals can fall within the radius for subsequent ordering and selection. A cluster radius can be defined by any distance measure, such as pixels, meters, millimeters, or any other useful measure of distance.

As used herein, a “signal” refers to a detectable event such as an emission, such as light emission, for example, in an image. Thus, in some embodiments, a signal can represent any detectable light emission that is captured in an image (i.e., a “spot”). Thus, as used herein, “signal” can refer to an actual emission from a feature of the specimen, or can refer to a spurious emission that does not correlate to an actual feature. Thus, a signal could arise from noise and could be later discarded as not representative of an actual feature of a specimen.

As used herein, an “intensity” of an emitted light refers to the intensity of the light transferred per unit area, where the area is measured on the plane perpendicular to the direction of propagation of the light ray, and where the intensity is the amount of energy transferred per unit time. In some embodiments, signal “strength”, “amplitude”, “magnitude” or “level” may be used synonymously with signal intensity. In some embodiments, an image taken by a detector is approximately or proportional to an intensity map integrated over some amount of time. In some embodiments, the signal of a diffraction-limited spot of a DNA cluster is extracted from the image as the total intensity included in the spot, up to a factor of the integration time. For example, the signal of a DNA cluster may be defined as the intensity included within the spot radius of the DNA cluster, up to a factor of the integration time. In other embodiments, the peak intensity value found within the spot radius may be used to represent the signal of the DNA cluster, up to a factor of the integration time.

As used herein, the process of aligning the template of signal positions onto a given image is referred to as “registration”, and the process for determining an intensity value or an amplitude value for each signal in the template for a given image is referred to as “intensity extraction”. For registration, the methods and systems provided herein may take advantage of the random nature of signal clump positions by using image correlation to align the template to the image.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof.

The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The polymerase used is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Kienow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, Tfil DNA Polymerase, Tth DNA Polymerase, RepliPHI Phi29 Polymerase. Tli DNA polymerase, eukaryotic DNA polymerase beta, telomerase, Therminator™ polymerase (New England Biolabs), KOO HiFim DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants. “Encode” or “parse” are verbs referring to transferring from one format to another, and refers to transferring the genetic information of target template base sequence into an arrangement of reporters.

Nucleosides and nucleotides may be labeled at sites on the sugar or nucleobase. A dye may be attached to any position on the nucleotide base, for example, through a linker. In particular embodiments, Watson-Crick base pairing can still be carried out for the resulting analog. Particular nucleobase labeling sites include the CS position of a pyrimidine base or the C7 position of a 7-deaza purine base. A linker group may be used to covalently attach a dye to the nucleoside or nucleotide. As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

Various different types of linkers having different lengths and chemical properties can be used. The term “linker” encompasses any moiety that is useful to connect one or more molecules or compounds to each other, to other components of a reaction mixture, and/or to a reaction site. For example, a linker can attach a reporter molecule or “label” (e.g., a fluorescent dye) to a reaction component. In certain embodiments, the linker is a member selected from substituted or unsubstituted alkyl (e.g., a 2-5 carbon chain), substituted or unsubstituted heteroalkyl, substituted or unsubstituted aryl, substituted or unsubstituted heteroaryl, substituted or unsubstituted cycloalkyl, and substituted or unsubstituted heterocycloalkyl. In one example, the linker moiety is selected from straight- and branched carbon-chains, optionally including at least one heteroatom (e.g., at least one functional group, such as ether, thioether, amide, sulfonamide, carbonate, carbamate, urea and thiourea), and optionally including at least one aromatic, heteroaromatic or non-aromatic ring structure (e.g., cycloalkyl, phenyl). In certain embodiments, molecules that have trifunctional linkage capability are used, including, but are not limited to, cynuric chloride, mealamine, diaminopropanoic acid, aspartic acid, cysteine, glutamic acid, pyroglutamic acid, S-acetylmercaptosuccinic anhydride, carbobenzoxylysine, histine, lysine, serine, homoserine, tyrosine, piperidinyl-1,1-amino carboxylic acid, diaminobenzoic acid, etc. In certain specific embodiments, a hydrophilic PEG (polyethylene glycol) linker is used.

In certain embodiments, linkers are derived from molecules which comprise at least two reactive functional groups (e.g., one on each terminus), and these reactive functional groups can react with complementary reactive functional groups on the various reaction components or used to immobilize one or more reaction components at the reaction site. “Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitre, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like.

Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

In some embodiments, one or more dye or label molecules may attach to the nucleotide base by non-covalent interactions, or by a combination of covalent and non-covalent interactions via a plurality of intermediating molecules. In one example, a nucleotide or a nucleotide analog, being newly incorporated by the polymerase synthesizing from a target polynucleotide, is initially unlabeled. Then, one or more fluorescent labels may be introduced to the nucleotide or nucleotide analog by binding to labeled affinity reagents containing one or more fluorescent dyes. Uses of unlabeled nucleotides and affinity reagents in sequencing by synthesis have been disclosed in U.S. Publication No. 2013/0079232, which is incorporated herein by reference. For example, one, two, three or each of the four different types of nucleotides (e.g., dATP, dCTP, dGTP and dTTP or dUTP) in the reaction mix may be initially unlabeled. Each of the four types of nucleotides (e.g., dNTPs) may have a 3′ hydroxy blocking group to ensure that only a single base can be added by a polymerase to the 3′ end of a copy polynucleotide being synthesized from the target polynucleotide. After incorporation of an unlabeled nucleotide, an affinity reagent may be then introduced that specifically binds to the incorporated dNTP to provide a labeled extension product comprising the incorporated dNTP. The affinity reagent may be designed to specifically bind to the incorporated dNTP via antibody-antigen interaction or ligand-receptor interaction, for example. The dNTP may be modified to include a specific antigen, which will pair with a specific antibody included in the corresponding affinity reagent. Thus, one, two, three or each of the four different types of nucleotides may be specifically labeled via their corresponding affinity reagents. In some embodiments, the affinity reagents may include small molecules or protein tags that may bind to a hapten moiety of the nucleotide (such as streptavidin-biotin, anti-DIG and DIG, anti-DNP and DNP), antibody (including but not limited to binding fragments of antibodies, single chain antibodies, bispecific antibodies, and the like), aptamers, knottins, affimers, or any other known agent that binds an incorporated nucleotide with a suitable specificity and affinity. In some embodiments, the hapten moiety of the unlabeled nucleotide may be attached to the nucleobase through a cleavable linker, which may be cleaved under the same reaction condition as that for removing the 3 blocking group. In some embodiments, one affinity reagent may be labeled with multiple copies of the same fluorescent dye, for example, 1, 2, 3, 4, 5, 6, 8, 10, 12, 15 copies of the same dye. In some embodiments, each affinity reagent may be labeled with a different number of copies of the same fluorescent dye. In some embodiments, a first affinity reagent may be labeled with a first number of a first fluorescent dye, a second affinity reagent may be labeled with a second number of a second fluorescent dye, a third affinity reagent may be labeled with a third number of a third fluorescent dye, and a fourth affinity reagent may be labeled with a fourth number of a fourth fluorescent dye. In some embodiments, each affinity reagent may be labeled with a distinct combination of one of more types of dye, where each type of dye has a certain copy number. In some embodiments, different affinity reagents may be labeled with different dyes that can be excited by the same light source, but each dye will have a distinguishable fluorescent intensity or a distinguishable emission spectrum. In some embodiments, different affinity reagents may be labeled with the same dye in different molar ratios to create measurable differences in their fluorescent intensities.

A nucleotide analog may be attached to or associated with one or more photo-detectable labels to provide a detectable signal. In some embodiments, a photo-detectable label may be a fluorescent compound, such as a small molecule fluorescent label. Fluorescent molecules (fluorophores) suitable as a fluorescent label include, but are not limited to: 1,5 IAEDANS; 1,8-ANS; 4-methylumbelliferone; 5-carboxy-2,7-dichlorofluorescein; 5-carboxyfluorescein (5-FAM); fluorescein amidite (FAM); 5-carboxynapthofluorescein; tetrachloro-8-carboxyfluorescein (TET); hexachloro-6-carboxyfluorescein (HEX); 2,7-dimethoxy-4,5-dichloro-6-carboxyfluorescein (JOE); VIC®; NED™; tetramethylrhodamine (TMR); 5-carboxytetramethylrhodamine (5-TAMRA); 5-HAT (Hydroxy Tryptamine); 5-hydroxy tryptamine (HAT); 5-ROX (carboxy-X-rhodamine); 6-carboxyrhodamine 6G; 6-JOE; Light Cycler® red 610; Light Cycler® red 640; Light Cycler® red 670; Light Cycler® red 705; 7-amino-4-methylcoumarin; 7-aminoactinomycin D (7-AAD); 7-hydroxy-4-methylcoumarin; 9-amino-6-chloro-2-methoxyacridine; 6-methoxy-N-(4-aminoalkyl)quinolinium bromide hydrochloride (ABQ); Acid Fuchsin; ACMA (9-amino-8-chloro-2-methoxyacridine); Acridine Orange; Acridine Red; Acridine Yellow; Acriflavin; Acriflavin Feulgen SITSA; AFPs-AutoFluorescent Protein-(Quantum Biotechnologies); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (CalcofluorWhite); TMR: TO-PRO-1; TO-PRO-3; TO-PRO-5; TOTO-1; TOTO-3; TriColor (PE-Cy5); TRITC (TetramethylRodamine-IsoThioCyanate); True Blue; TruRed; Ultralite; Uranine B; Uvitex SFC; WW 781; X-Rhodamine; X-Rhodamine-5-(and-6)-Isothiocyanate (5(6)-XRITC); Xylene Orange; Y66F; Y66H; Y66 W; YO-PRO-1; YO-PRO-3; YOYO-1; interchelating dyes such as YOYO-3, Sybr Green, Thiazole orange; members of the Alexa Fluor® dye series (from Molecular Probes/Invitrogen) which cover a broad spectrum and match the principal output wavelengths of common excitation sources such as Alexa Fluor 350, Alexa Fluor 405, 430, 488, 500, 514, 532, 546, 555, 568, 594, 610, 633, 635, 647, 660, 680, 700, and 750; members of the Cy Dye fluorophore series (GE Healthcare), also covering a wide spectrum such as Cy3, Cy3B, Cy3.5, Cy5, Cy5.5, Cy?; members of the Oyster® dye fluorophores (Denovo Biolabels) such as Oyster-500, -550, -556, 645, 650, 656; members of the DY-Labels series (Dyomics), for example, with maxima of absorption that range from 418 nm (DY-415) to 844 nm (DY-831) such as DY-415, -495, -505, -547, -548, -549, -550, -554, -555, -556, -560, -590, -610, -615, -630, -631, -632, -633, -634, -635, -836, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL; members of the ATTO series of fluorescent labels (ATTO-TEC GmbH) such as ATTO 390, 425, 465, 488, 495, 520, 532, 550, 565, 590, 594, 610, 611X, 620, 633, 635, 637, 647, 647N, 655, 680, 700, 725, 740: members of the CAL Fluor® series or Quasar® series of dyes (Biosearch Technologies) such as CAL FluorO Gold 540, CAL Fluor® Orange 560. Quasar® 570, CAL Fluor® Red 590, CAL Fluor® Red 610, CAL Fluor Red 635, Quasar®570, and Quasar® 670. In some embodiments, a first photo-detectable label interacts with a second photo-detectable moiety to modify the detectable signal, e.g., via fluorescence resonance energy transfer (“FRET”; also known as Forster resonance energy transfer).

The fluorescent labels utilized by the systems and methods disclosed herein can have different peak absorption wavelengths, for example, ranging from 400 nm to 800 nm. In some embodiments, the peak absorption wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak absorption wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different peak emission wavelength, for example, ranging from 400 nm to 800 nm. In some embodiments, the peak emission wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak emission wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different Stokes shift, for example, ranging from 10 nm to 200 nm. In some embodiments, the stoke shift can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the stoke shift can be at least, or at most, 10, 20, 30, 40, 50, 60, 70, 80, 90, 100, 110, 120, 130, 140, 150, 160, 170, 180, 190, or 200 nm.

In some embodiments, the distance between the peak emission wavelengths of any two fluorescent labels can vary, for example, ranging from 10 nm to 200 nm. In some embodiments, the distance between the peak emission wavelengths of any two fluorescent labels can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the distance between the peak emission wavelengths of any two fluorescent labels can be at least, or at most, 10, 20, 30, 40, 50, 60, 70, 80, 90, 100, 110, 120, 130, 140, 150, 160, 170, 180, 190, or 200 nm.

A “light source” may be any device capable of emitting energy along the electromagnetic spectrum. A light source may be a source of visible light (VIS), ultraviolet light (UV) and/or infrared light (IR). “Visible light” (VIS) generally refers to the band of electro-magnetic radiation with a wavelength from about 400 nm to about 750 nm. “Ultraviolet (UV) light” generally refers to electromagnetic radiation with a wavelength shorter than that of visible light, or from about 10 nm to about 400 nm range. “Infrared light” or infrared radiation (IR) generally refers to electromagnetic radiation with a wavelength greater than the VIS range, or from about 750 nm to about 50,000 nm. A light source may also provide full spectrum light. Light sources may output light from a selected wavelength or a range of wavelengths. In some embodiments of the invention, the light source may be configured to provide light above or below a predetermined wavelength, or may provide light within a predetermined range. A light source may be used in combination with a filter, to selectively transmit or block light of a selected wavelength from the light source. A light source may be connected to a power source by one or more electrical connectors; an array of light sources may be connected to a power source in series or in parallel. A power source may be a battery, or a vehicle electrical system or a building electrical system. The light source may be connected to a power source via control electronics (control circuit); control electronics may comprise one or more switches. The one or more switches may be automated, or controlled by a sensor, timer or other input, or may be controlled by a user, or a combination thereof. For example, a user may operate a switch to turn on a UV light source; the light source may be applied on a constant basis until it is turned off, or it may be pulsed (repeated on/off cycles) until it is turned off. In some embodiments, the light source may be switched from a continuously-on state to a pulsed state, or vice versa. In some embodiments, the light source may be configured to be brightening or darkening overtime.

For operation, the light source may be connected to a power source capable of providing sufficient intensity to illuminate the sample. Control electronics may be used to switch the intensity on or off based on input from a user or some other input, and can also be used to modulate the intensity to a suitable level (e.g. to control brightness of the output light). Control electronics may be configured to turn the light source on and off as desired. Control electronics may include a switch for manual, automatic, or semi-automatic operation of the light sources. The one or more switches may be, for example, a transistor, a relay or an electromechanical switch. In some embodiments, the control circuit may further comprise an AC-DC and/or a DC-DC converter for converting the voltage from the voltage source to an appropriate voltage for the light source. The control circuit may comprise a DC-DC regulator for regulation of the voltage. The control circuit may further comprise a timer and/or other circuitry elements for applying electric voltage to the optical filter for a fixed period of time following the receipt of input. A switch may be activated manually or automatically in response to predetermined conditions, or with a timer. For example, control electronics may process information such as user input, stored instructions, or the like.

One or more of a plurality of light sources may be provided. In some embodiments, each of the plurality of light sources may be the same. Alternatively, one or more of the light sources may vary. The light characteristics of the light emitted by the light sources may be the same or may vary. A plurality of light sources may or may not be independently controllable. One or more characteristic of the light source may or may not be controlled, including but not limited to whether the light source is on or off, brightness of light source, wavelength of light, intensity of light, angle of illumination, position of light source, or any combination thereof.

In some embodiments, light output from a light source may be from about 350 to about 750 nm, or any amount or range therebetween, for example from about 350 nm to about 360, 370, 380, 390, 400, 410, 420, 430 or about 450 nm, or any amount or range therebetween. In other embodiments, light from a light source may be from about 550 to about 700 nm, or any amount or range therebetween, for example from about 550 to about 560, 570, 580, 590, 600, 610, 620, 630, 640, 650, 660, 670, 680, 690 or about 700 nm, or any amount or range therebetween. In some embodiments, the wavelength of the light generated by the light source can vary, for example, ranging from 400 nm to 800 nm. In some embodiments, the wavelength of the light generated by the light source can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 780, 770, 780, 790, 800 nm, or a number or a range between any two of these values. In some embodiments, the wavelength of the light generated by the light source can be at least, or at most, 400, 410, 420, 430, 440, 450, 480, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm. The light source may be capable of emitting electromagnetic waves in any spectrum. In some embodiments, the light source may have a wavelength falling between 10 nm and 100 μm. In some embodiments, the wavelength of light may fall between 100 nm to 5000 nm, 300 nm to 1000 nm, or 400 nm to 800 nm. In some embodiments, the wavelength of light may be less than, and/or equal to 10 nm, 100 nm, 200 nm, 300 nm, 400 nm, 500 nm, 600 nm, 700 nm, 800 nm, 900 nm, 1000 nm, 1100 nm, 1200 nm, 1300 nm, 1500 nm, 1750 nm, 2000 nm, 2500 nm, 3000 nm, 4000 nm, or 5000 nm.

In one example, a light source may be a light-emitting diode (LED) (e.g., gallium arsenide (GaAs) LED, aluminum gallium arsenide (AlGaAs) LED, gallium arsenide phosphide (GaAsP) LED, aluminum gallium indium phosphide (AlGaInP) LED, gallium(II) phosphide (GaP) LED, indium gallium nitride (InGaN)/gallium(III) nitride (GaN) LED, or aluminum gallium phosphide (AlGaP) LED). In another example, a light source can be a laser, for example a vertical cavity surface emitting laser (VCSEL) or other suitable light emitter such as an Indium-Gallium-Aluminum-Phosphide (InGaAlP) laser, a Gallium-Arsenic Phosphide/Gallium Phosphide (GaAsP/GaP) laser, or a Gallium-Aluminum-Arsenide/Gallium-Aluminum-Arsenide (GaAlAs/GaAs) laser. Other examples of light sources may include but are not limited to electron stimulated light sources (e.g., Cathodoluminescence, Electron Stimulated Luminescence (ESL light bulbs), Cathode ray tube (CRT monitor), Nixie tube), incandescent light sources (e.g., Carbon button lamp, Conventional incandescent light bulbs, Halogen lamps, Globar, Nemst lamp), electroluminescent (EL) light sources (e.g., Light-emitting diodes-Organic light-emitting diodes, Polymer light-emitting diodes, Solid-state lighting, LED lamp, Electroluminescent sheets Electroluminescent wires), gas discharge light sources (e.g., Fluorescent lamps, Inductive lighting, Hollow cathode lamp, Neon and argon lamps, Plasma lamps, Xenon flash lamps), or high-intensity discharge light sources (e.g., Carbon arc lamps, Ceramic discharge metal halide lamps, Hydrargyrum medium-arc iodide lamps, Mercury-vapor lamps, Metal halide lamps, Sodium vapor lamps, Xenon arc lamps). Alternatively, a light source may be a bioluminescent, chemiluminescent, phosphorescent, or fluorescent light source.

As used herein, an “optical channel” is a predefined profile of optical frequencies (or equivalently, wavelengths). For example, a first optical channel may have wavelengths of 500 nm-800 nm. To take an image in the first optical channel, one may use a detector which is only responsive to 500 nm-600 nm light, or use a bandpass filter having a transmission window of 500 nm-600 nm to filter the incoming light onto a detector responsive to 300 nm-800 nm light. A second optical channel may have wavelengths of 300 nm-450 nm and 850 nm-900 nm. To take an image in the second optical channel, one may use a detector responsive to 300 nm-450 nm light and another detector responsive to 850 nm-900 nm light and then combine the detected signals of the two detectors. Alternatively, to take an image in the second optical channel, one may use a bandstop filter which rejects 451 nm-849 nm light in front of a detector responsive to 300 nm-900 nm light.

### ADDITIONAL NOTES

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. The term “partially” is used to indicate that an effect is only in part or to a limited extent.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### EXAMPLES

### Example 1—Methylation Analysis on Methylated pUC19 Sample Using 9 QaM

### Oligo Sequences:

For transposon annealing (underline indicates ME′ or ME):

ME′-HYB2
(SEQ ID NO. 21)
/5Phos/CTGTCTCTTATACACATCTGAGTAAGTGGAAGAGATAGGAAGG
ME′-HYB2′
(SEQ ID NO. 22)
/5Phos/CTGTCTCTTATACACATCTCCTTCCTATCTCTTCCACTTACTC
Biotin-A14-ME
(SEQ ID NO. 9)
Biotin-TCGTCGGCAGCGTCAGATGTGTATAAGAGACAG
Biotin-815-ME
(SEQ ID NO. 10)
Biotin-GTCTCGTGGGCTCGGAGATGTGTATAAGAGACAG

### Sequencing Oligos (Underline Indicates ME):

HYB2-ME
(SEQ ID NO. 12)
GAGTAAGTGGAAGAGATAGGAAGGAGATGTGTATAAGAGACAG
HYB2′-ME
(SEQ ID NO. 14)
CCTTCCTATCTCTTCCACTTACTCAGATGTGTATAAGAGACAG

### Preparation of Forked Adaptors:

1. 5 μ1 of 200 μM stock of biotin-A 14-ME oligo was combined with 10 μl of 100 μM stock of ME′-HYB2 oligo. 2 μ1 of 10×TEN Annealing buffer (Illumina) and 3 μ1 of IDTE buffer (Illumina) was added (“A14” transposome mixture).
2. Separately, 5 μ1 of 200 μM stock of biotin-B15-ME oligo was combined with 10 μ1 of 100 μM stock of ME′-HYB2′ oligo. 2 μ1 of 10×TEN Annealing buffer (Illumina) and 3 μ1 of IDTE buffer (Illumina) was added (“B15” transposome mixture) with 10×TEN and IDTE buffers.
3. Each mixture was heated to 95 C for 30 s followed by a slow cool (0.1 C/s ramp rate) to 10 C.
4. 2 μ1 of each annealed mixture was combined with 46 μ1 of Standard Storage Buffer (contains 50% glycerol, Illumina) and 2 μ1 of Tn5 transposase (˜90 μM stock).
5. Each mixture was mixed and incubated overnight at 37 C. Following the incubation step, the two separately prepared transposome complexes were combined together by adding 50 μ1 of each to another 100 μ1 of Standard Storage Buffer to give 200 μ1 of 1 μM transposome mix.
   Loading of Forked Adaptors onto Beads:
1. 200 μ1 of MyOne T1 Streptavidin beads (Thermofisher) were washed twice with 200 μ1 Tagmentation Wash buffer (TWB, Illumina).
2. Beads were resuspended in 960 μ1 of TWB and 40 μ1 of 1 μM transposome mix from step 5 of “Preparation of forked adaptors” was added.
3. Beads were mixed on a rotator for 30 mins to 1 hr at room temperature.
4. Beads were put on a magnet and beads were washed twice with TWB.
5. Beads were resuspended in original volume (200 μ1) of BLT Storage Buffer (Illumina). The BLTs were stored at 4 C until needed.

### Tagmentation:

1. 10 μl BLT (bead linked transposomes) from step 5 of “Loading of forked adaptors onto beads” were combined with 100 ng DNA in 30 μ1 (pUC19 methylated control DNA) and 10 μ1 of TB1 (Sx Tag buffer, Illumina).
2. The combination was mixed and incubated at SSC for Smin, followed by a hold step at 10 C.
3. 10 μl ST2 Stop buffer was added and mixed.
4. The mixture was incubated at room temp for 5 mins.
5. The tubes were transferred to a magnet.
6. The beads were washed twice with 100 μ1 Tagmentation Wash buffer (TVB, Illumina).
7. The beads were resuspended in 50 μ1 of ELM (Extension Ligation Mix, Illumina).
8. The mixture was incubated at 37 C for 5 mins, then SOC for 5 mins, followed by a hold step at 10 C.

### Hybridisation and Extension on Beads:

1. The tubes from step 8 of “Tagmentation” were placed on a magnet until the BLT beads pelleted.
2. The beads were washed once with 200 μ1 of Tagmentation Wash Buffer (TWB, Illumina).
3. The beads were washed once with 200 μ1 of 0.1N NaOH—the beads were left to sit in 0.1N NaOH for 30 s during this wash step.
4. Beads were washed once with 200 μ1 of TWB.
5. Bears were resuspended in 100 μ1 of HT1 (Hybridisation Buffer, Illumina).
6. Beads were heated in HT1 to 70 C for 30 s followed by a slow cool (0.1 C/s) down to 10 C.
7. Beads were washed twice with 200 μ1 of TWB.
8. Beads were resuspended in 100 μ1 of PAM (Patterned Amplification Mix, Illumina) supplemented with 50 mM KCl.
9. Beads were heated in PAM to SOC for 5 mins, then 60 C for 5 mins.
10. Beads were washed twice with 200 μ1 of TWB.
11. Beads were resuspended in 50 μ1 of RSB (Resuspension Buffer, Illumina).

### Methylation Analysis Conversion Method:

1. The following TET master mix (TET MM) was prepared and kept on ice:

| | 1x (μl) | 4.5x (μl) |
|---|---|---|
| Water | 9.00 | 40.50 |
| Reconstituted TET2 Reaction Buffer | 10 | 45 |
| (NEB EM-seq kit) | | |
| Oxidation Supplement (NEB EM-seq kit) | 1 | 4.5 |
| DTT (NEB EM-seq kit) | 1 | 4.5 |
| TET2 (NEB EM-seq kit) | 4 | 18 |
| Total | 25 | 112.5 |

2. On ice, 25 μ1 of TET MM was added to 20 μ1 of adaptor-ligated DNA in the form of BLTs in RSB (from step 11 of “Hybridisation and extension on beads”).
3. The mixture was vortexed and centrifuged briefly.
4. 500 mM of Fe(II) solution (NEB EM-seq kit) was freshly prepared and diluted by adding 1 μl to 1249 μ1 of water.
5. 5 μ1 of the diluted Fe(II) solution was added to the 45 μl of adaptor-ligated DNA with TET MM prepared in step 2.
6. The mixture was vortexed (or pipette mixed 10×), centrifuged briefly, incubated for 1 hr at 37 C, then put on ice.
7. 1 μl of Stop reagent was added, vortexed (or pipette mixed 10×), and incubated at 37 C for 30 mins.
8. The beads were washed once with 100 μ1 Wash buffer, and then resuspended in 35 μ1 water.
9. In a PCR tube, the 35 μ1 of TET-oxidised DNA from step 8 was combined with
10 μl of sodium acetate/acetic acid buffer (pH 4.3) and 5 μl of 1 M pyridine borane. The mixture was incubated overnight at 40 C.
10. The beads were washed twice with 100 μ1 Wash buffer, then resuspended in 20 μl of RSB.
11. The 20 ul of beads+DNA in RSB from step 10 was combined with 25 μ1 of QSU Mastermix (NEB) and 5 μ1 of UDI primers (Unique Dual Index primers. Illumina).
12. The mixture was amplified by PCR: cycling procedure—98 C for 30 s followed by 3 cycles of (98 C 10 s, 62 C 30 s, 65 C 3 min), then 6 cycles of (98 C 10 s, 62 C 30 s, 65 C 30 s), 65 C for 5 mins and then hold at 4 C.
13. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq. Standard clustering on the MiniSeq and a standard first hyb was conducted for the 1st 36 cycles of sequencing.

A custom second hyb was used from the “Cust3” position of the reagent cartridge. This primer hyb maintains a higher temperature (60 C) than normal during the post-hyb wash (which usually drops to 40 C). This higher temperature was to ensure that the right sequencing primers hyb to the right places on the cluster strands.

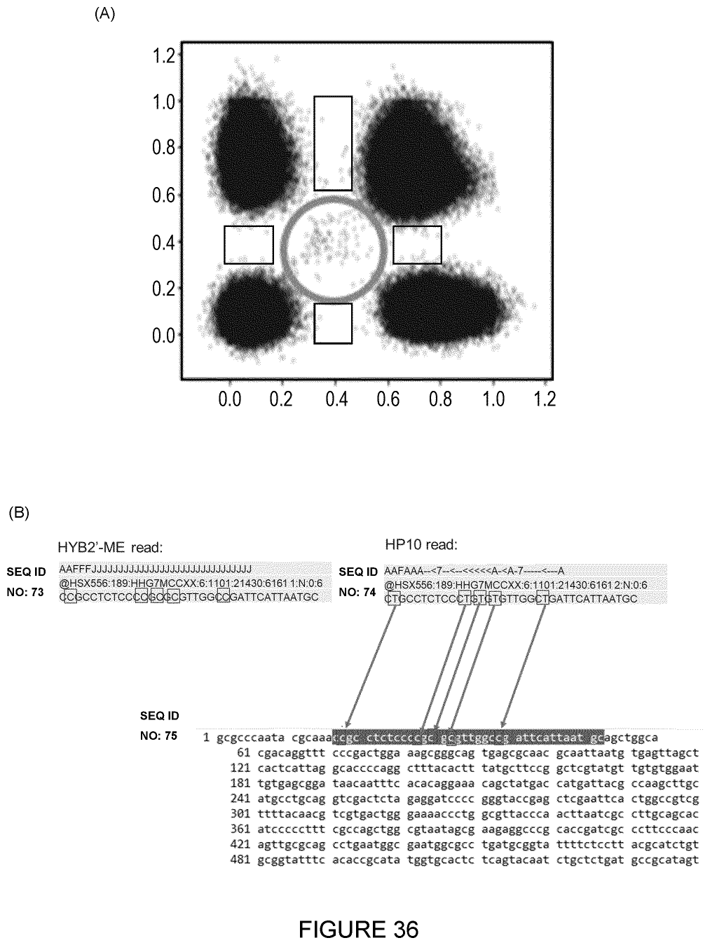

The primer mix for this custom hyb was HP10 R1 primer mix (Illumina) spiked with 0.5 μM each of HYB2′-ME and HYB2-ME primers. These primers are all unblocked and allow concurrent sequencing of both the first portion and the second portion, and so generate the 9 QaM signal during sequencing. The converted library was loaded onto the MiniSeq cartridge at 1 μM final concentration. The MiniSeq was set up to save 3 tiles of images per cycle, for later off-line analysis. The 9 QaM results are shown in FIG. 36A, where modified cytosines can be identified by a characteristic central cloud in the plot (indicated by circled region). The actual genetic sequences are shown in FIG. 36B, where modified cytosines can be assigned to cases where a C-T mismatch is observed between the HYB2′-ME read and the HP10 read.

Overall, these results (in particular the custom second hyb results) show that methylation analysis (in this case, all 5-mC, 5-hmC, 5-fC and 5-caC, as a result of TAPS analysis) can be conducted on polynucleotide sequences to identify modified cytosines. In particular, by enabling concurrent sequencing of the forward and reverse complement strands of the template (or reverse and forward complement strands of the template), modified cytosines can be identified quickly and accurately.

### Example 2—Methylation Analysis on Methylated pUC19 Sample Using 9 QaM

### Oligo Sequences:

Asterisk (*) indicates a phosphorothioate linkage.

Underline indicates 5-methylcytosine instead of cytosine (in “P5_BbvCI_P7-methylated” and “BspQI_iSce_Loop-methylated”, all cytosines are replaced with 5-methylcytosines to prevent unwanted conversion of cytosine to uracil in the adaptor sequence during bisulfite conversion).

Bold indicates nicking restriction site (or its complement) of Nt.BspQI, which recognises the following sequence (nicking site is indicated by arrow):

S′. GCTCT N″. 3′
3′. CGAGA N. 5′

[Biotin-T] indicates the following structure:

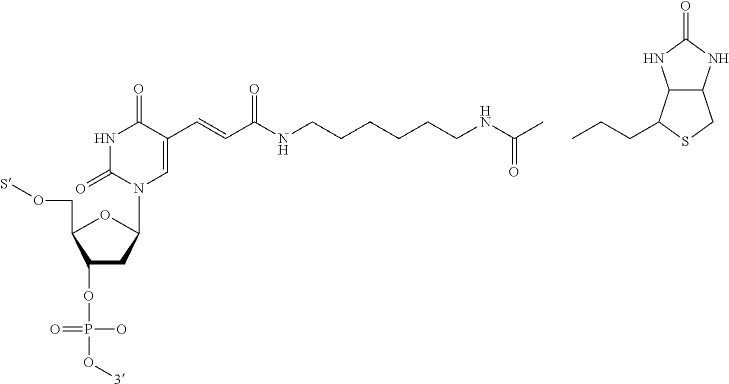

PS_BbvCI_P7 (SEQ ID NO. 69):
GCTGAGGATCTCGTATGCCGTCTTCTGCTTGUAATGATACGGCGACCAC
CGAGATCTACACTCCTCAGC*T
BspQI_iSce_Loop (SEQ ID NO. 70):
GAAGAGCACACGTCTGAACTCCAGTCACTAGGGA[Biotin-T]
AACAGGGTAATCTTTCCCTACACGACGCTCTTC*T
PS_BbvCI_P7-methylated (SEQ ID NO. 71):
GCTGAGGATCTCGTATGCCGTCTTCTGCTTGUAATGATACGGCGACCAC
CGAGATCTACACTCCTCAGC*T
BspQI_iSce_Loop-methylated (SEQ ID NO. 72):
GAAGAG A A GT TGAA TCCAGT A TAGGGA[Biotin-T]AA
AGGGTAAT TTTCCCTA CACGACGCTCTTC*T

### Adaptor Annealing:

1. A mixture of 4 μ1 of 100 μM PS_BbvCI_P7-methylated oligo, 11 μl water, 2 μ1 10×TEN buffer (Illumina) and 3 μ1 IDTE buffer was heated to 98 C for 30 s, then a slow cool to room temperature (eg. 0.1 C/s ramp down to RT). This gives a 20 μM stock of annealed P5_BbvCI_P7-methylated adaptor.
2. Separately, a mixture of 4 μ1 of 100 μM BspQI_iSce_Loop-methylated oligo, 11 μl water, 2 μ1 10×TEN buffer (Illumina) and 3 μ1 IDTE buffer was heated to 98 C for 30 s, then a slow cool to room temperature (eg. 0.1 C/s ramp down to RT). This gives a 20 μM stock of annealed BspQI_iSce_Loop-methylated adaptor.
3. Equal volumes of the 20 μM stock of annealed P5_BbvCI_P7-methylated adaptor from step 1 and 20 μM stock of annealed BspQI_iSce_Loop-methylated adaptor from step 2 are mixed together, giving a stock solution with 10 μM each of annealed P5_BbvCI_P7-methylated adaptor and annealed BspQI_iSce_Loop-methylated adaptor.

### Preparation of Library:

1. NEB Ultra II FS reagents were thawed at room temperature and kept on ice until use.
2. The Ultra II FS Enzyme mix was vortexed for 5-8 seconds prior to use and placed on ice.
3. In a 0.2 ml PCR tube on ice, 26 μ1 DNA (100 ng of input DNA (methylated pUC19 sample) diluted to 26 μ1 with Milli-Q grade water), 7 μ1 of NEBNext Ultra II FS Reaction Buffer and 2 μ1 of NEBNext Ultra II FS Enzyme Mix were added, briefly vortexed and spun in a microcentrifuge to mix.
4. In a Thermocycler with the heated lid set to 75 C, the tubes were incubated for 5 mins at 37 C, then 30 mins at 65 C then held at 4 C.
5. The following were added to the FS reaction mixture from step 4: 30 μ1 of NEBNext Ultra II Ligation Master Mix, 1 μl of NEBNext Ligation Enhancer and 2.5 μ1 of the loop adaptors P5_BbvCI_P7-methylated and BspQI_iSce_Loop-methylated (10 μM each) prepared from step 3 of “Adaptor annealing”.
6. The entire volume was pipetted up and down 10× to mix, followed by a brief spin in a microcentrifuge.
7. The mixture was incubated at 20 C for 15 mins in a thermocycler with the heated lid off.
8. 3 μ1 of USER Enzyme (NEB) was added to the ligation mix.
9. The mixture was mixed well and incubated at 37 C for 15 mins with the heated lid set to >47 C.
10. Adaptor ligated DNA was then size selected via a 0.8×SPRI (iTune beads) selection: 57 μ1 iTune beads (ILMN) were added to 68.5 μ1 of ligation reaction, mixed and incubated at RT for 5 mins.
11. The mixture was placed on a magnet for 5 mins, and the supernatant was discarded.
12. The beads were washed twice with 200 μ1 of 80% ethanol-200 μ1 80% ethanol was added with beads on the magnet, followed by a 30 s wait, and ethanol was removed, then the wash was repeated once more.
13. The last remnants of ethanol were removed with a P10 pipette and tip.
14. Beads were then air dried for 5 mins.
15. DNA was eluted from beads with 40 μ1 of 0.1×TE buffer. At this stage, 20 μ1 was saved as a “non-converted” control, the remaining 20 μ1 was treated to bisulfite conversion, following the Zymo Research EZ-96 DNA Methylation Gold MagPrep kit (steps 16-25 are taken from the instructions for this kit).
18. In a 0.2 ml PCR tube, 20 μ1 of 0.8×SPRI selected ligation and 130 μ1 of CT Conversion Reagent (comprises sodium metabisulfite) were added.
17. The mixture was incubated on a thermocycler at 98 C for 10 mins, then 64 C for 2.5 hours, followed by holding at 4 C for up to 20 hours.
18. The sample was transferred to 1.7 ml tubes for subsequent steps. 600 μ1 of M-Binding Buffer and 10 μl of MagBinding Beads were added. The mixture was vortexed for 30 s.
19. Incubate at RT for 5 mins, then place on a magnet for 5 mins.
20. The supernatant was removed and discarded. 400 μ1 of M-Wash buffer was added to the beads, and then vortexed for 30 s. The mixture was placed back on magnet until the beads pelleted.
21. The supernatant was removed and discarded.
22. 200 μ1 of M-Desulphonation Buffer was added to the beads, and then vortexed for 30 s. The mixture was incubated at RT for 15-20 mins. The mixture was then placed back on magnet until beads pelleted.
23. The supernatant was removed and discarded. 400 μ1 of M-Wash buffer was added to the beads, then vortexed for 30 s. The mixture was placed back on magnet until beads pelleted. This wash step was repeated once.
24. The supernatant after 2nd wash was removed, and the tubes were transferred to a hot block at SSC to air dry the beads for 20-30 mins and remove residual M-Wash buffer.
25. 25 μ1 of M-Elution Buffer was added to the dried beads and vortexed for 30 s. The elution mixture was heated at SSC for 4 mins then the tubes were placed back on the magnet for 1 min (or until the beads pelleted). The eluate was removed and transferred to a new 1.7 ml tube.
26. 175 μ1 of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).
27. The tubes were placed on a magnet until the beads pelleted.
28. The beads were washed twice with 200 μ1 of Tagmentation Wash Buffer (TWB, Illumina).
29. The beads were then washed once with 200 μ1 of Resuspension Buffer (RSB, Illumina).
30. The beads were resuspended in 20 μ1 of Milli-Q grade water and transferred to 0.2 ml tubes for the final PCR.
31. 20 μ1 of beads+DNA were combined with 25 μ1 of QSU Mastermix (NEB) and 5 μ1 of PPC (PCR Primer Cocktail, Illumina).
32. The mixture was amplified by PCR: cycling procedure—98 C for 3 min followed by 12 cycles of (98 C 45 s, 60 C 2 min, 68 C 2 min), then 68 C for 5 mins and then hold at 4 C.
33. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq.
1. 400 μ1 BspQI mix was made up-380 μl Milli-Q grade water, 40 μ1 of rNEB3.1 buffer (NEB) and 8 μ1 of Nt.BspQI (NEB were combined). The mixture was vortexed to mix and briefly spun down. The mixture was pipetted into the “EXT” position of the MiniSeq cartridge (position to the left of the Custom Primer positions).
2. The library was denatured (0.1N NaOH) and diluted to 0.5 μM final concentration in HT1 buffer according to Illumina protocol. 500 μ1 was loaded into the “Library” position of the MiniSeq cartridge.
3. Setup was run using MiniSeq Control Software, using a standard MiniSeq run.
4. For a CA dye swap, standard IMX was removed from the IMX position of the MiniSeq cartridge, then the position was washed 5 times with Milli-Q grade water, and replaced with 20 mins of custom IMX, where the standard two-dye system for A (A represented by red and green) and one-dye system for C (C represented by red) is replaced with a two-dye system for C (C represented by red and green) and one-dye system for A (A represented by red).

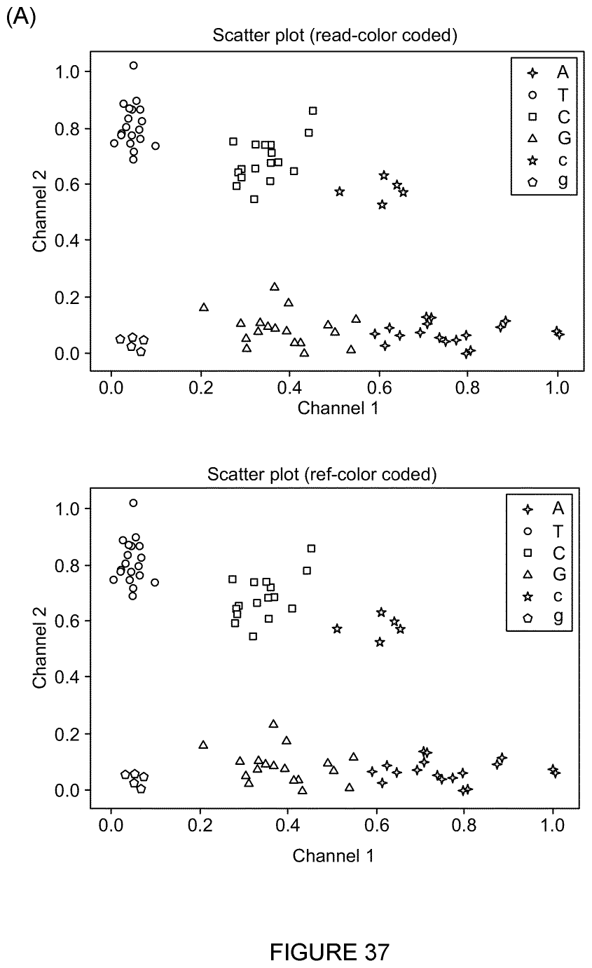

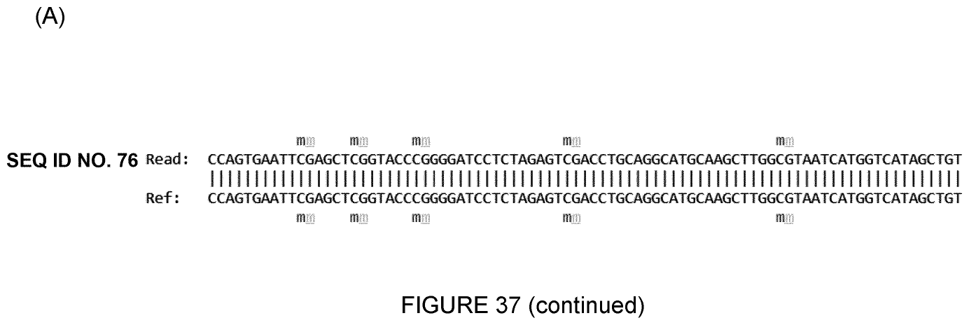

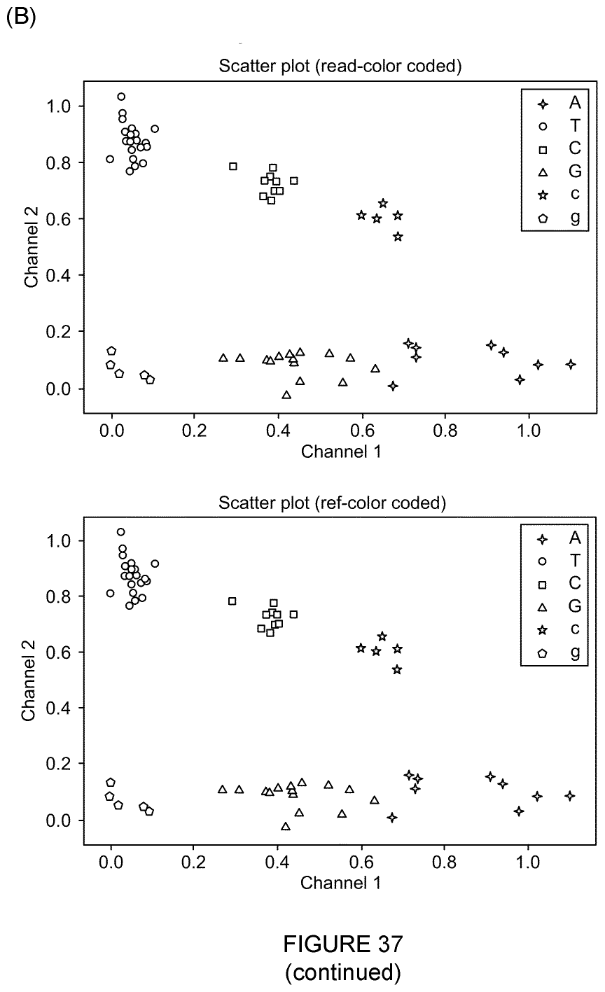

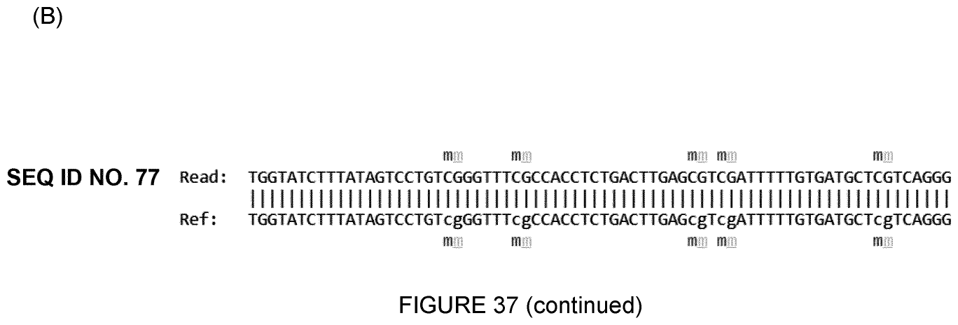

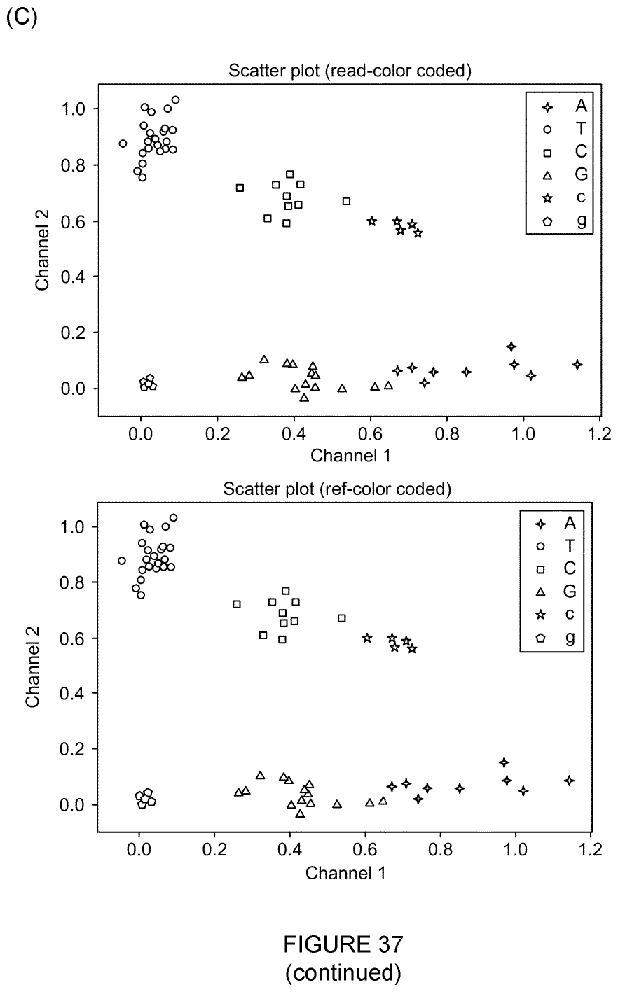

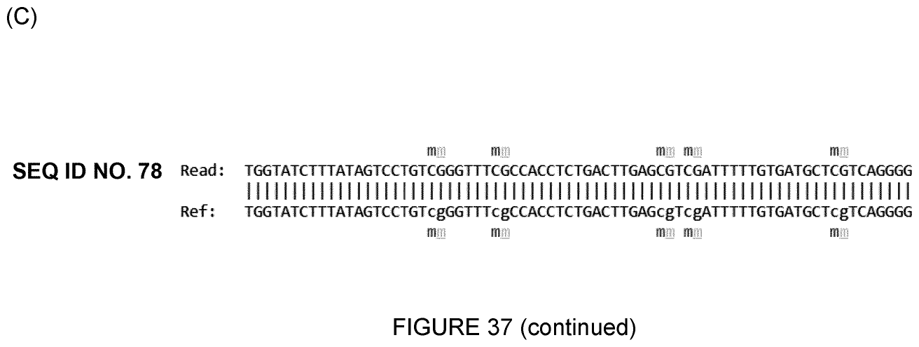

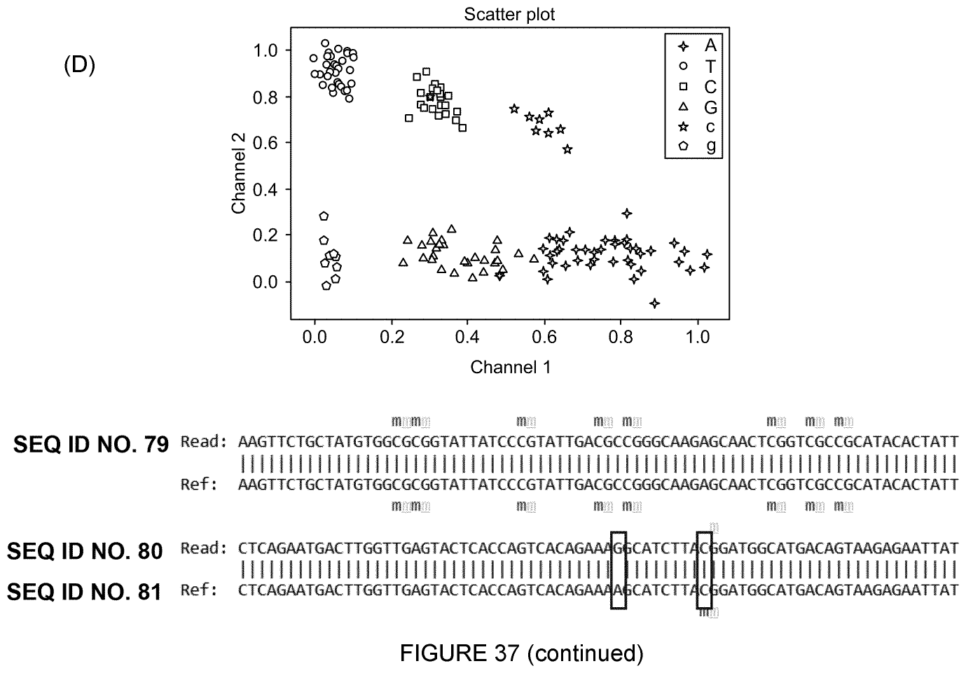

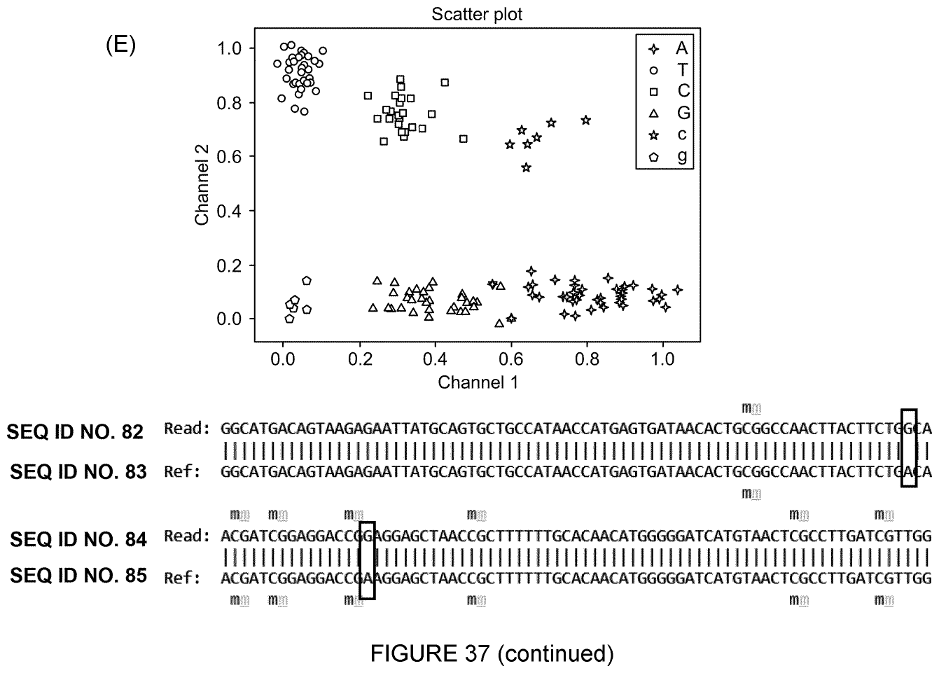

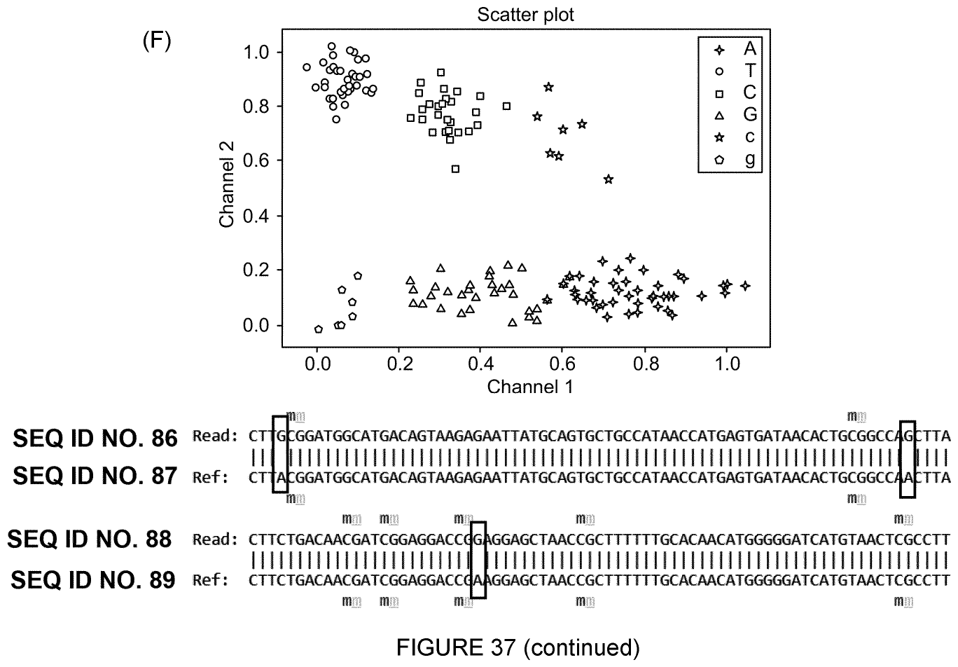

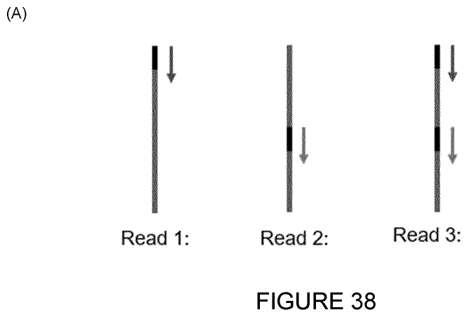

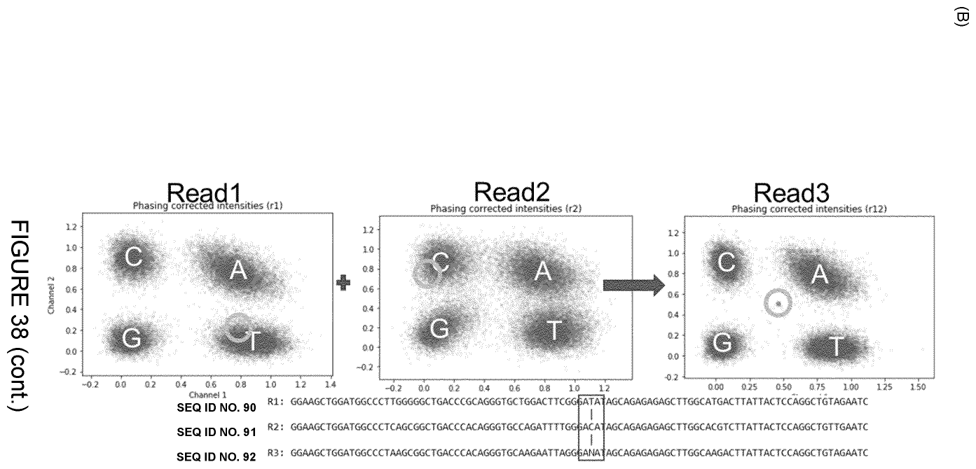

The 9 QaM results are shown in FIGS. 37A to 37F for six different library fragments, where modified cytosines can be identified by characteristic clouds in the top right corner and the bottom left corner in the plot. If the original strands in the library contained a (5mC)-G base pair (the first base corresponding to the forward strand of the library polynucleotide, and the second base corresponding to the reverse strand of the library polynucleotide), this corresponds to a C-G base pair after bisulfite conversion. As such, the forward strand of the template provides a C read (as the forward strand of the template has a G at the corresponding position), and the reverse complement strand of the template provides a C read too (as the reverse complement strand of the template has a G at the corresponding position too), which therefore appears in the top right corner of the plots in FIGS. 37A to 37F (a (C,C) read).

In addition, if the original strands in the library contained a G-(5mC) base pair (the first base corresponding to the forward strand of the library polynucleotide, and the second base corresponding to the reverse strand of the library polynucleotide), this corresponds to a G-C base pair after bisulfite conversion. As such, the forward strand of the template provides a G read (as the forward strand of the template has a C at the corresponding position), and the reverse complement strand of the template provides a G read too (as the reverse complement strand of the template has a C at the corresponding position too), which therefore appears in the bottom left corner of the plots in FIGS. 37A to 37F (a (G,G) read).

By contrast, if the original strands in the library contained a C-G base pair (the first base corresponding to the forward strand of the library polynucleotide, and the second base corresponding to the reverse strand of the library polynucleotide), this corresponds to a T-G mismatched base pair after bisulfite conversion (where C is converted to U, and U is read as T). As such, the forward strand of the template provides a T read (as the forward strand of the template has an A at the corresponding position), and the reverse complement strand of the template provides a C read (as the reverse complement strand of the template has a G at the corresponding position), which therefore appears in the top middle portion of the plots in FIGS. 37A to 37F (a (T,C) read).

If the original strands in the library contained a G-C base pair (the first base corresponding to the forward strand of the library polynucleotide, and the second base corresponding to the reverse strand of the library polynucleotide), this corresponds to a G-T mismatched base pair after bisulfite conversion (where C is converted to U, and U is read as T). As such, the forward strand of the template provides a G read (as the forward strand of the template has a C at the corresponding position), and the reverse complement strand of the template provides an A read (as the reverse complement strand of the template has a T at the corresponding position), which therefore appears in the bottom middle portion of the plots in FIGS. 37A to 37F (a (G,A) read).

If the original strands in the library contained a T-A base pair (the first base corresponding to the forward strand of the library polynucleotide, and the second base corresponding to the reverse strand of the library polynucleotide), this remains as a T-A base pair after bisulfite conversion. As such, the forward strand of the template provides a T read (as the forward strand of the template has an A at the corresponding position), and the reverse complement strand of the template provides a T read too (as the reverse complement strand of the template has an A at the corresponding position too), which therefore appears in the top left corner of the plots in FIGS. 36A to 36F (a (T,T) read).

Finally, if the original strands in the library contained an A-T base pair (the first base corresponding to the forward strand of the library polynucleotide, and the second base corresponding to the reverse strand of the library polynucleotide), this remains as an A-T base pair after bisulfite conversion. As such, the forward strand of the template provides an A read (as the forward strand of the template has a Tat the corresponding position), and the reverse complement strand of the template provides an A read too (as the reverse complement strand of the template has a T at the corresponding position too), which therefore appears in the bottom right corner of the plots in FIGS. 37A to 37F (an (A,A) read).

| Library | Accuracy | | Sensitivity | | Specificity | |
|---|---|---|---|---|---|---|
| Library fragment 1 | 85/85 | (100%) | 10/10 | (100%) | 75/75 | (100%) |
| (FIG. 37A) | | | | | | |
| Library fragment 2 | 72/72 | (100%) | 10/10 | (100%) | 62/62 | (100%) |
| (FIG. 37B) | | | | | | |
| Library fragment 3 | 73/73 | (100%) | 10/10 | (100%) | 63/63 | (100%) |
| (FIG. 37C) | | | | | | |
| Library fragment 4 | 148/150 | (98.67%) | 17/18 | (94.44%) | 133/133 | (100%) |
| (FIG. 37D) | | | | | | |
| Library fragment 5 | 148/150 | (98.67%) | 14/14 | (100%) | 136/136 | (100%) |
| (FIG. 37E) | | | | | | |
| Library fragment 6 | 147/150 | (98%) | 14/14 | (100%) | 136/136 | (100%) |
| (FIG. 37F) | | | | | | |

(Accuracy = number of correct base calls (GCAT, irrespective of methylation status)/total number of bases; Sensitivity= number of true positive methylated base calls/total number of methylated bases; Specificity = number of true negative methylated base calls/(number of true negative methylated base calls + number of false positive methylated base calls))

Overall, these results show that methylation analysis can be conducted on polynucleotide sequences to identify modified cytosines. In particular, by enabling concurrent sequencing of the forward and reverse complement strands of the template (or reverse and forward complement strands of the template), modified cytosines can be identified quickly and accurately.

### Example 3—Mismatched Base Pair Analysis on Human DNA Sample Using 9 QaM

A similar experiment to Example 1 was conducted except that the DNA during the “Tagmentation” section was replaced with a Promega human blend DNA spiked with 5% PhiX (as control). In addition, the steps from “Methylation analysis conversion method” were not conducted—thus, any errors would be indicative of mismatched base pairs, for example, as a result of errors resulting from library preparation.

Sequencing was conducted on the NextSeq 2000. A custom hyb was conducted where the usual primer mix was replaced with HP10 primer mix (Illumina) spiked with HYB2′-ME primer (0.3 μM each). These primers are all unblocked and allow concurrent sequencing of both the first portion and the second portion, and so generate the 9 QaM signal during sequencing. The library was loaded onto the NextSeq 2000 at 650 μM final concentration. These results are presented in FIG. 15B (Read 3—combined Read 1 and Read 2), where mismatched base pairs can be identified by characteristic off-coiner clouds in the plot (indicated by point in circled region). In this case, a C-T mismatch (a middle cloud) was detected, leading to an “N” readout in the Read 3 sequence.

Control experiments were also conducted where individual reads were done separately using only one sequencing primer type (Read 1 and Read 2 separately). One of the reads on the tandem insert corresponds to a readout for the forward strand, whilst the other read on the tandem insert corresponds to a readout for the reverse complement strand. In the Read 1 case, using a HP21 primer mix (Illumina), one of the bases is detected as T (indicated by point in circled region); in the Read 2 case, using a HYB2′-ME primer, one of the bases is detected as C (indicated by point in circled region). The control experiment confirms that the detection of the C-T mismatch in the Read 3 case was correct, using only one read run.

Overall, these results show that analysis can be conducted on polynucleotide sequences to find mismatched base pairs. Again, by enabling concurrent sequencing of the forward and reverse complement strands of the template (or reverse and forward complement strands of the template), mismatched base pairs can be identified quickly and accurately.

1. A method of determining sequence information from two or more polynucleotide sequence portions, the method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the at least one first polynucleotide sequence portion and the at least one second polynucleotide sequence portion.

2. The method of claim 1, wherein the first and second signals and/or the third and fourth signals are obtained substantially simultaneously.

3. The method of claim 1, wherein selecting the classification based on the first and second intensity data comprises selecting the classification based on the combined intensity of the first and second signals and the combined intensity of the third and fourth signals.

4. The method of claim 1, wherein, when based on a nucleobase of the same identity, an intensity of the first signal is substantially the same as an intensity of the second signal and an intensity of the third signal is substantially the same as an intensity of the fourth signal.

5. The method of claim 1, wherein the plurality of classifications consists of a predetermined number of classifications.

6. The method of claim 1, wherein the plurality of classifications comprises:
one or more classifications representing matching first and second nucleobases; and
one or more classifications representing mismatching first and second nucleobases, and wherein determining sequence information from the at least one first polynucleotide sequence portion and the at least one second polynucleotide sequence portion comprises:
in response to selecting a classification representing matching first and second nucleobases, determining a match between the first and second nucleobases; or
in response to selecting a classification representing mismatching first and second nucleobases, determining a mismatch between the first and second nucleobases.

7. The method of claim 6, wherein determining sequence information from the at least one first polynucleotide sequence portion and the at least one second polynucleotide sequence portion comprises, in response to selecting a classification representing a match between the first and second nucleobases, base calling the first and second nucleobases.

8. The method of claim 1, wherein determining sequence information from the at least one first polynucleotide sequence portion and the at least one second polynucleotide sequence portion comprises, based on the selected classification, determining that the second polynucleotide sequence portion is modified relative to the first polynucleotide sequence portion at a location associated with the first and second nucleobases.

9. (canceled)

10. (canceled)

11. The method of claim 1, wherein at least one polynucleotide sequence comprises the first polynucleotide sequence portion and the second polynucleotide sequence portion.

12. (canceled)

13. The method of claim 1, wherein at least one first polynucleotide sequence comprises the first polynucleotide sequence portion and at least one second polynucleotide sequence comprises the second polynucleotide sequence portion.

14. (canceled)

15. The method of claim 1, wherein the first signal, second signal, third signal and fourth signal are generated based on light emissions associated with the respective nucleobase and detected at a sensor, and wherein the obtained signals are generated by:
contacting a plurality of polynucleotide molecules comprising the first and second polynucleotide sequence portions with first primers for sequencing the first polynucleotide sequence portion and second primers for sequencing the second polynucleotide sequence portion;
extending the first primers and the second primers by contacting the polynucleotide molecules with labeled nucleobases to form first labeled primers and second labeled primers;
stimulating the light emissions from the first and second labeled primers; and
detecting the light emissions at a sensor.

16. (canceled)

17. The method of claim 15, wherein:
the first and second signals are based on light emissions detected in a first range of optical frequencies;
the third and fourth signals are based on light emissions detected in a second range of optical frequencies; and
wherein the first range of optical frequencies and the second range of optical frequencies are not identical.

18. The method of claim 15, wherein the polynucleotide molecules comprising the first and second polynucleotide sequence portions are attached to a substrate, optionally a flow cell, and wherein the light emissions from the first labeled primers and the light emissions from the second labeled primers are emitted from the same region or substantially overlapping regions of the substrate.

19. (canceled)

20. The method of claim 15, wherein the light emissions detected at the sensor are spatially unresolved.

21. The method of claim 20, wherein the sensor is configured to provide a single output based upon the first and second signals.

22. The method of claim 15, wherein the sensor comprises a single sensing element.

23. The method of claim 1, wherein the at least one first polynucleotide sequence portion and the at least one second polynucleotide sequence portion are present in a cluster.

24. The method of claim 1, wherein the one of the plurality of classifications is selected based on the first and the second intensity data using a Gaussian mixture model.

25-28. (canceled)

29. A data processing system comprising means for carrying out a method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the at least one first polynucleotide sequence portion and the at least one second polynucleotide sequence portion.

30-31. (canceled)

32. A computer-readable storage medium comprising instructions which, when executed by a processor, cause the processor to carry out a method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the at least one first polynucleotide sequence portion and the at least one second polynucleotide sequence portion.

33-34. (canceled)


SEQUENCE LISTING (92 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 24 bp, synthetic construct
  source 1..24: mol_type = other DNA
caagcagaagacggcatacgagat

SEQ ID NO: 3 - DNA, 29 bp, synthetic construct
  source 1..29: mol_type = other DNA
gtgtagatctcggtggtcgccgtatcatt

SEQ ID NO: 4 - DNA, 24 bp, synthetic construct
  source 1..24: mol_type = other DNA
atctcgtatgccgtcttctgcttg

SEQ ID NO: 5 - DNA, 17 bp, synthetic construct
  source 1..17: mol_type = other DNA
aatgatacggcgaccga

SEQ ID NO: 6 - DNA, 17 bp, synthetic construct
  source 1..17: mol_type = other DNA
tcggtcgccgtatcatt

SEQ ID NO: 7 - DNA, 14 bp, synthetic construct
  source 1..14: mol_type = other DNA
tcgtcggcagcgtc

SEQ ID NO: 8 - DNA, 15 bp, synthetic construct
  source 1..15: mol_type = other DNA
gtctcgtgggctcgg

SEQ ID NO: 9 - DNA, 33 bp, synthetic construct
  source 1..33: mol_type = other DNA
tcgtcggcagcgtcagatgtgtataagagacag

SEQ ID NO: 10 - DNA, 34 bp, synthetic construct
  source 1..34: mol_type = other DNA
gtctcgtgggctcggagatgtgtataagagacag

SEQ ID NO: 11 - DNA, 24 bp, synthetic construct
  source 1..24: mol_type = other DNA
gagtaagtggaagagataggaagg

SEQ ID NO: 12 - DNA, 43 bp, synthetic construct
  source 1..43: mol_type = other DNA
gagtaagtggaagagataggaaggagatgtgtataagagacag

SEQ ID NO: 13 - DNA, 24 bp, synthetic construct
  source 1..24: mol_type = other DNA
ccttcctatctcttccacttactc

SEQ ID NO: 14 - DNA, 43 bp, synthetic construct
  source 1..43: mol_type = other DNA
ccttcctatctcttccacttactcagatgtgtataagagacag

SEQ ID NO: 15 - DNA, 23 bp, synthetic construct
  source 1..23: mol_type = other DNA
  misc_binding 23: bound_moiety = 3' propanol
ccttcctatctcttccacttact

SEQ ID NO: 16 - DNA, 43 bp, synthetic construct; identical to SEQ ID NO: 14
  source 1..43: mol_type = other DNA
  misc_binding 43: bound_moiety = 3' propanol

SEQ ID NO: 17 - DNA, 33 bp, synthetic construct
  source 1..33: mol_type = other DNA
ctgtctcttatacacatctgacgctgccgacga

SEQ ID NO: 18 - DNA, 14 bp, synthetic construct
  source 1..14: mol_type = other DNA
gacgctgccgacga

SEQ ID NO: 19 - DNA, 34 bp, synthetic construct
  source 1..34: mol_type = other DNA
ctgtctcttatacacatctccgagcccacgagac

SEQ ID NO: 20 - DNA, 15 bp, synthetic construct
  source 1..15: mol_type = other DNA
ccgagcccacgagac

SEQ ID NO: 21 - DNA, 43 bp, synthetic construct
  source 1..43: mol_type = other DNA
ctgtctcttatacacatctgagtaagtggaagagataggaagg

SEQ ID NO: 22 - DNA, 43 bp, synthetic construct
  source 1..43: mol_type = other DNA
ctgtctcttatacacatctccttcctatctcttccacttactc

SEQ ID NO: 23 - protein, 198 aa, Homo sapiens [sequence omitted]

SEQ ID NO: 24 - protein, 188 aa, Gorilla gorilla
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 25 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 26 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 27 - protein, 191 aa, Gorilla gorilla
MSRKIWRSSGKNTTNHVEVNFIKKFTSERHFHPSISCSITWFLSWSPCWECSQAIREFLSQHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 28 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 29 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 30 - protein, 230 aa, Gorilla gorilla
MAQKEEAAAATEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 31 - protein, 224 aa, Alphapolyomavirus muris
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 32 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 33 - protein, 202 aa, Macaca fascicularis
MDGSPASRPRHLMDPNTFTFNFNNDLSVRGRHQTYLCYEVERLDNGTWVPMDERRGFLHNKAKNVPCGDYGCHVELRFLCEVPSWQLDPAQTYRVTWFISWSPCFRKGCAGQVRAFLQENKHVRLRIFAARIYDYDPRYQEALRTLRDAGAQVSIMTYEEFKHCWDTFVDRQGRPFQPWDGLDEHSQALSGRLRAILQNQGN

SEQ ID NO: 34 - protein, 199 aa, Pongo pygmaeus
MEASPASGPRHLMDPCVFTSNFNNGIRWHKTYLCYEVERLDNGTWVKMDQHRGFLHNQARNPLYGLDGRHAELRFLGLLPYWQLDPAQIYRVTWFISWSPCFSWGCARQVRAFLQENTHVRLRIFAARIYDYDPLYKEALQMLRDAGAQVSIMTYDEFEYCWNTFVDHQGCPFQPWDGLEEHSQALSGKLQAILLNQGN

SEQ ID NO: 35 - protein, 199 aa, Nomascus leucogenys
MEASPASRPGHLMDPQVFTSNFNNGIRWHKTYLCYEVERLDNGTWVKMDQHRGFLHNQAKNLFCGFYGRHAELCFLDLVPSLQLDPAQTYRVTWFISWSPCFSWGCAEQVRAFLQENTHVRLRLFAARIYDYDPLYKEALQMLRGAGAQVSIMTYHEFKHCWDTFVDHQGRPFQPWDGLEEHSQALSGRLQAILQNQGN

SEQ ID NO: 36 - protein, 199 aa, Pan troglodytes
TEASPASGPRHLMDPHIFTSNFNNGIGRRKTYLCYEVERLDNGTSVKMDQHRGFLHNQAKNLLCGFYGRHAELCFLDLVPSLQLDPAQIYRVTWFISWSPCFSWGCAGQVRAFLQENTHVRLRIFAARIYDYDPLYKEALQMLRDAGAQVSIMTYDEFKHCWDTFVDHQGCPFQPWDGLEEHSQALSGRLRAILQNQGN

SEQ ID NO: 37 - protein, 199 aa, Gorilla gorilla
VEASPASGPRHLMDPHIFTSNFNNVIGRHKTYLCYEVERLDNGTWVKMDQHRGFLHNQAKNLLCGFYGRHAELRFLDLVPSLQLDPAQIYRVTWFISWSPCFSWGCAGQVRAFLQENTHVRLHIFAARIYDYDPLYKEALQMLRDAGAQVSIMTYDEFKHCWDTFVDHQGCPFQPWDGLEEHSQALSGRLRAILQNQGN

SEQ ID NO: 38 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 39 - protein, 382 aa, Gorilla gorilla
MNPQIRNPMERMYRGTFYNNFENEPILYGRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRLSQAGARMKIMDYEECAYCWENFVYKEGQQFMPWYKFDENYAFLHHTLKEILRHLMDPDTFTFNFNNDPLVLRRHQTYLCYEVERLDNGTWVLMDRHMGFLCNEAKNLLCGFYGRHAELRFLDLVPSLQLDPAQIYRVTWFISWSPCFSWGCAGQVCEFLQENTHMRLRIFAARIYDYDPLYKKALQMLRDAGAQVSIMTYDEFKHCWDTFVYRQGCPFQPWDGLEEHSQALSGRLQAILQNQGN

SEQ ID NO: 40 - protein, 382 aa, Macaca mulatta
MNPQIRNPMERMYRRTFNYNFENEPILYGRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRLRQAGARVKIMDYEEFAYCWENFVYNEDQSFMPWYKFDDNYAFLHHKLKEILRHLMDPDTFTSNFNNDLSVLGRHQTYLCYEVERLDNGTWVPMDQHWGFLCNQAKNVPRGDYGCHAELCFLDQVSSWQLDPAQTYRVTWFISWSPCFSWGCADQVYAFLQENTHVRLRIFAARIYDYNPLYQEALRTLRDAGAQVSIMTYDEFEYCWDTFVDRQGRPFQPWDGLDEHSQALSGRLRAILQNQGN

SEQ ID NO: 41 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 42 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 43 - protein, 190 aa, Macaca mulatta
MNPQIRNPMKAMDPGTFYFQFKNLWEANNRNETWLCFTVEVIKQHSTVSWETGVFRNQVDLETHCHAERCFLSWFCEDILSPNTDYQVTWYTSWSPCLDCAGEVAKFLARHNNVMLTIYTARLYYSQYPNYQQGLRSLSEKGVSVKIMDYEDFKYCWEKFVYDDGEPFKPWKGLKTSFRFLKRRLREILQ

SEQ ID NO: 44 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 45 - protein, 386 aa, Pan troglodytes
MNPQIRNPMERMYRRTFYNHFENEPILYGRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRLHKAGARVKIMDYKDFAHCWENFVYNEGQQFMPWYKFDDNYASLHRTLKEILRNPMEAMYPHVFYFHFKNLLKACGRNESWLCFTVDVTEHHPPVSWKRGVFRNPVDPETHCHAERCFLSWFCDDILSPNTNYQVTWYTSWSPCPECAREVAEFLARHSNVKLTIFTARLYHFWNTDYQEGLCSLSQEGASVKIMSYKDFVSCWKNFVYSDDEPFKPWKGLKTNFRLLKTMLREILQ

SEQ ID NO: 46 - protein, 374 aa, Macaca mulatta
MNPQIRNPMERMYRRTFNYNFENEPILYGRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRLHQAGARVKIMDYEEFAYCWENFVYNEGQSFMPWDKFDDNYAFLHHKLKEILRNPMKAMYPHTFYFHFENLQKAYGRNETWLCFAVEIIKQHSTVPWKTGVFRNQVDPETHCHAERCFLSWFCDNTLSPKKNYQVTWYISWSPCPECAGEVAEFLATHSNVKLTIYTARLYYFWDTDYQEGLRSLSEEGASMEIMGYEDFKYCWENFVYNDGEPFKPWKGINTNFRFLERRLWKILQ

SEQ ID NO: 47 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 48 - protein, 373 aa, Gorilla gorilla
MKPQFRNTVERMYRGTFSYNFNNRPILSRRNTVWLCYEVKTKGPSRPPLDAKIFRGQVYFQPQYHAEMCFLSWFCGNQLPAYKCFQITCFVSWTPCPDCVAKLAEFLAEHPNVTLTISAARLYYYWERDYRRALRRLRQAGARVKIMDDEEFAYCWENFVYSEGQPFMPWHKFDDNYAFLHRTLKEILRNPMEAMYPHIFYFHFKNLLKAYGRNESWLCFTMEVIKHHSPVSWKRGVFRNQVDSETHCHAERCFLSWFCDDILSPNTNYQVTWYTSWSPCPECAGEVAEFLARHSNVNLTIFTARLYYFWDTDYQEGLRSLNQEGASVKIMGYKDFKYCWENFVYNDDEPFKPWKGLKYNFLFLDSKLQEILE

SEQ ID NO: 49 - protein, 373 aa, Macaca mulatta
MQPQYRNTVERMYRGTFFYNFNNRPILSRRNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRLRQAGARVKIMDYEEFAYCWENFVYNEGQSFMPWDKFDDNYAFLHHKLKEILRNPMEATYPHIFYFHFKNLRKAYGRNETWLCFTMEIIKQHSTVSWETGVFRNQVDPESRCHAERCFLSWFCEDILSPNTDYQVTWYTSWSPCLDCAGEVAEFLARHSNVKLAIFAARLYYFWDTHYQQGLRSLSEKGASVEIMGYKDFKYCWENFVYNGDEPFKPWKGLKYNFLFLDSKLQEILE

SEQ ID NO: 50 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 51 - protein, 384 aa, Gorilla gorilla
MTPQFRNTVERMYRDTFSYNFNNRPILSRRNTVWLCYEVKTKDPSRPPLDAKIFRGQVYSELKYHPEMRFFHWFSKWRKLHRDQEYEVTWYISWSPCTKCTRNVATFLAEDPKVTLTIFVARLYYFWDQDYQEALRSLCQKRDGPRATMKIMNYDEFQHCWSKFVYSQRELFEPWNNLPKYYMLLHIMLGEILRHSMDPPTFTSNFNNEHWVRGRHETYLCYEVERLHNDTWVLLNQRRGFLCNQAPHKHGFLEGRHAELCFLDVIPFWKLDLHQDYRVTCFTSWSPCFSCAQEMAKFISNKKHVSLCIFAARIYDDQGRCQEGLRTLAEAGAKISIMTYSEFKHCWDTFVYHQGCPFQPWDGLEEHSQALSGRLQAILQNQGN

SEQ ID NO: 52 - protein, 384 aa, Macaca mulatta
MNPQIRNMVEPMDPRTFVSNFNNRPILSGLNTVWLCCEVKTKDPSGPPLDAKIFQGKVLRSKAKYHPEMRFLQWFREWRQLHHDQEYKVTWYVSWSPCTRCANSVATFLAKDPKVTLTIFVARLYYFWKPNYQQALRILCQKRDGPHATMKIMNYNEFQDCWNKFVDGRGKPFKPWNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRTLHRDGAKIAMMNYSEFEYCWDTFVDCQGCPFQPWDGLDEHSQALSERLRAILQNQGN

SEQ ID NO: 53 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 54 - protein, 210 aa, Gorilla gorilla
MALLTAETFRLQFNNKLRLRRPYYRRKTLLCYQLTPQNGSMPTRGYFKNKKKCHAEICFINEIKSMGLDETQCYQVTCYLTWSPCSSCAWKLVDFIKAHDHLNLRIFASRLYYHWCKRQQEGLRLLCGSQVPVEVMGFPEFADCWENFVDHEKPLSFDPSKMLEELDKNSQAIKRRLERIKSRSVDVLENGLRSLQLGPVTPSSSRSNSR

SEQ ID NO: 55 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 56 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 57 - protein, 368 aa, Gorilla gorilla
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQSFQMPSGQLQPNLPPDVRAPVVFVLVPLRDLPPMHMGQNPNKPRNIVRHLNMPQMSFQETEDLGRLPTGRSVEIVEITERFASSKEADEKKKKKKGKK

SEQ ID NO: 58 - protein, 374 aa, Mus musculus
MEPLYEEILTQGGTIVKPYYWLSLSLGCTNCPYHIRTGEEARVPYTEFHQTFGFPWSTYPQTKHLTFYELRSSSKNLIQKGLASNCTGSHNHPEAMLFEKNGYLDAVIFHNSNIRHIILYSNNSPCNEAKHCCISKMYNFLMNYPEVTLSVFFSQLYHTEKQFPTSAWNRKALQSLASLWPQVTLSPICGGLWHAILEKFVSNISGSTVPQPFIAGRILADRYNTYEINSIIAAKPYFTDGLLSRQKENQNREAWAAFEKHPLGSAAPAQRQPTRGQDPRTPAVLMLVSNRDLPPIHVGSTPQKPRTVVRHLNMLQLSSFKVKDVKKPPSGRPVEEVEVMKESARSQKANKKNRSQWKKQTLVIKPRICRLLER

SEQ ID NO: 59 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 60 - protein, 2007 aa, Mus musculus
MSRSRPAKPSKSVKTKLQKKKDIQMKTKTSKQAVRHGASAKAVNPGKPKQLIKRRDGKKETEDKTPTPAPSFLTRAGAARMNRDRNQVLFQNPDSLTCNGFTMALRRTSLSWRLSQRPVVTPKPKKVPPSKKQCTHNIQDEPGVKHSENDSVPSQHATVSPGTENGEQNRCLVEGESQEITQSCPVFEERIEDTQSCISASGNLEAEISWPLEGTHCEELLSHQTSDNECTSPQECAPLPQRSTSEVTSQKNTSNQLADLSSQVESIKLSDPSPNPTGSDHNGFPDSSFRIVPELDLKTCMPLDESVYPTALIRFILAGSQPDVFDTKPQEKTLITTPEQVGSHPNQVLDATSVLGQAFSTLPLQWGFSGANLVQVEALGKGSDSPEDLGAITMLNQQETVAMDMDRNATPDLPIFLPKPPNTVATYSSPLLGPEPHSSTSCGLEVQGATPILTLDSGHTPQLPPNPESSSVPLVIAANGTRAEKQFGTSLFPAVPQGFTVAAENEVQHAPLDLTQGSQAAPSKLEGEISRVSITGSADVKATAMSMPVTQASTSSPPCNSTPPMVERRKRKACGVCEPCQQKANCGECTYCKNRKNSHQICKKRKCEVLKKKPEATSQAQVTKENKRPQREKKPKVLKTDFNNKPVNGPKSESMDCSRRGHGEEEQRLDLITHPLENVRKNAGGMTGIEVEKWAPNKKSHLAEGQVKGSCDANLTGVENPQPSEDDKQQTNPSPTFAQTIRNGMKNVHCLPTDTHLPLNKLNHEEFSKALGNNSSKLLTDPSNCKDAMSVTTSGGECDHLKGPRNTLLFQKPGLNCRSGAEPTIFNNHPNTHSAGSRPHPPEKVPNKEPKDGSPVQPSLLSLMKDRRLTLEQVVAIEALTQLSEAPSESSSPSKPEKDEEAHQKTASLLNSCKAILHSVRKDLQDPNVQGKGLHHDTVVFNGQNRTFKSPDSFATNQALIKSQGYPSSPTAEKKGAAGGRAPFDGFENSHPLPIESHNLENCSQVLSCDQNLSSHDPSCQDAPYSQIEEDVAAQLTQLASTINHINAEVRNAESTPESLVAKNTKQKHSQEKRMVHQKPPSSTQTKPSVPSAKPKKAQKKARATPHANKRKKKPPARSSQENDQKKQEQLAIEYSKMHDIWMSSKFQRFGQSSPRSFPVLLRNIPVFNQILKPVTQSKTPSQHNELFPPINQIKFTRNPELAKEKVKVEPSDSLPTCQFKTESGGQTFAEPADNSQGQPMVSVNQEAHPLPQSPPSNQCANIMAGAAQTQFHLGAQENLVHQIPPPTLPGTSPDTLLPDPASILRKGKVLHFDGITVVTEKREAQTSSNGPLGPTTDSAQSEFKESIMDLLSKPAKNLIAGLKEQEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWV

SEQ ID NO: 61 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 62 - protein, 1912 aa, Mus musculus
MEQDRTTHAEGTRLSPFLIAPPSPISHTEPLAVKLQNGSPLAERPHPEVNGDTKWQSSQSCYGISHMKGSQSSHESPHEDRGYSRCLQNGGIKRTVSEPSLSGLHPNKILKLDQKAKGESNIFEESQERNHGKSSRQPNVSGLSDNGEPVTSTTQESSGADAFPTRNYNGVEIQVLNEQEGEKGRSVTLLKNKIVLMPNGATVSAHSEENTRGELLEKTQCYPDCVSIAVQSTASHVNTPSSQAAIELSHEIPQPSLTSAQINFSQTSSLQLPPEPAAMVTKACDADNASKPAIVPGTCPFQKAEHQQKSALDIGPSRAENKTIQGSMELFAEEYYPSSDRNLQASHGSSEQYSKQKETNGAYFRQSSKFPKDSISPTTVTPPSQSLLAPRLVLQPPLEGKGALNDVALEEHHDYPNRSNRTLLREGKIDHQPKTSSSQSLNPSVHTPNPPLMLPEQHQNDCGSPSPEKSRKMSEYLMYYLPNHGHSGGLQEHSQYLMGHREQEIPKDANGKQTQGSVQAAPGWIELKAPNLHEALHQTKRKDISLHSVLHSQTGPVNQMSSKQSTGNVNMPGGFQRLPYLQKTAQPEQKAQMYQVQVNQGPSPGMGDQHLQFQKALYQECIPRTDPSSEAHPQAPSVPQYHFQQRVNPSSDKHLSQQATETQRLSGFLQHTPQTQASQTPASQNSNFPQICQQQQQQQLQRKNKEQMPQTFSHLQGSNDKQREGSCFGQIKVEESFCVGNQYSKSSNFQTHNNTQGGLEQVQNINKNFPYSKILTPNSSNLQILPSNDTHPACEREQALHPVGSKTSNLQNMQYFPNNVTPNQDVHRCFQEQAQKPQQASSLQGLKDRSQGESPAPPAEAAQQRYLVHNEAKALPVPEQGGSQTQTPPQKDTQKHAALRWLLLQKQEQQQTQQSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 63 - protein, 1795 aa, Homo sapiens
MSQFQVPLAVQPDLPGLYDFPQRQVMVGSFPGSGLSMAGSESQLRGGGDGRKKRKRCGTCEPCRRLENCGACTSCTNRRTHQICKLRKCEVLKKKVGLLKEVEIKAGEGAGPWGQGAAVKTGSELSPVDGPVPGQMDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 64 - protein, 1803 aa, Mus musculus
MSQFQVPLAVQPDLSGLYDFPQGQVMVGGFQGPGLPMAGSETQLRGGGDGRKKRKRCGTCDPCRRLENCGSCTSCTNRRTHQICKLRKCEVLKKKAGLLKEVEINAREGTGPWAQGATVKTGSELSPVDGPVPGQMDSGPVYHGDSRQLSTSGAPVNGAREPAGPGLLGAAGPWRVDQKPDWEAASGPTHAARLEDAHDLVAFSAVAEAVSSYGALSTRLYETFNREMSREAGSNGRGPRPESCSEGSEDLDTLQTALALARHGMKPPNCTCDGPECPDFLEWLEGKIKSMAMEGGQGRPRLPGALPPSEAGLPAPSTRPPLLSSEVPQVPPLEGLPLSQSALSIAKEKNISLQTAIAIEALTQLSSALPQPSHSTSQASCPLPEALSPSAPFRSPQSYLRAPSWPVVPPEEHPSFAPDSPAFPPATPRPEFSEAWGTDTPPATPRNSWPVPRPSPDPMAELEQLLGSASDYIQSVFKRPEALPTKPKVKVEAPSSSPAPVPSPISQREAPLLSSEPDTHQKAQTALQQHLHHKRNLFLEQAQDASFPTSTEPQAPGWWAPPGSPAPRPPDKPPKEKKKKPPTPAGGPVGAEKTTPGIKTSVRKPIQIKKSRSRDMQPLFLPVRQIVLEGLKPQASEGQAPLPAQLSVPPPASQGAASQSCATPLTPEPSLALFAPSPSGDSLLPPTQEMRSPSPMVALQSGSTGGPLPPADDKLEELIRQFEAEFGDSFGLPGPPSVPIQEPENQSTCLPAPESPFATRSPKKIKIESSGAVTVLSTTCFHSEEGGQEATPTKAENPLTPTLSGFLESPLKYLDTPTKSLLDTPAKKAQSEFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTACMDFCAHAHKDQHNLYNGCTVVCTLTKEDNRCVGQIPEDEQLHVLPLYKMASTDEFGSEENQNAKVSSGAIQVLTAFPREVRRLPEPAKSCRQRQLEARKAAAEKKKLQKEKLSTPEKIKQEALELAGVTTDPGLSLKGGLSQQSLKPSLKVEPQNHFSSFKYSGNAVVESYSVLGSCRPSDPYSMSSVYSYHSRYAQPGLASVNGFHSKYTLPSFGYYGFPSSNPVFPSQFLGPSAWGHGGSGGSFEKKPDLHALHNSLNPAYGGAEFAELPGQAVATDNHHPIPHHQQPAYPGPKEYLLPKVPQLHPASRDPSPFAQSSSCYNRSIKQEPIDPLTQAESIPRDSAKMSRTPLPEASQNGGPSHLWGQYSGGPSMSPKRTNSVGGNWGVFPPGESPTIVPDKLNSFGASCLTPSHFPESQWGLFTGEGQQSAPHAGARLRGKPWSPCKFGNGTSALTGPSLTEKPWGMGTGDFNPALKGGPGFQDKLWNPVKVEEGRIPTPGANPLDKAWQAFGMPLSSNEKLFGALKSEEKLWDPFSLEEGTAEEPPSKGVVKEEKSGPTVEEDEEELWSDSEHNFLDENIGGVAVAPAHCSILIECARRELHATTPLKKPNRCHPTRISLVFYQHKNLNQPNHGLALWEAKMKQLAERARQRQEEAARLGLGQQEAKLYGKKRKWGGAMVAEPQHKEKKGAIPTRQALAMPTDSAVTVSSYAYTKVTGPYSRWI

SEQ ID NO: 65 - protein, 400 aa, Tequatrovirus T4
MRICIFMARGLEGCGVTKFSLEQRDWFIKNGHEVTLVYAKDKSFTRTSSHDHKSFSIPVILAKEYDKALKLVNDCDILIINSVPATSVQEATINNYKKLLDNIKPSIRVVVYQHDHSVLSLRRNLGLEETVRRADVIFSHSDNGDFNKVLMKEWYPETVSLFDDIEEAPTVYNFQPPMDIVKVRSTYWKDVSEINMNINRWIGRTTTWKGFYQMFDFHEKFLKPAGKSTVMEGLERSPAFIAIKEKGIPYEYYGNREIDKMNLAPNQPAQILDCYINSEMLERMSKSGFGYQLSKLNQKYLQRSLEYTHLELGACGTIPVFWKSTGENLKFRVDNTPLTSHDSGIIWFDENDMESTFERIKELSSDRALYDREREKAYEFLYQHQDSSFCFKEQFDIITK

SEQ ID NO: 66 - protein, 351 aa, Tequatrovirus T4
MKIAIINMGNNVINFKTVPSSETIYLFKVISEMGLNVDIISLKNGVYTKSFDEVDVNDYDRLIVVNSSINFFGGKPNLAILSAQKFMAKYKSKIYYLFTDIRLPFSQSWPNVKNRPWAYLYTEEELLIKSPIKVISQGINLDIAKAAHKKVDNVIEFEYFPIEQYKIHMNDFQLSKPTKKTLDVIYGGSFRSGQRESKMVEFLFDTGLNIEFFGNAREKQFKNPKYPWTKAPVFTGKIPMNMVSEKNSQAIAALIIGDKNYNDNFITLRVWETMASDAVMLIDEEFDTKHRIINDARFYVNNRAELIDRVNELKHSDVLRKEMLSIQHDILNKTRAKKAEWQDAFKKAIDL

SEQ ID NO: 67 - protein, 38 aa, Homo sapiens
  VARIANT 27..31: note = amino acids at positions 26 - 31 are optional
  VARIANT 36..37: note = amino acids at positions 36 - 37 are optional
  VARIANT 2: note = X at position 2 is selected from P, A, and V
  VARIANT 4..31: note = X can be any amino acid
  VARIANT 34..37: note = X can be any amino acid
HXEXXXXXXXXXXXXXXXXXXXXXXXXXXXXPCXXXXC

SEQ ID NO: 68 - protein, 87 aa, Homo sapiens
  VARIANT 2: note = X is any amino acid
  VARIANT 30: note = X at position 30 is selected from S and T
  VARIANT 35..36: note = amino acids at positions 35 and 36 are optional
  VARIANT 70..72: note = amino acids at positions 70 to 72 are optional
  VARIANT 4..27: note = X can be any amino acid
  VARIANT 33..36: note = X can be any amino acid
  VARIANT 38..43: note = X can be any amino acid
  VARIANT 45..52: note = X can be any amino acid
  VARIANT 54..58: note = X can be any amino acid
  VARIANT 62..72: note = X can be any amino acid
  VARIANT 74..75: note = X can be any amino acid
  VARIANT 77..86: note = X can be any amino acid
HXEXXXXXXXXXXXXXXXXXXXXXXXXSWXPCXXXXCXXXXXXFXXXXXXXXLXXXXXRJYXXXXXXXXXXXLXXLXXXXXXXXXXM

SEQ ID NO: 69 - DNA, 70 bp, synthetic construct
  source 1..70: mol_type = other DNA
  modified_base 69^70: mod_base = OTHER; note = phosphorothioate bond
  modified_base 32: mod_base = OTHER; note = uracil
gctgaggatctcgtatgccgtcttctgcttgtaatgatacggcgaccaccgagatctacactcctcagct

SEQ ID NO: 70 - DNA, 69 bp, synthetic construct
  source 1..69: mol_type = other DNA
  modified_base 35: mod_base = OTHER; note = biotin-T
  modified_base 68^69: mod_base = OTHER; note = phosphorothioate bond
gaagagcacacgtctgaactccagtcactagggataacagggtaatctttccctacacgacgctcttct

SEQ ID NO: 71 - DNA, 70 bp, synthetic construct; identical to SEQ ID NO: 69
  source 1..70: mol_type = other DNA
  modified_base 69^70: mod_base = OTHER; note = phosphorothioate bond
  modified_base 2: mod_base = OTHER; note = 5-methylcytosine at position 2
  modified_base 10: mod_base = OTHER; note = 5-methylcytosine at position 10
  modified_base 12: mod_base = OTHER; note = 5-methylcytosine at position 12
  modified_base 18: mod_base = OTHER; note = 5-methylcytosine at position 18
  modified_base 19: mod_base = OTHER; note = 5-methylcytosine at position 19
  modified_base 22: mod_base = OTHER; note = 5-methylcytosine at position 22
  modified_base 25: mod_base = OTHER; note = 5-methylcytosine at position 25
  modified_base 28: mod_base = OTHER; note = 5-methylcytosine at postion 28
  modified_base 40: mod_base = OTHER; note = 5-methylcytosine at position 40
  modified_base 43: mod_base = OTHER; note = 5-methylcytosine at position 43
  modified_base 46: mod_base = OTHER; note = 5-methylcytosine at position 46
  modified_base 47: mod_base = OTHER; note = 5-methylcytosine at position 47
  modified_base 49: mod_base = OTHER; note = 5-methylcytosine at position 49
  modified_base 50: mod_base = OTHER; note = 5-methylcytosine at position 50
  modified_base 56: mod_base = OTHER; note = 5-methylcytosine at position 56
  modified_base 59: mod_base = OTHER; note = 5-methylcytosine at position 59
  modified_base 61: mod_base = OTHER; note = 5-methylcytosine at position 61
  modified_base 63: mod_base = OTHER; note = 5-methylcytosine at position 63
  modified_base 64: mod_base = OTHER; note = 5-methylcytosine at position 64
  modified_base 66: mod_base = OTHER; note = 5-methylcytosine at position 66
  modified_base 69: mod_base = OTHER; note = 5-methylcytosine at position 69
  modified_base 32: mod_base = OTHER; note = uracil at position 32

SEQ ID NO: 72 - DNA, 69 bp, synthetic construct; identical to SEQ ID NO: 70
  source 1..69: mol_type = other DNA
  modified_base 68^69: mod_base = OTHER; note = phosphorothioate bond
  modified_base 9: mod_base = OTHER; note = 5-methylcytosine
  modified_base 11: mod_base = OTHER; mod_base = OTHER; note = 5-methylcytosine
  modified_base 14: mod_base = OTHER; note = 5-methylcytosine
  modified_base 19: mod_base = OTHER; note = 5-methylcytosine
  modified_base 21: mod_base = OTHER; mod_base = OTHER; note = 5-methylcytosine
  modified_base 22: mod_base = OTHER; mod_base = OTHER; note = 5-methylcytosine
  modified_base 26: mod_base = OTHER; note = 5-methylcytosine
  modified_base 28: mod_base = OTHER; mod_base = OTHER; note = 5-methylcytosine
  modified_base 35: mod_base = OTHER; note = Biotin-T
  modified_base 38: mod_base = OTHER; mod_base = OTHER; note = 5-methylcytosine
  modified_base 47: mod_base = OTHER; note = 5-methylcytosine
  modified_base 51: mod_base = OTHER; note = 5-methylcytosine
  modified_base 52: mod_base = OTHER; note = 5-methylcytosine
  modified_base 53: mod_base = OTHER; mod_base = OTHER; note = 5-methylcytosine
  modified_base 56: mod_base = OTHER; mod_base = OTHER; note = 5-methylcytosine
  modified_base 58: mod_base = OTHER; note = 5-methylcytosine
  modified_base 61: mod_base = OTHER; mod_base = OTHER; note = 5-methylcytosine
  modified_base 63: mod_base = OTHER; note = 5-methylcytosine
  modified_base 65: mod_base = OTHER; note = 5-methylcytosine
  modified_base 68: mod_base = OTHER; note = 5-methylcytosine
  modified_base 7: mod_base = OTHER; note = 5-methylcytosine

SEQ ID NO: 73 - DNA, 36 bp, Escherichia coli
  source 1..36: mol_type = genomic DNA; note = pUC19
ccgcctctccccgcgcgttggccgattcattaatgc

SEQ ID NO: 74 - DNA, 36 bp, Escherichia coli
  source 1..36: mol_type = genomic DNA; note = pUC19
ctgcctctccctgtgtgttggctgattcattaatgc

SEQ ID NO: 75 - DNA, 540 bp, Escherichia coli
  source 1..540: mol_type = genomic DNA; note = pUC19
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagt

SEQ ID NO: 76 - DNA, 85 bp, Escherichia coli
  source 1..85: mol_type = genomic DNA; note = pUC19
  modified_base 11: mod_base = OTHER; note = 5-Methylcytosine
  modified_base 17: mod_base = OTHER; note = 5-Methylcytosine
  modified_base 24: mod_base = OTHER; note = 5-Methylcytosine
  modified_base 41: mod_base = OTHER; note = 5-Methylcytosine
  modified_base 65: mod_base = OTHER; note = 5-Methylcytosine
ccagtgaattcgagctcggtacccggggatcctctagagtcgacctgcaggcatgcaagcttggcgtaatcatggtcatagctgt

SEQ ID NO: 77 - DNA, 72 bp, Escherichia coli
  source 1..72: mol_type = genomic DNA; note = pUC19
  modified_base 21: mod_base = OTHER; note = 5-Methylcytosine
  modified_base 28: mod_base = OTHER; note = 5-Methylcytosine
  modified_base 46: mod_base = OTHER; note = 5-Methylcytosine
  modified_base 49: mod_base = OTHER; note = 5-Methylcytosine
  modified_base 65: mod_base = OTHER; note = 5-Methylcytosine
tggtatctttatagtcctgtcgggtttcgccacctctgacttgagcgtcgatttttgtgatgctcgtcaggg

SEQ ID NO: 78 - DNA, 73 bp, Escherichia coli
  source 1..73: mol_type = genomic DNA; note = pUC19
  modified_base 21: mod_base = OTHER; note = 5-Methylcytosine
  modified_base 28: mod_base = OTHER; note = 5-Methylcytosine
  modified_base 46: mod_base = OTHER; note = 5-Methylcytosine
  modified_base 49: mod_base = OTHER; note = 5-Methylcytosine
  modified_base 65: mod_base = OTHER; note = 5-Methylcytosine
tggtatctttatagtcctgtcgggtttcgccacctctgacttgagcgtcgatttttgtgatgctcgtcagggg

SEQ ID NO: 79 - DNA, 75 bp, Escherichia coli
  source 1..75: mol_type = genomic DNA; note = pUC19
  modified_base 17: mod_base = OTHER; note = 5-Methylcytosine
  modified_base 19: mod_base = OTHER; note = 5-Methylcytosine
  modified_base 30: mod_base = OTHER; note = 5-Methylcytosine
  modified_base 38: mod_base = OTHER; note = 5-Methylcytosine
  modified_base 41: mod_base = OTHER; note = 5-Methylcytosine
  modified_base 56: mod_base = OTHER; note = 5-methylcytosine
  modified_base 60: mod_base = OTHER; note = 5-methylcytosine
  modified_base 63: mod_base = OTHER; note = 5-Methylcytosine
aagttctgctatgtggcgcggtattatcccgtattgacgccgggcaagagcaactcggtcgccgcatacactatt

SEQ ID NO: 80 - DNA, 75 bp, Escherichia coli
  source 1..75: mol_type = genomic DNA; note = pUC19
ctcagaatgacttggttgagtactcaccagtcacagaaaggcatcttacggatggcatgacagtaagagaattat

SEQ ID NO: 81 - DNA, 75 bp, Escherichia coli
  source 1..75: mol_type = genomic DNA; note = pUC19
  modified_base 49: mod_base = OTHER; note = 5-Methylcytosine
ctcagaatgacttggttgagtactcaccagtcacagaaaagcatcttacggatggcatgacagtaagagaattat

SEQ ID NO: 82 - DNA, 75 bp, Escherichia coli
  source 1..75: mol_type = genomic DNA; note = pUC19
  modified_base 56: mod_base = OTHER; note = 5-Methylcytosine
ggcatgacagtaagagaattatgcagtgctgccataaccatgagtgataacactgcggccaacttacttctggca

SEQ ID NO: 83 - DNA, 75 bp, Escherichia coli
  source 1..75: mol_type = genomic DNA; note = pUC19
  modified_base 56: mod_base = OTHER; note = 5-Methylcytosine
ggcatgacagtaagagaattatgcagtgctgccataaccatgagtgataacactgcggccaacttacttctgaca

SEQ ID NO: 84 - DNA, 75 bp, Escherichia coli
  source 1..75: mol_type = genomic DNA; note = pUC19
  modified_base 2: mod_base = OTHER; note = 5-Methylcytosine
  modified_base 6: mod_base = OTHER; note = 5-Methylcytosine
  modified_base 14: mod_base = OTHER; note = 5-Methylcytosine
  modified_base 61: mod_base = OTHER; note = 5-methylcytosine
  modified_base 70: mod_base = OTHER; note = 5-methylcytosine
acgatcggaggacccgaggagctaaccgcttttttgcacaacatgggggatcatgtaactcgccttgatcgttgg

SEQ ID NO: 85 - DNA, 75 bp, Escherichia coli
  source 1..75: mol_type = genomic DNA; note = pUC19
  modified_base 2: mod_base = OTHER; note = 5-methylcytosine
  modified_base 6: mod_base = OTHER; note = 5-Methylcytosine
  modified_base 14: mod_base = OTHER; note = 5-methylcytosine
  modified_base 27: mod_base = OTHER; note = 5-methylcytosine
  modified_base 61: mod_base = OTHER; note = 5-methylcytosine
  modified_base 70: mod_base = OTHER; note = 5-methylcytosine
acgatcggaggacccaaggagctaaccgcttttttgcacaacatgggggatcatgtaactcgccttgatcgttgg

SEQ ID NO: 86 - DNA, 75 bp, Escherichia coli
  source 1..75: mol_type = genomic DNA; note = pUC19
  modified_base 5: mod_base = OTHER; note = 5-methylcytosine
  modified_base 65: mod_base = OTHER; note = 5-Methylcytosine
cttgcggatggcatgacagtaagagaattatgcagtgctgccataaccatgagtgataacactgcggccagctta

SEQ ID NO: 87 - DNA, 75 bp, Escherichia coli
  source 1..75: mol_type = genomic DNA; note = pUC19
  modified_base 5: mod_base = OTHER; note = 5-methylcytosine
  modified_base 65: mod_base = OTHER; note = 5-methylcytosine
cttacggatggcatgacagtaagagaattatgcagtgctgccataaccatgagtgataacactgcggccaactta

SEQ ID NO: 88 - DNA, 74 bp, Escherichia coli
  source 1..74: mol_type = genomic DNA; note = pUC19
  modified_base 10: mod_base = OTHER; note = 5-Methylcytosine
  modified_base 14: mod_base = OTHER; note = 5-Methylcytosine
  modified_base 22: mod_base = OTHER; note = 5-Methylcytosine
  modified_base 35: mod_base = OTHER; note = 5-Methylcytosine
  modified_base 69: mod_base = OTHER; note = 5-Methylcytosine
ttctgacaacgatcggaggacccgaggagctaaccgcttttttgcacaacatgggggatcatgtaactcgcctt

SEQ ID NO: 89 - DNA, 74 bp, Escherichia coli
  source 1..74: mol_type = genomic DNA; note = pUC19
  modified_base 10: mod_base = OTHER; note = 5-Methylcytosine
  modified_base 14: mod_base = OTHER; note = 5-Methylcytosine
  modified_base 22: mod_base = OTHER; note = 5-Methylcytosine
  modified_base 35: mod_base = OTHER; note = 5-Methylcytosine
  modified_base 69: mod_base = OTHER; note = 5-Methylcytosine
ttctgacaacgatcggaggacccaaggagctaaccgcttttttgcacaacatgggggatcatgtaactcgcctt

SEQ ID NO: 90 - DNA, 99 bp, Homo sapiens
  source 1..99: mol_type = genomic DNA
ggaagctggatggcccttgggggctgacccgcagggtgctggacttcgggatatagcagagagagcttggcatgacttattactccaggctgtagaatc

SEQ ID NO: 91 - DNA, 99 bp, Homo sapiens
  source 1..99: mol_type = genomic DNA
ggaagctggatggccctcagcggctgacccacagggtgccagattttgggacatagcagagagagcttggcacgtcttattactccaggctgttgaatc

SEQ ID NO: 92 - DNA, 99 bp, Homo sapiens
  source 1..99: mol_type = genomic DNA
ggaagctggatggccctaagcggctgacccacagggtgcaagaattaggganatagcagagagagttggcaacgacttattactccaggctgtagaatc